(19) United States
(12) Patent Application Publication
(10) Pub. No.: US 2023/0108125 A1
(43) Pub. Date: Apr. 6, 2023

(54) SMALL MOLECULE COMPOUNDS

(71) Applicant: Apertor Pharmaceuticals, Inc., South San Francisco, CA (US)

(72) Inventors: Edmund GRAZIANI, San Francisco, CA (US); Jay KEASLING, Berkeley, CA (US)

(21) Appl. No.: 17/849,600

(22) Filed: Jun. 25, 2022

Related U.S. Application Data
(60) Provisional application No. 63/215,189, filed on Jun. 25, 2021.

(51) Int. Cl. A61K 47/68; C12N 1/20; C12N 15/81; C12P 21/02

(52) U.S. Cl. CPC A61K 47/6803; C12N 1/205; C12N 15/81; C12P 21/02; C12R 2001/865

(57) ABSTRACT

Disclosed herein are pharmaceutical compositions comprising biosynthetic allosteric mTOR inhibitors that can have improved pharmacology and reduced toxicity. Also disclosed herein are methods of treating a condition or disease by administering biosynthetic allosteric mTOR inhibitors.

### CROSS-REFERENCE

This application claims the benefit of U.S. Provisional Application No. 63/215,189, filed on Jun. 25, 2021, which is incorporated by reference herein in its entirety.

### BACKGROUND

Neurodegenerative diseases ravages individuals, their families and caregivers, with astronomical costs to society (both in terms of affliction in the population as well as economically) that are only predicted to grow as the population ages. For example, Alzheimer's disease (AD), is the primary cause of dementia. The majority of cases of individuals with AD affects patients aged 65 and older. While a small number of treatments have shown benefits to patients, primarily in the alleviation of certain symptoms, there remains no treatment for the underlying neurodegeneration and no cure.

The emergence of novel RNA viruses can also result in high costs to society. In turn, this have led to an urgent need for the rapid development of effective vaccine formulations to treat such viral infections.

### SUMMARY

Provided herein are biosynthetic allosteric mTOR inhibitors. In some embodiments, the mTOR inhibitors have improved pharmacology and reduced toxicity. Provided herein certain embodiments are compounds useful as mTOR inhibitors.

Disclosed herein is a compound, or a pharmaceutically-acceptable salt or solvate thereof, having a structure represented by a structure of Formula (I):

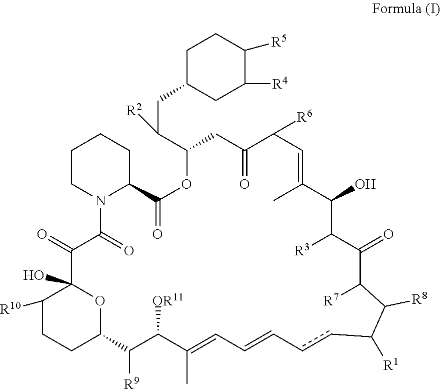

wherein, [symbol] is a single or a double bond; R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR₂₃R₂₄, —NR²¹C(═O)NR₂₃R₂₄, —NR₂₁S(═O)₂NR²³R₂₄, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R₂₄, —OC(═O)NR₂₃R₂₄, —NR₂₁C(═O)NR₂₃R₂₄, NR²¹S(═O)₂NR₂₃R₂₄, —NR₂₁C(═O)R²², —NR²¹C(═O)OR²¹, —C₁-C₈alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR₂₃R²⁴, —NR²¹C(═O)NR²³R²⁴, —NR²¹C(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R⁴ is —OH or —OCH₃; R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(R^{b})₂, —OP(═O)(R^{b})₂ or N₃; R⁶, R⁷, R⁸, R⁹ and R¹⁰ are each independently hydrogen, halogen, —CN, —OR²¹, —SR²¹, —S(═O)R²², —S(═O)₂R²², —NO₂, —NR₂₃R₂₄, —NR₂S(═O)₂R₂₂, —S(═O)₂NR₂₃R₂₄, —C(═O), R²², —OC(═O)R²², OC(═O)R²², —C₁-C₈-alkyl-C(═O)R²⁰, —C(═O)C(═O)R²², —C(═O)OR²¹, —C(═O)NR²¹, OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR₂₁C(═O)R₂₂, —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R²⁰ is independently halogen, —CN, —OR^{a}, —SR^{b}, —S(═O)₂R^{b}—NR^{c}R^{a}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{C}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a}; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d}; each R^{1a}, R^{1b}, R^{1c} and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl; each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R^{c} and R^{d} are taken together with the nitrogen atom to which they are attached to form a heterocycloalkyl which is optionally substituted; or a conjugate of the compound of Formula (I) to a biomolecule; wherein when R¹ is hydrogen, at least one of (i) R² is not CH₃, (ii) R³ is not OCH₃, or (iii) R⁴ is not —OCH₃; when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH—O(CH₂)₂OCH₂CH₃; —OC(═O)C(CH₂OH)₂CH₃; or OP(═O)(CH₃)₂; and when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃; (vi) R⁴ is not —OCH₃; or (vii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

In some embodiments, R¹ is hydrogen. In some embodiments, R¹ is CH₃. In some embodiments, R² is CH₃. In some embodiments, R³ is OCH₃. In some embodiments, R⁴ is OCH₃. In some embodiments, R⁴ is OH. In some embodiments, R⁵ is selected from the group consisting of:

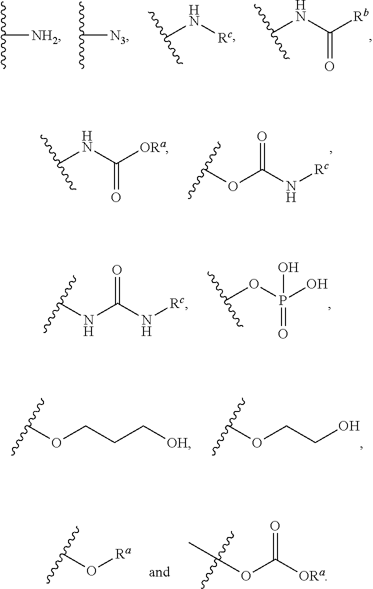

In some embodiments, R⁶ is CH₃. In some embodiments, R⁷ is CH₃. In some embodiments, R⁸ is hydrogen. In some embodiments, R⁹ is hydrogen. In some embodiments, R¹⁰ is CH₃. In some embodiments, R⁶ is CH₃. In some embodiments, at least one of R¹, R⁶, R⁷, R⁸, R⁹ or R¹⁰ is hydrogen, C₁-C₈ alkyl or C₂-C₈ alkenyl, each of which is unsubstituted or substituted with one or more R^{1e}, wherein R^{1e} is independently halogen, hydroxy, oxo, or phenyl.

In some embodiments, at least one of R¹, R⁶, R⁷, R⁸, R⁹ or R¹⁰ is hydrogen or a group selected from the group consisting of:

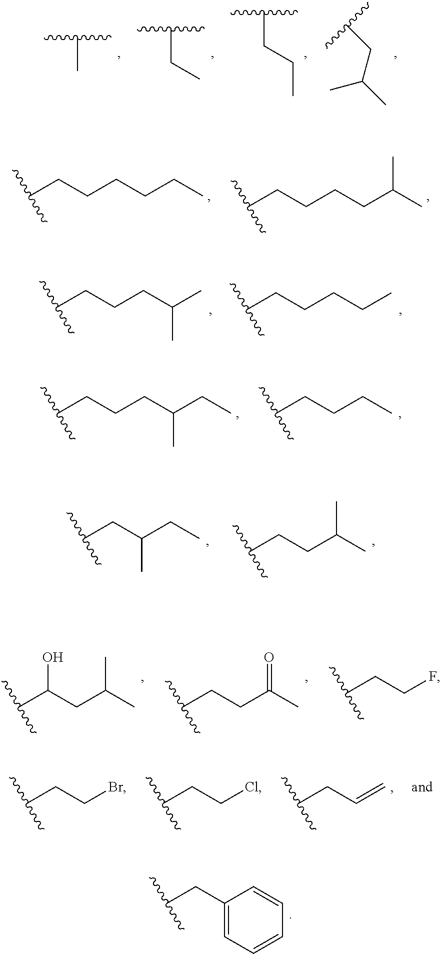

In some embodiments, the compound is selected from group from the consisting of:

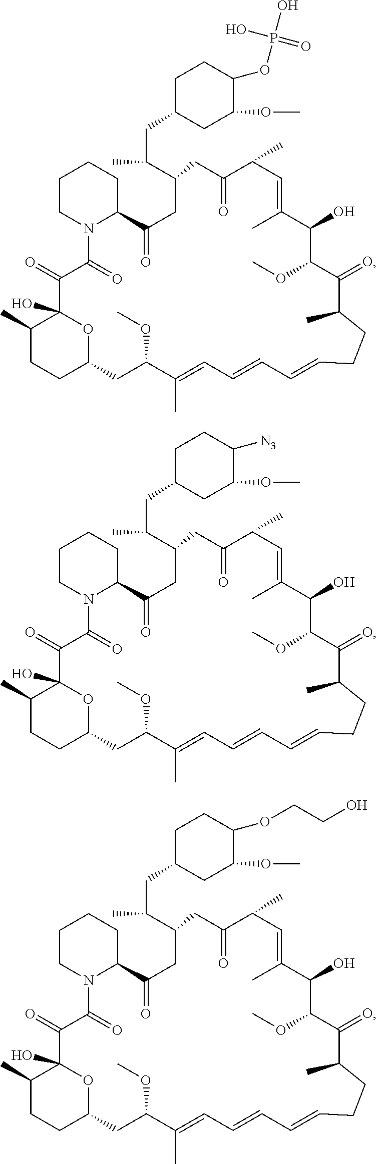

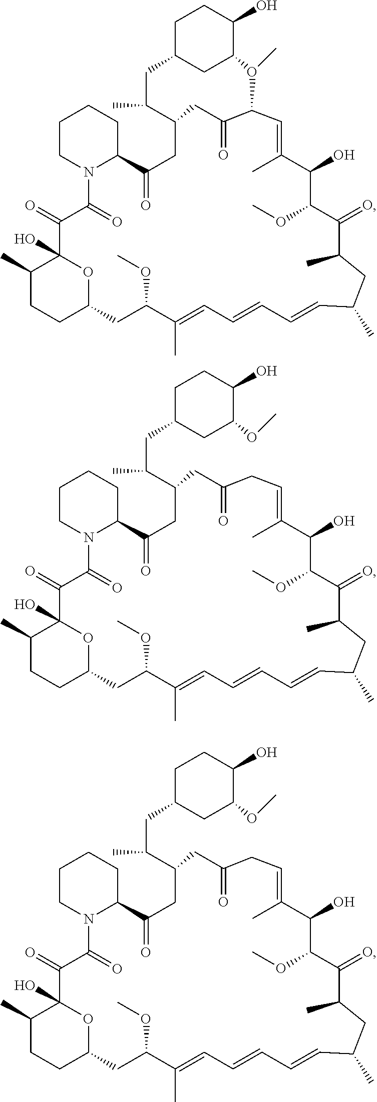

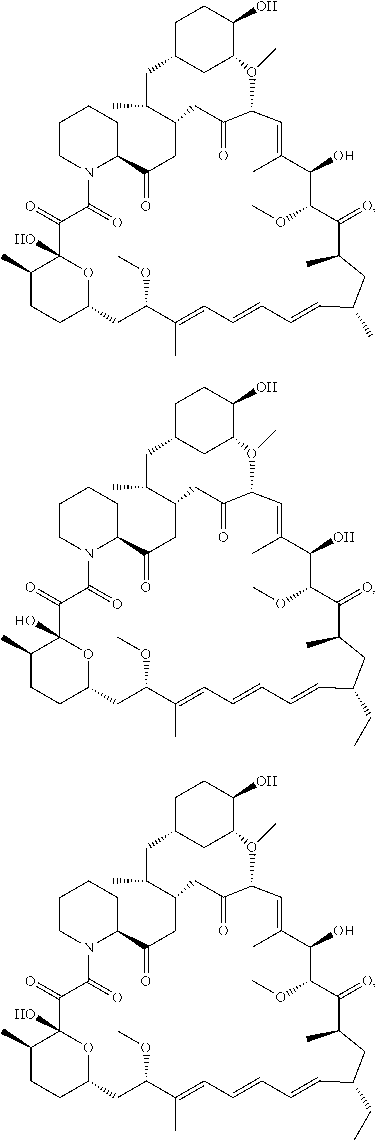

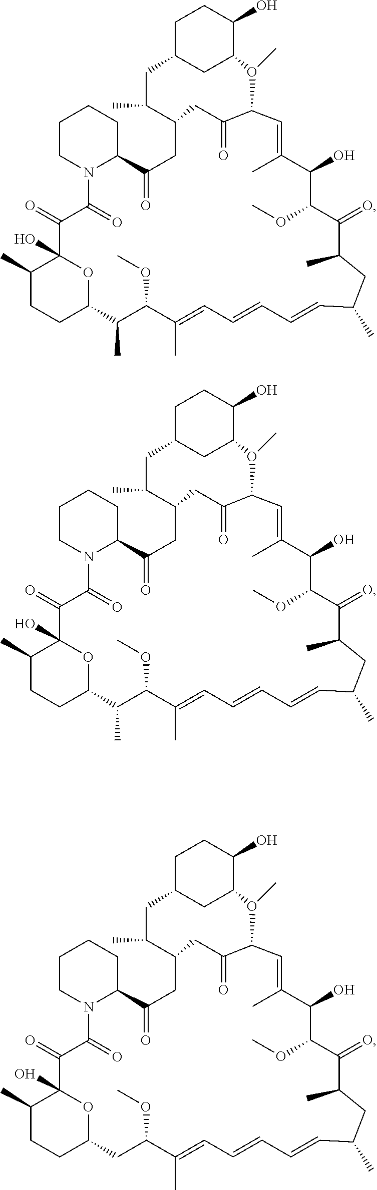

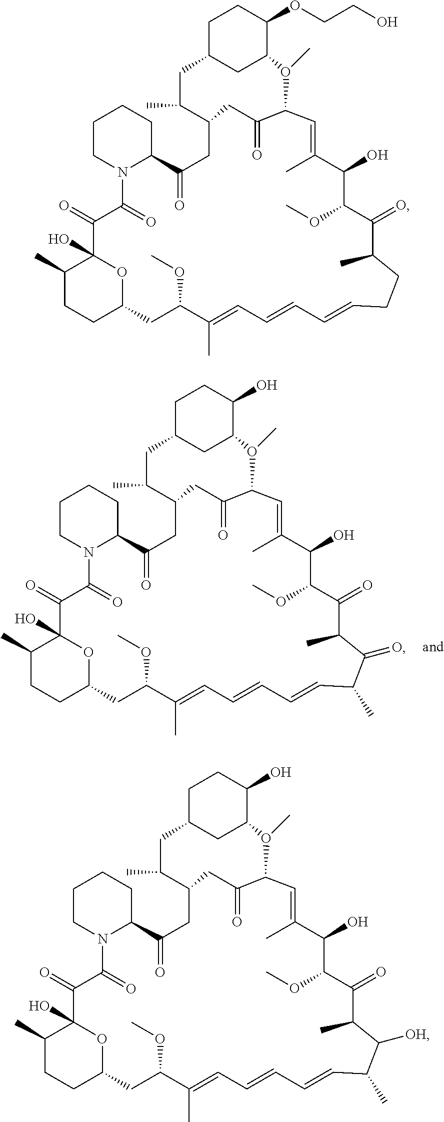

Disclosed herein is a compound, or a pharmaceutically-acceptable salt or solvate thereof, having a structure represented by a structure of Formula (I-A):

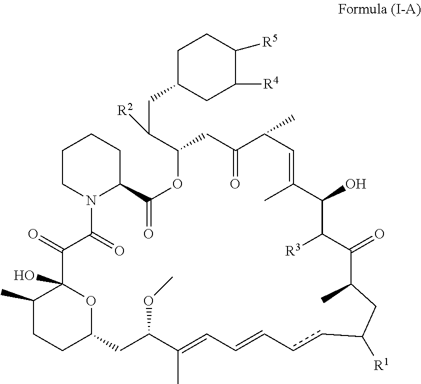

wherein, [symbol] is a single or a double bond; R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR₂₃R₂₄, —C(═O)R₂₂, —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR₂₃R₂₄, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, NR²¹S (═O)₂NR²³R²⁴, —NR²¹(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R⁴ is —OH, or —OCH₃; R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃; each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)R^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, Cₗ-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a}; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d}; each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, C(═O)_{R}^{b}, —OC(═O)R^{b}, —C(═O)R^{a}, —C(═O)SR^{b}, —OC(═O)R^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C═O)R^{b}, C₁-C₈ alkyl, —C₁-C₈alkenyl, C₁-C₈ haloalkyl, C₁-C₈ hydroxyalkyl, or phenyl; each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R^{c} and R^{d} are taken together with the nitrogen atom to which they are attached to form a heterocycloalkyl which is optionally substituted; or a conjugate of the compound of Formula (I-A) to a biomolecule; wherein when R¹ is hydrogen, at least one of (i) R² is not CH₃, (ii) R³ is not OCH₃, or (iii) R⁴ is not —OCH₃; when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and when R¹ is H or methyl, at least one of (i) R⁴ is not —OCH₃; or (ii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

In some embodiments, R¹ is hydrogen. In some embodiments, R¹ is CH₃. In some embodiments, R² is CH₃. In some embodiments, R³ is OCH₃. In some embodiments, R⁴ is OCH₃. In some embodiments, R⁴ is OH. In some embodiments, R⁵ is selected from the group consisting of:

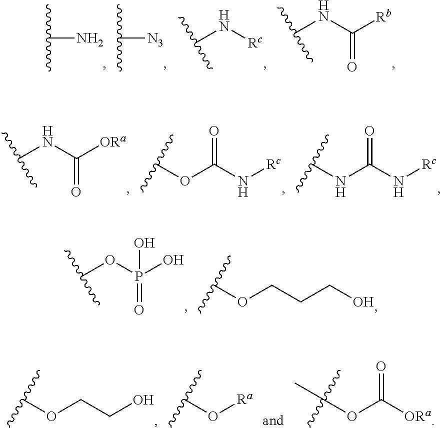

In some embodiments, R¹ is hydrogen, C₁-C₈ alkyl or C₂-C₈ alkenyl, each of which is unsubstituted or substituted with one or more R^{1e}, wherein R^{1c} is independently halogen, hydroxy, oxo, or phenyl.

In some embodiments, R¹ is hydrogen or a group selected from the group consisting of:

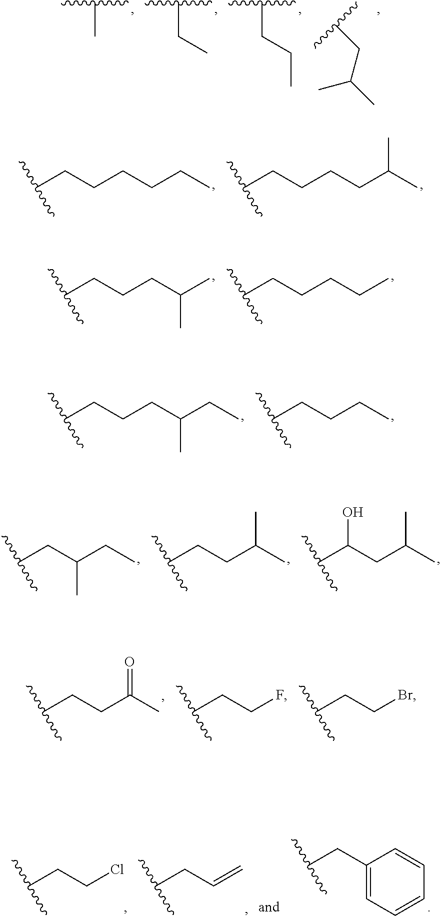

Disclosed herein is a compound, or a pharmaceutically-acceptable salt or solvate thereof, having a structure represented by a structure of Formula (I-B):

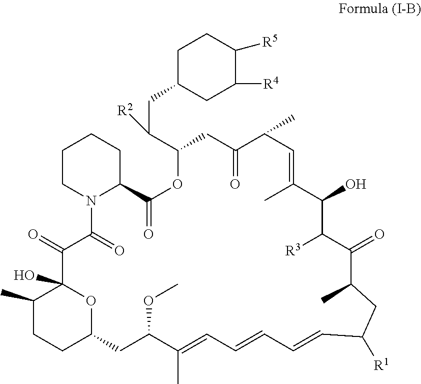

wherein, R¹ is hydrogen or CH₃; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R⁴ is —OH or —OCH₃; R⁵ is —OR^{a1}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃; each R²⁰ is independently halogen, —CN, —SR^{b}, —S(═O)₂R^{b}, —NR^{c}R^{d}, —S(═O)₂NR^{c}R^{d}, —C(═O)R^{b}, —OC(═O)R^{b}, —C(═O)R^{a}, —C(═O)SR^{b}, —OC(═O)R^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a}; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d}; each R^{1a}, R^{1b} R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl; each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R^{c} and R^{d} are taken together with the nitrogen atom to which they are attached to form a heterocycloalkyl which is optionally substituted; R^{a1} is C₁-C₈ alkyl, C₂-C₈ alkenyl, cycloalkyl, heterocycloalkyl, aryl, or heteroaryl; wherein the C₁-C₈ alkyl, cycloalkyl, heterocycloalkyl, aryl, and heteroaryl are each independently optionally substituted with one or more R²⁰; or a conjugate of the compound of Formula (I-B) to a biomolecule; wherein when Rⁱ is hydrogen, at least one of (i) R² is not CH₃, (ii) R³ is not OCH₃, or (iii) R⁴ is not —OCH₃; and when R¹ is CH₃, at least one of (i) R² is not CH₃, (ii) R³ is not hydrogen, —CH₃ or —OCH₃, (iii) R⁴ is not —OCH₃; or (iv) R⁵ not —O(CH₂)₂OH —O(CH₂)₂OCH₂CH₃; —OC(═O)C(CH₂OH)₂CH₃; or OP(═O)(CH₃)₂.

In some embodiments, R¹ is hydrogen. In some embodiments, R¹ is CH₃. In some embodiments, R² is CH₃. In some embodiments, R³ is OCH₃. In some embodiments, R⁴ is OCH₃. In some embodiments, R⁴ is OH.

In some embodiments, R⁵ is selected from the group consisting of:

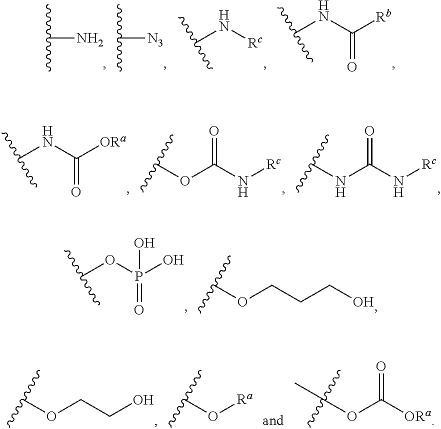

In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody.

Disclosed herein is a conjugate of a compound of formula (I), or a pharmaceutically-acceptable salt thereof, to a biomolecule

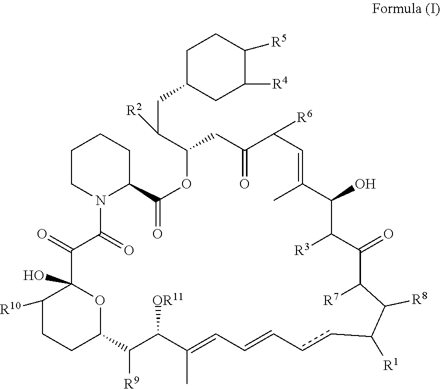

wherein, [symbol] is a single or a double bond; R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)_{R}²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R₂₂, —C(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R₂₄, —NR²¹(═O)R²², NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R³ is hydrogen, halogen, —OCH₃, —CN, —SR²¹, S(═O)R²², —S(═O)₂R²², —NO₂, —NR²³R²⁴, —NR²¹S(═O)₂R²², S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, NR²¹C(═O)NR²³R²⁴ —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R⁴ is —OH or —OCH₃; R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NRc—C(═O)OR^{a}, —NR^{c}—C(═O)—NR^{c}R^{d}, —OP(═O)(OR^{a})₂, —OP(═O)(R^{b})₂ or N₃; R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², S(═O)₂NR²³R²⁴, —C(═O)R²², —OC(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)R^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a}; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d}; each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl; each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R^{c} and R^{d} are taken together with the nitrogen atom to which they are attached to form a heterocycloalkyl which is optionally substituted; when R¹ is hydrogen, at least one of (i) R² is not CH₃, (ii) R³ is not OCH₃, or (iii) R⁴ is not —OCH₃; and when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH O(CH₂)₂OCH₂CH₃; —OC(═O)C(CH₂OH)₂CH₃; or OP(═O)(CH₃)₂.

In some embodiments, the compound of formula (I) is bound directly to the biomolecule. In some embodiments, the compound of formula (I) is bound to the biomolecule or through a linker (L). In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody. In some embodiments, the conjugate can comprise a plurality of compounds of formula (I) conjugated to the biomolecule. In some embodiments, the ratio of compound (I) to the biomolecule is about 100 to about 1. In some embodiments, the ratio of compound (I) to the biomolecule is 4. In some embodiments, the ratio of compound (I) to the biomolecule is 2.

Disclosed herein is a conjugate, or a pharmaceutically-acceptable salt or solvate thereof, having a structure represented by a structure of Formula (I-C):

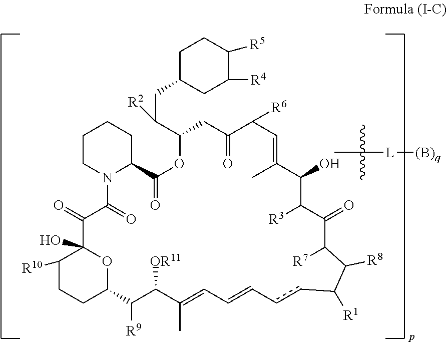

wherein, [symbol] is a single or a double bond; each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, NR²¹S(═O)₂NR²³R²⁴, —NR²¹(═O)R²², —NR²¹C(═O)OR²¹C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂R²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R⁴ is independently —OH or —OCH₃; each R⁵ is independently —OR^{a}, —OC(═O)R^{b}, —OC(═O)R^{a}, —OC(═O)—NR^{c}R^{d}, —NR^{c}R^{d}, —NR^{C}—C(═O)R^{b}, —NR^{c}—C(═O)R^{a}, —NR^{c}—C(═O)-NR^{c}R^{d}, —OP(═O)(OR^{a})₂, —OP(═O)(R^{b})₂ or N₃; R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R¹¹ is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R²⁰ is independently halogen, —CN, OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)R^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one lea; _{eac}h R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; or R²³ and R²⁴ are taken together with the nitrogen atom to which they are attached to form a heterocycloalkyl optionally substituted with one or more R^{1d}; each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)R^{a}, —C(═O)SR^{b}, —OC(═O)R^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NIVC(═O)R^{b}, C₁-C₈ alkyl, —C₂-C₈ alkenyl, C₁-C₈ haloalkyl, C₁-C₈ hydroxyalkyl, or phenyl; each R^{a}, R^{b}, R_{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R_{c} and R^{d} are taken together with the nitrogen atom to which they are attached to form a heterocycloalkyl which is optionally substituted; L is a linker; B is a biomolecule; p is 1-100; and q is 1-100; wherein when R¹ is hydrogen, at least one of (i) R² is not CH₃, (ii) R³ is not OCH₃, or (iii) R⁴ is not —OCH₃; and when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH O(CH₂)₂OCH₂CH₃; —OC(═O)C(CH₂OH)₂CH₃; or OP(═O)(CH₃)₂.

In some embodiments, the linker L is attached to R¹, R², R³, R⁴, R⁵, R⁶, R⁷, R⁸, R⁹, R¹⁰ or R¹¹. In some embodiments, linker L is attached to le, R², R³, R⁴, or R⁵. In some embodiments, the linker L is attached to R¹. In some embodiments, the linker L is attached to R⁵. In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody. In some embodiments, the ratio of p to q is 1:1 to 5:1. In some embodiments, p is 4 and q is 1. In some embodiments, p is 2 and q is 1.

Disclosed herein is an antibody-drug conjugate (ADC) having a structure represented by a structure of Formula (I-D):

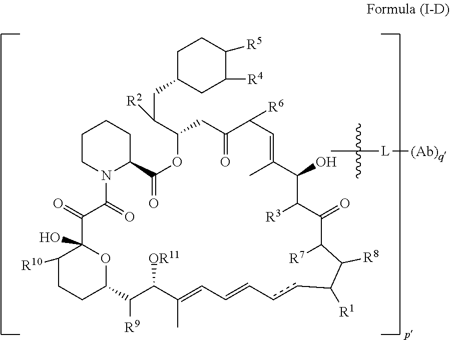

wherein, [symbol] is a single or a double bond; each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R² is independently hydrogen, halogen, —CN, —OR²¹, —SR²¹, —S(═O)R²², —S(═O)₂R₂₂, —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R³ is independently hydrogen, halogen, —OCH₃, —CN, SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R⁴ is independently —OH or —OCH₃; each R⁵ is independently —R^{a}, —OC(═O)R^{b}, —OC(═O)R^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR_{c}R^{d}, —OP(═O)(OR^{a})₂, —OP(═O)(R^{b})₂ or N₃; R⁶, R⁷, R⁸, R⁹ and R¹⁰ are each independently hydrogen, halogen, —CN, —OR²¹, —SR²¹, —S(═O)R²², —S(═O)₂R²², —NO₂, —NR₂₃R₂₄, —NR²¹S(═O)R²², —S(═O)₂NR²³R²⁴, —C(═O)R²², —OC(═O)R²², —C₁-C₇-alkyl-C(═O)R²⁰, —C(═O)C(═O)R²², —C(═O)OR²¹, —C(═O)NR²¹OR²¹, —OC(═O)OR²¹, —C(═O)OR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R²⁰ independently is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)R^{a}, —C(═O)SR^{b}, —OC(═O)R^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl, each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a}; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b}; or R²³ and R²⁴ are taken together with the nitrogen atom to which they are attached to form a heterocycloalkyl optionally substituted with one or more R^{1d}; each lea, R^{1b} R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)R^{1a}, —C(═O)SR^{b}, —OC(═O)R^{1a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl; each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted; or R^{c} and R^{d} are taken together with the nitrogen atom to which they are attached to form a heterocycloalkyl which is optionally substituted; L is a linker; Ab is an antibody or an antibody fragment; p′ is 1-100; and q′ is 1-100.

In some embodiments, the linker L is attached to R¹, R², R³, R⁴, R⁵, R⁶, R⁷, R⁸, R⁹, R¹⁰ or R¹¹. In some embodiments, the linker L is attached to R¹, R², R³, R⁴, or R⁵. In some embodiments, the linker L is attached to R¹. In some embodiments, the linker L is attached to R⁵. In some embodiments, the ratio of p′ to q′ is 1:1 to 5:1. In some embodiments, p′ is 4 and q′ is 1. In some embodiments, p′ is 2 and q′ is 1. In some embodiments, the linker L comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted. In some embodiments, the linker L comprises at least one group selected from the group consisting of —O—, —S—, —NH—, —NH—(CH₂)ₘ—NH; —NH—(CH₂)ₘ—O; —O—(CH₂)ₘ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂),(C═O)—, —(C═O)—(CH═CH)ₙ—(C═O), —(C═O)—(OCH₂CH₂O)ₙ—(C═O)—, —O(CH₂CH₂₀)ₙ—, —(C═O)—(CH₂CH₂O)ₙ—, and —(CH(CH₃)C(═O)O)ₙ—, wherein n is 1-20 and m is 1-20. In some embodiments, the linker L is or comprises at least one amino acid. In some embodiments, the linker L is or comprises two amino acids. In some embodiments, the linker L is or comprises three amino acids. In some embodiments, the linker L comprises at least one of valine, citrulline, valine-citrulline, glutamic acid-valine-citrulline, maleimidocaproylvaline-citrulline (mcValCit), lysine-valine-citrulline (LysValCit), N-acetyl-lysine-valine-citrulline (AcLysValCit), p-aminobenzoic acid (PABA), p-aminocarbamate (PABC), diaminoalkylene, 4-((2S)-2-((2S)-2-(6-(3-mercapto-2,5-dioxopyrrolidin-1-yl)hexanamido)-3-methylbutanamido)-5-ureidopentanamido)benzyl (2-aminoethyl)carbamate, or ((S)-5-acetamido-6-(((S)-1-(((S)-1-((4-((((2-aminoethyl)carbamoyl)oxy)methyl)phenyl)amino)-1-oxo-5-ureidopentan-2-yl)amino)-3-methyl-1-oxobutan-2-yl)amino)-6-oxohexyl)carbamic acid. In some embodiments, the linker L is a bond. In some embodiments, the compound of formula (I-C) or (I-D) is membrane-permeable.

Described herein is a pharmaceutical composition comprising a compound described herein, or a conjugate described herein, and at least one pharmaceutically-acceptable excipient. Described herein is an anti-aging pharmaceutical composition a compound described herein, or a conjugate described herein, and at least one pharmaceutically-acceptable excipient. In some embodiments, the pharmaceutical composition is in a unit dosage form. In some embodiments, the pharmaceutical composition described herein can be in a form suitable for oral, parenteral, rectal, ocular, intravenous or otic administration, or administration by inhalation.

Described herein is a method of treating a condition or disease in a subject in need thereof, comprising administering a pharmaceutical composition described herein. Described herein is method of treating a condition or disease in a subject in need thereof, comprising administering a compound described herein or a pharmaceutical composition described herein, thereby treating the condition or disease in the subject. Described herein is use of a compound described herein or a pharmaceutical composition described herein for the treatment of a condition or disease in a subject in need thereof, the use comprising administering to the subject the compound or the pharmaceutical composition, thereby treating the condition or disease in the subject. Described herein is use of a compound described herein or a pharmaceutical composition described herein for the manufacture of a medicament for the treatment of a condition or disease in a subject in need thereof, the use comprising administering to the subject an effective amount of the compound or the pharmaceutical composition, thereby treating the condition or disease in the subject. In some embodiments, administering the compound or the pharmaceutical composition results in inhibiting mTORC₁ and/or mTORC₂. In some embodiments, administering the pharmaceutical composition further results in promoting immune cell differentiation In some embodiments, administering the pharmaceutical composition results in suppression of proliferation of effector T-cells. In some embodiments, administering the pharmaceutical composition results in differentiation of memory T-cells. In some embodiments, administering the pharmaceutical composition results in differentiation of regulatory T-cells. In some embodiments, administering the pharmaceutical composition results in cellular senescence. In some embodiments, the condition or disease is a neurodegenerative disease. In some embodiments, the neurodegenerative disease is Alzheimer Disease, Parkinson Disease, Parkinson-like Disease, Huntington Disease, Lou Gehrig Disease, Multiple Sclerosis, autoimmune disorders, Pick Disease, diffuse Lewy body Disease, progressive supranuclear palsy (Steel-Richardson syndrome), multisystem degeneration (Shy-Drager syndrome), motor neuron diseases, amyotrophic lateral sclerosis, degenerative ataxias, cortical basal degeneration, ALS-Parkinson-Dementia complex of Guam, subacute sclerosing panencephalitis, synucleinopathies, primary progressive aphasia, striatonigral degeneration, Machado-Joseph disease/spinocerebellar ataxia type 3 and olivopontocerebellar degenerations, Gilles De La Tourette Disease, bulbar and pseudobulbar palsy, spinal and spinobulbar muscular atrophy (Kennedy Disease), primary lateral sclerosis, familial spastic paraplegia, Werdnig-Hoffmann Disease, Kugelberg-Welander Disease, Tay-Sach Disease, Sandhoff Disease, familial spastic disease, Wohlfart-Kugelberg-Welander Disease, spastic paraparesis, progressive multifocal leukoencephalopathy, prion diseases, including Creutzfeldt-Jakob Disease, Gerstmann-Straussler-Scheinker Disease, Kuru, and fatal familial insomnia. In some embodiments, the neurodegenerative disease is Alzheimer Disease. In some embodiments, the condition or disease is a viral infection. In some embodiments, the viral infection is caused by a coronavirus. In some embodiments, the coronavirus is Alphacoronavirus, Betacoronavirus, a Gammacoronavirus, Deltacoronavirus, 229E coronavirus, NL63 coronavirus, OC₄₃ coronavirus, HKU1 coronavirus, middle east respiratory syndrome related coronavirus (MERS-CoV), severe acute respiratory syndrome coronavirus (SARS-CoV), severe acute respiratory syndrome coronavirus 2 (SARS-CoV-2), a mutated form of any of these, or any combination thereof.

In some embodiments, administering the pharmaceutical composition further comprises co-administration of a vaccine. In some embodiments, co-administration results in improved effectiveness of the vaccine. In some embodiments, administering the pharmaceutical composition is effective to at least partially reduce a viral load of a coronavirus. In some embodiments, the subject has or was previously diagnosed with a general symptom of a coronavirus. In some embodiments, the general symptom comprises a fever, a cough, shortness of breath, breathing difficulties, or any combination thereof.

Described herein is a kit comprising a compound described herein, a conjugate described herein, or a pharmaceutical composition described herein. In some embodiments, the kits can further comprise instructions for using the pharmaceutical composition. In some embodiments, the kits can further comprise a coronavirus vaccine.

Described herein is a process for producing a biomolecule-drug-conjugate, comprising: (a) providing a compound of formula (I), (I′), (I-A) or (I-B) as described herein; linking a linker L to the compound of (a) to obtain a conjugate; and (b) linking the conjugate of step (b) to a biomolecule so as to obtain a biomolecule-drug-conjugate. Described herein is a process for producing a biomolecule-drug-conjugate, comprising: (a) providing a compound of formula (I), (I′), (I-A) or (I-B) as described herein linking the compound (a) to a biomolecule so as to obtain a biomolecule-drug-conjugate. In some embodiments, the process can further comprise purifying the biomolecule-drug-conjugate. In some embodiments, the biomolecule is an antibody. In some embodiments, the conjugating is site specific on one or more engineered cysteine and/or glutamine residues on the antibody.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

While various embodiments of the invention have been shown and described herein, it may be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### TERMINOLOGY

Unless specifically stated or obvious from context, as used herein, the term “about” in reference to a number or range of numbers is understood to mean the stated number and numbers ±10% thereof, or 10% below the lower listed limit and 10% above the higher listed limit for the values listed for a range.

The singular forms “a”, “an”, and “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“Effective amount,” “sufficient amount,” and “amount sufficient for” may be used interchangeably and refer to an amount of a substance that is sufficient to achieve an intended purpose or objective.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

A “therapeutically effective amount” when used in connection with a pharmaceutical composition described herein is an amount of one or more pharmaceutically active agent(s) sufficient to produce a therapeutic result in a subject in need thereof. An “amount” of one or more components in the pharmaceutical composition refers to an amount per unit dose.

The term “pharmaceutically-acceptable” denotes an attribute of a material which is useful in preparing a pharmaceutical composition that is generally safe, non—toxic, and neither biologically nor otherwise undesirable and is acceptable for veterinary as well as human pharmaceutical use. “Pharmaceutically-acceptable” can refer a material, such as a carrier or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

The term “derivative” as used herein indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In various instances, “may” refers to optional alternatives to be used in the alternative or in addition to other specified components.

“Alkyl” refers to a straight or branched hydrocarbon chain radical consisting solely of carbon and hydrogen atoms, which may optionally be unsaturated with one or more double or triple bonds, and preferably having from one to fifteen carbon atoms (i.e., C₁-C₁₅ alkyl). In certain embodiments, an alkyl comprises one to six carbon atoms (i.e., C₁-C₆ alkyl). In other embodiments, an alkyl comprises one to three carbon atoms (i.e., C₁-C₃ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless otherwise specified, the term “alkyl” and its equivalents encompass linear, branched, and/or cyclic alkyl groups. In some instances, an “alkyl” comprises both cyclic and acyclic (linear and/or branched) alkyl components. When an alkyl group is described as “linear,” the referenced alkyl group is not substituted with additional alkyl groups and is unbranched. When an alkyl group is described as “saturated,” the referenced alkyl group does not contain any double or triple carbon-carbon bonds (e.g. alkene or alkyne).

“Alkylene” or “alkylene chain” refers to a divalent alkyl group, which may optionally be substituted as defined herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described herein. “Alkenylene” or “alkenylene chain” refers to a divalent alkenyl group, which may optionally be substituted as described herein.

“Alkynyl” refers to a straight or branched hydrocarbon chain radical group consisting solely of carbon and hydrogen atoms, containing at least one carbon-carbon triple bond, and having from two to twelve carbon atoms. In certain embodiments, an alkynyl comprises two to eight carbon atoms. In other embodiments, an alkynyl comprises two to four carbon atoms. The alkynyl is optionally substituted as described herein. “Alkynylene” or “alkynylene chain” refers to a divalent alkynyl group, which may optionally be substituted as described herein.

“Heteroalkyl” refers to an alkyl group wherein one or more of the carbons of the alkyl group is replaced with a heteroatom. Exemplary heteroatoms include N, O, Si, P, B, and S atoms, preferably N, O and S. Note that valency of heteroatoms may not be identical to that of a carbon atom, so, for example, a methylene (CH₂) of an alkyl may be replaced with an NH group, S group, O group, or the like in a heteroalkyl.

“Heteroalkylene” refers to a divalent heteroalkyl group defined above which links one part of the molecule to another part of the molecule. Unless stated specifically otherwise, a heteroalkylene is optionally substituted as defined herein.

“Aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π—electron system in accordance with the Hiickel theory. The ring system from which aryl groups are derived include, but are not limited to, groups such as benzene, fluorene, indane, indene, tetralin and naphthalene.

“Arylene” group as used herein refers to a divalent aryl group, wherein aryl is as defined herein. The arylene group may optionally be substituted as defined herein.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “Cₓ-C_{y}alkyl” refers to saturated or unsaturated hydrocarbon groups, including straight-chain alkyl and branched-chain alkyl groups that contain from x to y carbons in the chain. The terms “C_{x-y}alkenyl” and “C_{x-y}alkynyl” refer to unsaturated aliphatic groups analogous in length and possible substitution to the alkyls described above, but that contain at least one double or triple bond respectively.

“Cycloalkyl” refers to a saturated ring in which each atom of the ring is carbon. Cycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Cycloalkylene” refers to a divalent cycloalkyl group , which may optionally be substituted as defined herein.

“Halo” or, alternatively, “halogen” or “halide,” means fluoro, chloro, bromo or iodo. In some embodiments, halo is fluoro, kechloro, or bromo.

“Haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

“Heterocycloalkyl” refers to a saturated or unsaturated (e.g., non-aromatic) ring with carbon atoms and at least one heteroatom (e.g., a cycloalkyl wherein one or more of the carbon groups is substituted with a heteroatom). Exemplary heteroatoms include N, O, Si, P, B, and S atoms. Heterocycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocycloalkylene” refers to a divalent heterocycloalkyl which may optionally be substituted as defined herein. In some embodiments, the heterocycloalkylene is monocyclic. In some embodiments, the heterocycloalkylene is bicyclic.

“Heteroaryl” refers to an aromatic ring comprising carbon atoms and one or more heteroatoms. Exemplary heteroatoms include N, 0, Si, P, B, and S atoms. As used herein, the heteroaryl ring may be selected from monocyclic or bicyclic and fused or bridged ring systems rings wherein at least one of the rings in the ring system is aromatic, i.e., it contains a cyclic, delocalized (4n+2) 7c—electron system in accordance with the Htickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

“Heteroarylene” refers to a divalent heteroaryl which may optionally be substituted as defined herein. In some embodiments, the heteroaryl is monocyclic. In some embodiments, the heteroaryl is bicyclic.

The term “substituted” refers to moieties having substituents replacing a hydrogen on one or more carbons or heteroatoms of the structure. It may be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds.

For purposes of this disclosure, the heteroatoms such as nitrogen may have hydrogen substituents and/or any permissible substituents of organic compounds described herein which satisfy the valences of the heteroatoms. In embodiments where it is unspecified whether a group is substituted or unsubstituted, it is intended that the group is unsubstituted.

Substituents may include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b′}—OC(O)-R^{a′}, —R^{b′}—OC(O)—OR^{a′}, —R^{b′}—OC(O)—N(R^{a′})₂, —R^{b′}—N(R^{a})₂, —R^{b′}—C(O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{a′}, —R^{b′}—N(R^{a′})C(O)R^{a′}, —R^{b′}—N(R^{a′})S(O)ₜR^{a′} (where t is 1 or 2), —R^{b′}—S(O)ₜR^{a′} (where t is 1 or 2), —R^{b′}—S(O)ₜOR^{a′} (where t is 1 or 2), and —R^{b′}—S(O)ₜN(R^{a′})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b′}—OR^{a′}, —R^{b′}—OC(O)—R^{a′}, —R^{b′}—OC(O)—N(R^{a′})₂, —R^{b′}-N(R^{a′})₂, —R^{b′}—C(O)R^{a′}, —R^{b′}—C(O)N(R^{a′})₂, —R^{b′}-—C(O)N(R^{a′})₂, —R^{b′}—O—R^{c′}—C(O)N(R^{a′})₂, —R^{b′}-N(R^{a′})C(O)OR^{a′}, —R^{b′}—N(R^{a′})C(O)R^{a′}, —R^{b′}—N(R^{a′})S(O)ₜR^{a′} (where t is 1 or 2), —R^{b′}—S(O)ₜR^{a′} (where t is 1 or 2), —R^{b′}—S(O)ₜOR^{a′} (where t is 1 or 2) and —R^{b′}—S(O)ₜN(R^{a′})₂ (where t is 1 or 2); wherein each R^{a′} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a′}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (=N—OH), hydrazine (═NNH₂), —R^{b′}—OR^{a′}, —R^{b′}—OC(O)—OR^{a′}, —R^{b′}—OC(O)—, —R^{b′}—OC(O)—N(R^{a′)}₂, —R^{b′})₂, —R^{b′}—C (O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{a′}, —R^{b′}—N( R^{a′})C(O)R^{a′}, —R^{b}—N(R^{a′})S(O)ₜR^{a′} (where t is 1 or 2), —R^{b′}—S(O)ₜR^{a′}(where t is 1 or 2), —R^{b′}—S(O)ₜOR^{a′} (where t is 1 or 2) and —R^{b′}—S(O)ₜN(R^{a′})₂ (where t is 1 or 2); and wherein each R^{b′} is independently selected from a direct bond or a straight or branched alkylene, alkenylene, or alkynylene chain, and each R^{c′} is a straight or branched alkylene, alkenylene or alkynylene chain.

Compounds of the present disclosure also include crystalline and amorphous forms of those compounds, pharmaceutically-acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

### MTOR INHIBITORS

mTOR Inhibitors—Alzheimer's Disease

In Alzheimer's Disease (AD), the formation of β-amyloid plaques in the brain (amyloid cascade) has been hypothesized as the leading cause of the disease. Therapeutics aimed to prevent, slow neuronal loss or reversing the formation of β-amyloid plaques may treat AD. However, while amyloid deposits have been shown to form decades prior to the manifestation of clinical symptoms, cognitive deficits and synapse loss more closely correlate with abnormal function of the protein, tau (also called microtubule associated protein, MAPT). Thus, approaches in treating AD can also investigate the role of microglia—the innate immune cells in the central nervous system (CNS)—in the pathogenesis of AD. Microglia are known to play an important role in the forgetting of remote memories via synapse elimination; furthermore, there may be evidence that dysregulation and uncontrolled proliferation of microglia can be harmful to neurons. For example, mutations in the microglial surface receptor, TREM2, can affect microglial function in models of AD. This suggests a dimension of AD that is similar to autoimmune disorders, and while there remain contradictory aspects to this hypothesis, it is not unreasonable to consider AD as a disease of the aging immune system.

The biology of aging and mechanisms of cellular senescence are currently poorly understood. The role of senescence in immune cell function is complicated, as there are orders-of-magnitude of increased complexity when attempting to untangle the role of T-cell and B-cell proliferation and differentiation in normal immune function. However, there may be evidence that CD4+ CD25+ forkhead box P3 (Foxp3)+regulatory T lymphocytes (Tregs) could be important neuroprotective modulators of microglial function, with depletion of Tregs negatively affecting outcome in models of neuronal injury. The serine/threonine kinase target of rapamycin (mTOR) may be a key metabolic switch that controls the proliferation and differentiation of naïve T cell lineages into Tregs.

Mechanistic target of rapamycin (mTOR) is a highly conserved member of the phosphatidylinositol 3-kinase family that exists in two protein complexes, mTORC₁ and mTORC₂. Inhibition of mTORC₁ signaling by the microbial natural product, rapamycin and its cytosolic chaperone immunophilin, FK-506 binding protein 12 (FKBP12), may suppresses proliferation of effector T-cells and may induce differentiation of memory and regulatory T-cells (T_{reg}) in humans. Regulatory T-cells are a subset of anti-inflammatory T cells that dampen an immune response through secretion of immunomodulatory cytokines (e.g., IL-10, TGFβ), checkpoint inhibitor interaction (e.g., CTLA-4, LAG-3) and competition for essential metabolites and cytokines. Without wishing to be bound to any theory, for a therapeutic to maximize efficacy within an autoimmune disease setting, it would need to reduce conventional T cell proliferation and activation whilst preserving or enhancing the generation or phenotype of T_{reg} cells. Due to the allosteric inhibition of mTORC₁ that may lead to both a milder immunosuppression (proliferation of T cells) and sustained differentiation and proliferation of Tregs, antagonists of mTORC₁ may provide a venue for treating neurodegenerative diseases (e.g., Alzheimer's Disease).

mTOR Inhibitors—Potential Vaccine Adjuvants

The emergence of novel RNA viruses as vectors of life-threatening pandemics underlines the urgency for the rapid development of vaccines against these pathogens. Drugs that enhance the ability of the adaptive immune system to respond to viral infections can be useful as adjuvants co-administered with vaccines, thus increasing their effectiveness, particularly in older patients, cancer patients and patients with underlying medical disorders. Productive immunization to viral antigens may hinge on responses from both arms of the adaptive immune system—T and B lymphocytes—and attempts to improve vaccine responses or improve host responses against a viral infection in elderly patients may have to rely on strengthening T-cell immunity.

mTORC1 inhibition may promote immune cell differentiation that can lead to more robust and effective responses to vaccines. The immune-enhancing effects of rapamycin have been shown for CD8+ T cells. Inhibitors of mTORC_{1/}C₂ may significantly reduce infections in the elderly when co-administered with a seasonal influenza vaccine. The combination of an orthotopic inhibitor at the ATP-binding site of mTORC₁ (dactolisib, a pan-phosphoinositide 3-kinase (PI3K)/mTOR inhibitor) and an allosteric mTORC₁ inhibitor (everolimus) administered for 6 weeks prior to immunization in a Phase II clinical study showed significantly increased antibody titers to influenza virus vaccine strains, and reduced the aforementioned incidence of respiratory infections for a year. A Phase 3 clinical study focusing on determining if using dactolisib alone prevents illness associated with respiratory tract infections in people >65 years of age. However, the study was discontinued and the rationale used for testing dactolisib alone (without everolimus) in this study is unclear, which left open the possibility that the positive results observed in the Phase II study were attributable, wholly or in part, to everolimus. This observation outlined a path to discover a single agent that can selectively modulate the role of mTOR in both innate and adaptive responses in T-cells that would have significant therapeutic potential for protecting the population from viral respiratory infections. Interestingly, a SARS-CoV-2-human protein-protein interaction map revealed direct viral-human interactions with proteins regulated by the mTORC₁ pathway, such as LARP1, and FKBP7, which interact with the viral N and Orf8 proteins, respectively, suggesting the added possibility of direct mTORC₁ inhibition affecting viral replication.

The rapidity with which SARs-CoV2 vaccines are being developed is quite impressive, with at multiple clinical trials already up and concurrently running. This, however, makes even more urgent the need to develop therapeutics to ensure that these vaccines are effective in the most vulnerable populations. Therefore, there is a need to design and screen for compounds that are partial allosteric antagonists of mTORC₁ signaling such that downstream events that control proliferation are ablated, while effects on differentiation to regulatory and memory phenotypes are preserved. In this way, a single agent can combine sufficient but partial mTORC₁ inhibition to enhance innate antiviral immunity while at the same time possessing an optimized magnitude and duration of inhibition that preserves and potentially improves the enhancement of adaptive immunity.

### Rapamycin and Derivatives

Previous medicinal chemistry work on rapamycin was focused on designing compounds for use in treating cancer, and thus the observation that modifications to the macrocyclic ring of rapamycin led to reduced antiproliferative activity of tumor cells led to the prioritization of compounds that retained full binding to mTORC₁. Hence, the four rapamycin analogs that are currently FDA approved are essentially identical, as all are modified with solubilizing groups at the C-42 hydroxyl position.

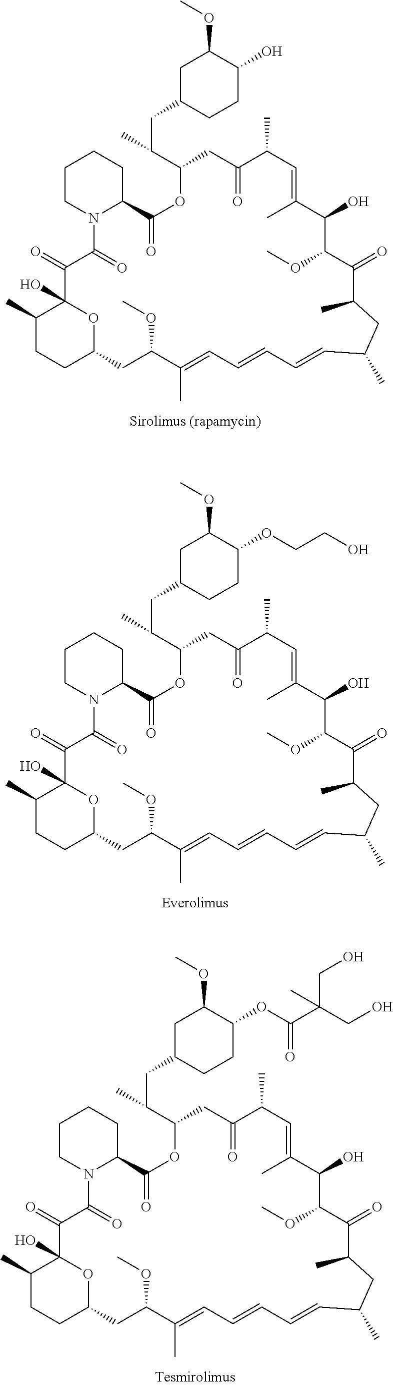

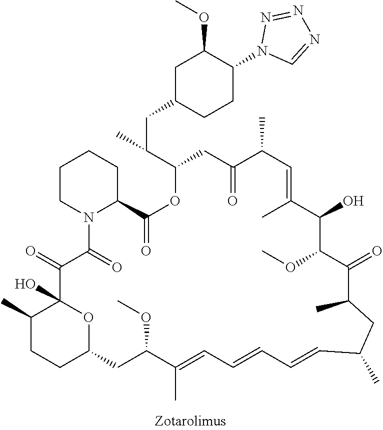

In some embodiments, the compounds described herein consider the structural features that can be important for binding to FKBP12 and combined with additional variables to alter the terminal half-life in vivo. As partitioning into the red blood cells is thought to be driven by binding to immunophilins such as FKBPs, this may spare the compounds described herein from first pass metabolism and lead to the long terminal half-life (24-48 hours) observed in humans with rapamycin.

In some embodiments, the compounds described herein can be synthesized using synthetic biology techniques to engineer the rapamycin polyketide synthase in Streptomyces rapamycinicus for producing specific alterations to the structure of products generated through exchange acyltransferases that specify substituents at the a-carbons of the Claisen-type products of PKS assembly and accompanying cytochrome P-450s.

### COMPOUNDS

The present disclosure provides compounds and salts, and formulations thereof, for use in treating various diseases. In some embodiments, disclosed herein, is a compound, or a pharmaceutically-acceptable salt or solvate thereof, having a structure represented by a structure of Formula (I):

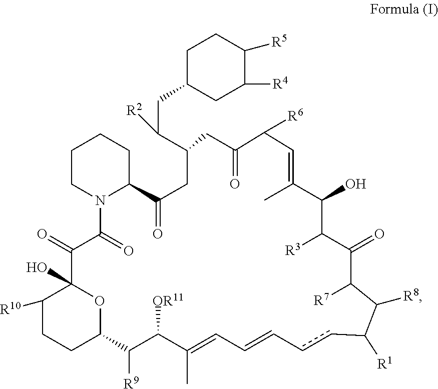

wherein, [symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC (═O)NR²³R²⁴, —NR²¹C(═O)NR²³R²⁴, —NR²¹S(═O)₂NR²³R²⁴, NR²¹C(═O)R²², —NR²¹C(═O)OR²¹, NR²¹(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²², —C(═O)C(═O)R²², —C(═O)OR²¹, —C(═O)NR²¹OR²¹, —OC(═O)OR²¹, —C(═O)NR²³R²⁴, —NR²¹C(═O)_{NR}²³R²⁴, —NR²¹S(═O)₂NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of formula (I) to a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH, —O(CH₂)₂OCH₂CH₃; —OC(═O)C(CH₂OH)₂CH₃; or OP(═O)(CH₃)₂; and
(c) when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃, (vi) R⁴ is not —OCH₃; or (vii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

In some embodiments, disclosed herein, is a compound, or a pharmaceutically-acceptable salt or solvate thereof, having a structure represented by a structure of Formula (I′):

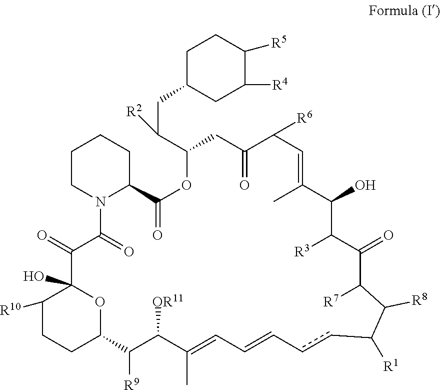

wherein,
[symbol] is a single or a double bond;
R¹ is hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R² is hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R³ is hydrogen, halogen, —OCH₃, —CN, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R¹¹ is hydrogen, alkyl, aryl or heteroaryl, wherein the alkyl, aryl and heteroaryl are each independently optionally substituted;
each R^{a}, R^{b}, R^{c}, and R^{d} is independently hydrogen, alkyl, aryl or heteroaryl, wherein the alkyl, aryl and heteroaryl are each independently optionally substituted;
or R^{c} and R^{d} are taken together with the nitrogen atom to which they are attached to form a heterocycloalkyl which is optionally substituted;
or a conjugate of the compound of formula (I) to a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
(c) when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃, (vi) R⁴ is not —OCH₃; or
(vii) R⁵ is not —OH or —OC(═O)R^{b}, wherein R^{b} is alkyl, aryl or heteroaryl, wherein the alkyl, aryl and heteroaryl are each independently optionally substituted.

In some embodiments of Formula (I) or (I′), R¹ is hydrogen. In some embodiments of Formula (I), R¹ is CH₃. In some embodiments of Formula (I) or (I′), R² is CH₃. In some embodiments of Formula (I) or (I′), R³ is OCH₃. In some embodiments of Formula (I) or (I′), R⁴ is OCH₃. In some embodiments of Formula (I) or (I′), R⁴ is OH. In some embodiments of Formula (I) or (I′), R⁵ is selected from the group consisting of:

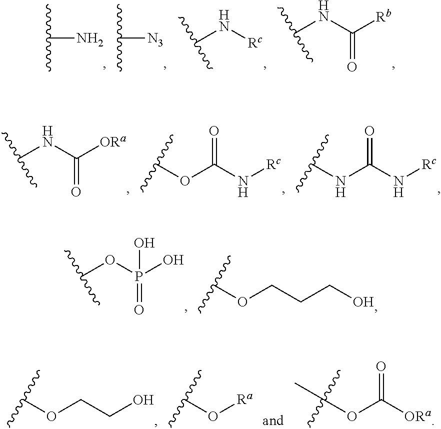

In some embodiments of Formula (I) or (I′), R⁶ is CH₃. In some embodiments of Formula (I), R⁷ is CH₃. In some embodiments of Formula (I) or (I′), R⁸ is hydrogen. In some embodiments of Formula (I) or (I′), R⁹ is hydrogen. In some embodiments of Formula (I) or (I′), R¹⁰ is CH₃. In some embodiments of Formula (I) or (I′), R¹¹ is CH₃. In some embodiments of Formula (I) or (I′), at least one of R¹, R⁶, R⁷, R⁸, R⁹ or R¹⁰ is hydrogen, C₁-C₈ alkyl or C₂-C₈ alkenyl, each of which is unsubstituted or substituted with one or more R^{1e}, and R^{1e} is independently halogen, hydroxy, oxo, or phenyl.

In some embodiments of Formula (I) or (I′), at least one of R¹, R⁶, R⁷, R⁸, R⁹ or R¹⁰ is hydrogen or a group selected from the group consisting of:

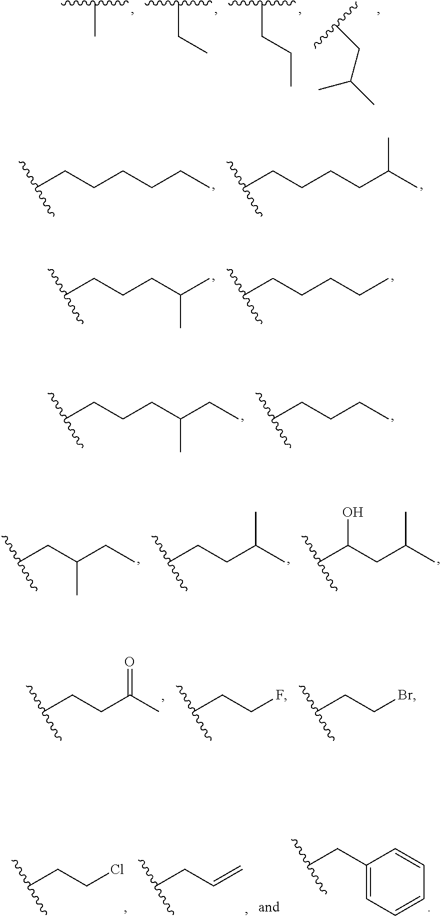

In some embodiments, disclosed herein, is a compound, or a pharmaceutically-acceptable salt or solvate thereof, having a structure represented by a structure of Formula (I-A):

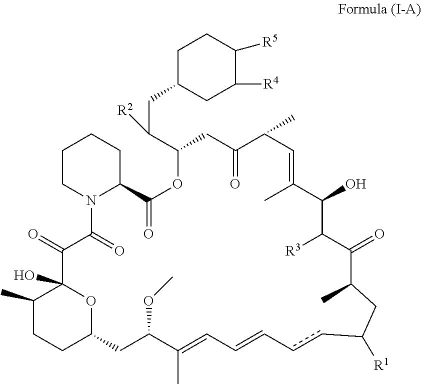

wherein,
[symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹(═O)NR²³R²⁴, —NR²¹S(═O)₂NR²³R²⁴, —NR²¹C(═O)R²², —NR²¹C(═O)OR²¹, NR²¹(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, -—OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH, or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a1}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of formula (I-A) with a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
(c) when R¹ is H or methyl, at least one of (i) R⁴ is not —OCH₃; or (ii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

In some embodiments of Formula (I-A), R¹ is hydrogen. In some embodiments of Formula (I-A), R¹ is CH₃. In some embodiments of Formula (I-A), R² is CH₃. In some embodiments of Formula (I-A), R³ is OCH₃. In some embodiments of Formula (I-A), R⁴ is OCH₃. In some embodiments of Formula (I-A), R⁴ is OH. In some embodiments of Formula (I-A), R⁵ is selected from the group consisting of:

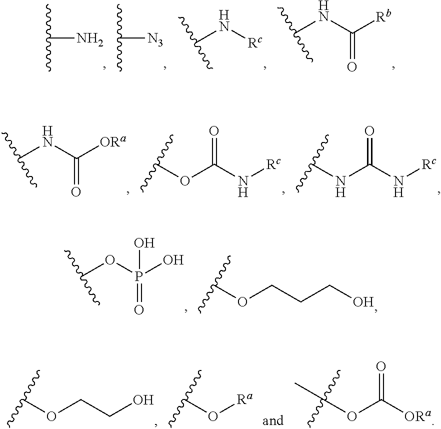

In some embodiments of Formula (I-A), R¹ is hydrogen, C₁-C₈ alkyl or C₂-C₈ alkenyl, each of which is unsubstituted or substituted with one or more R^{1c}, and R^{1c} is independently halogen, hydroxy, oxo, or phenyl.

In some embodiments of Formula (I-A), R¹ is hydrogen or a group selected from the group consisting of:

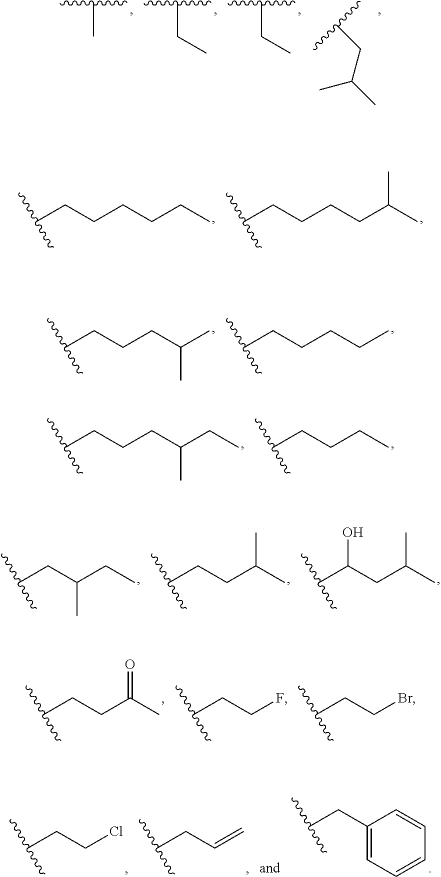

In some embodiments, disclosed herein, is a compound, or a pharmaceutically-acceptable salt or solvate thereof, having a structure represented by a structure of Formula (I-B):

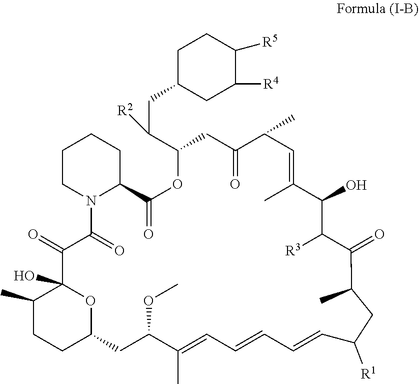

wherein,
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R₂₂, —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a1}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{l}a, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted; Raⁱ⁻ is C₁-C₈ alkyl, C₂-C₈ alkenyl, cycloalkyl, heterocycloalkyl, aryl, or heteroaryl;
   wherein the C₁-C₈ alkyl, cycloalkyl, heterocycloalkyl, aryl, and heteroaryl are
   each independently optionally substituted with one or more R²⁰;
or a conjugate of the compound of formula (I-B) with a biomolecule;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not —O(CH₂)₂OH—O(CH₂)₂OCH₂CH₃; —OC(═O)C(CH₂OH)₂CH₃; or OP(═O)(CH₃)₂.

In some embodiments of Formula (I-B), R¹ is hydrogen. In some embodiments of Formula (I-B), R¹ is CH₃. In some embodiments of Formula (I-B), R² is CH₃. In some embodiments of Formula (I-B), R³ is OCH₃. In some embodiments of Formula (I-A), R⁴ is OCH₃. In some embodiments of Formula (I-A), R⁴ is OH. In some embodiments of Formula (I-A), R⁵ is selected from the group consisting of:

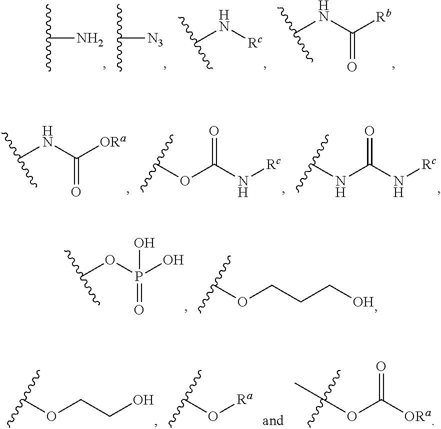

Exemplary compounds of the disclosure are depicted in Table 1.

TABLE 1
Exemplary structures of Formula (I)

| Compound | | |
|---|---|---|
| No | Structure | IUPAC Name |
| 5 | 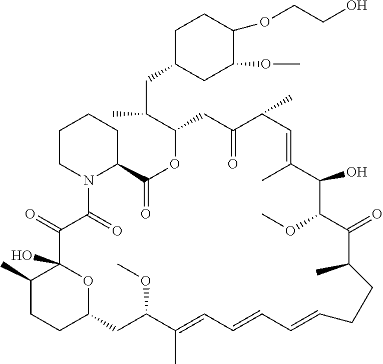 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CCC(OCCO)[C@H](OC)C2)CC(=O)[C@H](C)/C=C(\C)[C@@H](O)[C@@H](OC)C(=O)[C@H](C)CC/C=C/C=C/C=C/1C] | (3S,6R,7E,9R,10R,12R,15E,17E,19E,21S,23S,26R,27R, 34aS)-9,27-dihydroxy-3-((2R)-1-((1S,3R)-4-(2- hydroxyethoxy)-3-methoxycyclohexyl)propan-2-yl)- 10,21-dimethoxy-6,8,12,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 6 | 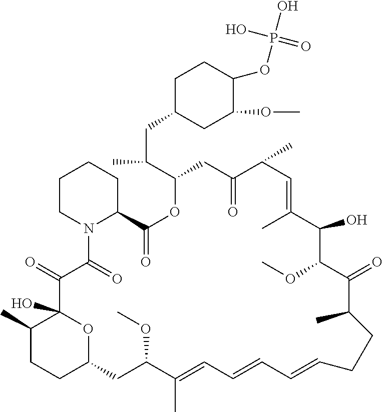 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CCC(OP(=O)(O)O)[C@H](OC)C2)CC(=O)[C@H](C)/C=C(\C)[C@@H](O)[C@@H](OC)C(=O)[C@H](C)CC/C=C/C=C/C=C/1C] | (2R,4S)-4-((R)-2- ((3S,6R,7E,9R,10R,12R,15E,17E,19E,21S,23S,26R, 27R,34aS)-9,27-dihydroxy-10,21-dimethoxy-6,8,12, 20,26-pentamethyl-1,5,11,28,29-pentaoxo- 1,4,5,6,9,10,11,12,13,14,21,22,23,24,25,26,27,28,29,31, 32,33,34,34a-tetracosahydro-3H-23,27-epoxypyrido[2,1- c][1]oxa[4]azacyclohentriacontin-3-yl)propyl)-2- methoxycyclohexyl dihydrogen phosphate |
| 7 | 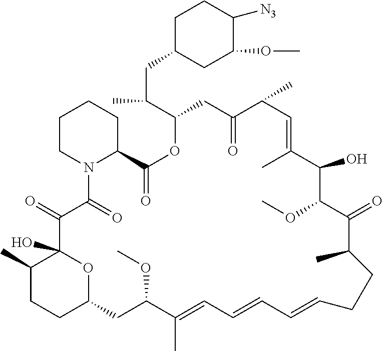 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CCC(N=[N+]=[N-])[C@H](OC)C2)CC(=O)[C@H](C)/C=C(\C)[C@@H](O)[C@@H](OC)C(=O)[C@H](C)CC/C=C/C=C/C=C/1C] | (3S,6R,7E,9R,10R,12R,15E,17E,19E,21S,23S,26R, 27R,34aS)-3-((2R)-1-((1S,3R)-4-azido-3- methoxycyclohexyl)propan-2-yl)-9,27-dihydroxy- 10,21-dimethoxy-6,8,12,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 9 | 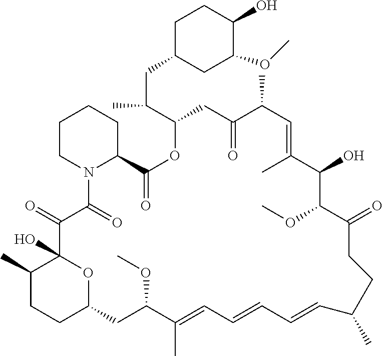 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)CC[C@H](C)/C=C/C=C/C=C/1C] | (3S,6R,7E,9R,10R,14S,15E,17E,19E,21S,23S,26R, 27R,34aS)-9,27-dihydroxy-3-((R)-1-((1S,3R,4R)-4- hydroxy-3-methoxycyclohexyl)propan-2-yl)-10,21- dimethoxy-6,8,14,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 10 | 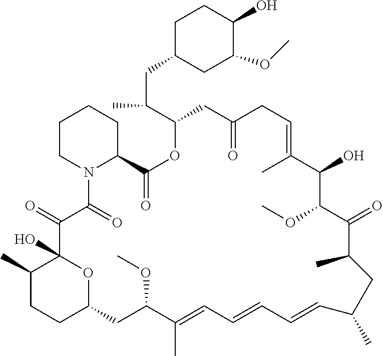 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)C/C=C(\C)[C@@H](O)[C@@H](OC)C(=O)[C@H](C)C[C@H](C)/C=C/C=C/C=C/1C] | (3S,7E,9R,10R,12R,14S,15E,17E,19E,21S,23S,26R, 27R,34aS)-9,27-dihydroxy-3-((R)-1-((1S,3R,4R)-4- hydroxy-3-methoxycyclohexyl)propan-2-yl)-10,21- dimethoxy-8,12,14,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 11 | 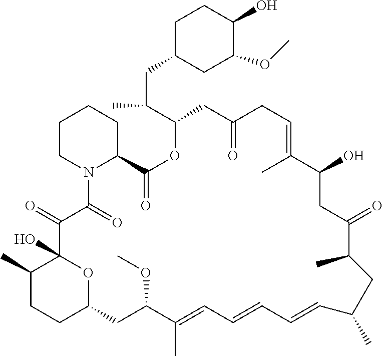 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)C/C=C(\C)[C@@H](O)CC(=O)[C@H](C)C[C@H](C)/C=C/C=C/C=C/1C] | (3S,7E,9S,12R,14S,15E,17E,19E,21S,23S,26R,27R, 34aS)-9,27-dihydroxy-3-((R)-1-((1S,3R,4R)-4-hydroxy- 3-methoxycyclohexyl)propan-2-yl)-21-methoxy- 8,12,14,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 12 | 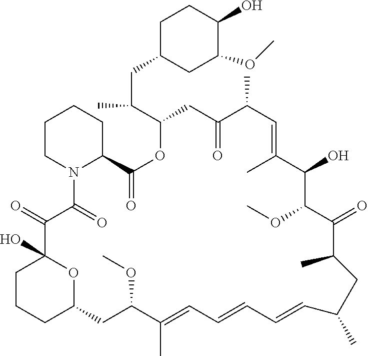 [SMILES: CO[C@H]1C[C@@H]2CCC[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] | (3S,6R,7E,9R,10R,12R,14S,15E,17E,19E,21S,23S,27R, 34aS)-9,27-dihydroxy-3-((R)-1-((1S,3R,4R)-4-hydroxy- 3-methoxycyclohexyl)propan-2-yl)-10,21-dimethoxy- 6,8,12,14,20-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 14 | 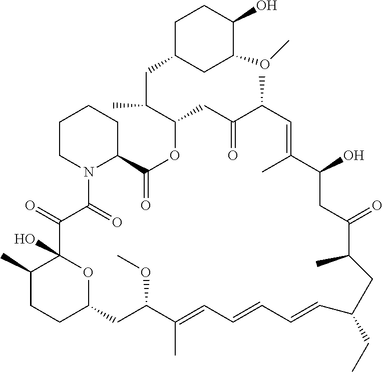 [SMILES: CC[C@@H]1/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)CC(=O)[C@H](C)C1] | (3S,6R,7E,9S,10R,12R,14S,15E,17E,19E,21S,23S,26R, 27R,34aS)-9,27-dihydroxy-3-((R)-1-((1S,3R,4R)-4- hydroxy-3-methoxycyclohexyl)propan-2-yl)-21- methoxy-6,8,10,12,14,20,26-heptamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 15 | 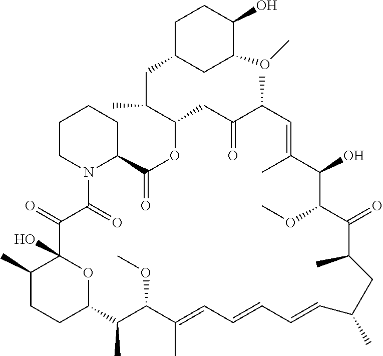 [SMILES: CO[C@@H]1/C(C)=C/C=C/C=C/[C@@H](C)C[C@@H](C)C(=O)[C@H](OC)[C@H](O)/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)[C@@H]1C] | (3S,6R,7E,9R,10R,12R,14S,15E,17E,19E,21S,22S,23S, 26R,27R,34aS)-9,27-dihydroxy-3-((R)-1-((1S,3R,4R)-4- hydroxy-3-methoxycyclohexyl)propan-2-yl)-10,21- dimethoxy-6,8,12,14,20,22,26-heptamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 16 | 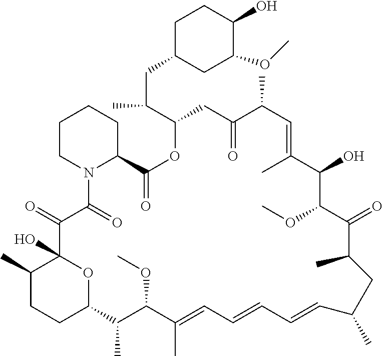 [SMILES: CO[C@@H]1/C(C)=C/C=C/C=C/[C@@H](C)C[C@@H](C)C(=O)[C@H](OC)[C@H](O)/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)[C@H]1C] | (3S,6R,7E,9R,10R,12R,14S,15E,17E,19E,21S,22R, 23S,26R,27R,34aS)-9,27-dihydroxy-3-((R)-1-((1S,3R, 4R)-4-hydroxy-3-methoxycyclohexyl)propan-2-yl)-10,21- dimethoxy-6,8,12,14,20,22,26-heptamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 17 | 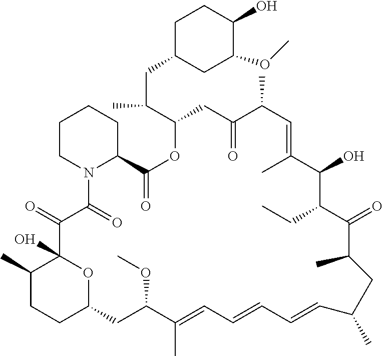 [SMILES: CC[C@H]1C(=O)[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@H]1O] | (3S,6R,7E,9S,10R,12R,14S,15E,17E,19E,21S,23S,26R, 27R,34aS)-10-ethyl-9,27-dihydroxy-3-((R)-1-((1S,3R, 4R)-4-hydroxy-3-methoxycyclohexyl)propan-2-yl)-21- methoxy-6,8,12,14,20,26-hexamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 18 | 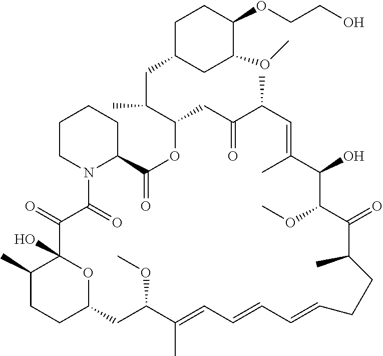 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)CC/C=C/C=C/C=C/1C] | (3S,6R,7E,9R,10R,12R,15E,17E,19E,21S,23S,26R,27R, 34aS)-9,27-dihydroxy-3-((R)-1-((1S,3R,4R)-4-(2- hydroxyethoxy)-3-methoxycyclohexyl)propan-2-yl)- 10,21-dimethoxy-6,8,12,20,26-pentamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |
| 19 | 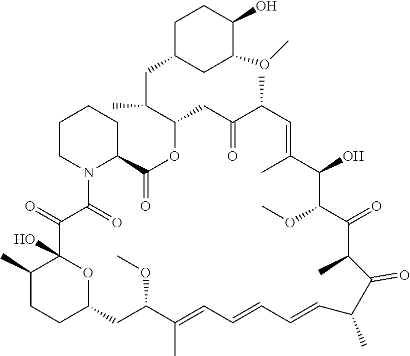 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C(=O)[C@H](C)/C=C/C=C/C=C/1C] | (3S,6R,7E,9R,10R,12R,14R,15E,17E,19E,21S,23S,26R, 27R,34aS)-9,27-dihydroxy-3-((R)-1-((1S,3R,4R)-4- hydroxy-3-methoxycyclohexyl)propan-2-yl)-10,21- dimethoxy-6,8,12,14,20,26-hexamethyl- 9,10,14,21,22,23,24,25,26,27,32,33,34,34a- tetradecahydro-3H-23,27-epoxypyrido[2,1- c][1]oxa[4]azacyclohentriacontine- 1,5,11,13,28,29(427,6H,12H,31H)-hexaone |
| 20 | 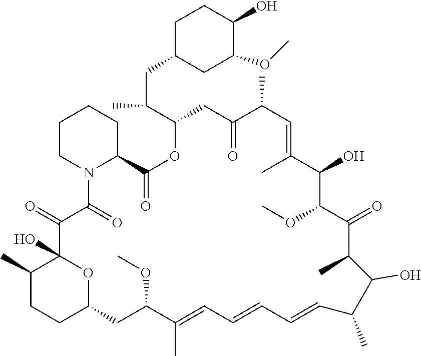 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C(O)[C@H](C)/C=C/C=C/C=C/1C] | (3S,6R,7E,9R,10R,12R,14R,15E,17E,19E,21S,23S,26R, 27R,34aS)-9,13,27-trihydroxy-3-((R)-1-((1S,3R,4R)-4- hydroxy-3-methoxycyclohexyl)propan-2-yl)-10,21- dimethoxy-6,8,12,14,20,26-hexamethyl- 9,10,12,13,14,21,22,23,24,25,26,27,32,33,34,34a- hexadecahydro-3H-23,27-epoxypyrido[2,1- c][1]oxa[4]azacyclohentriacontine- 1,5,11,28,29(4H,6H,31H)-pentaone |

### Conjugates

Provided herein is a conjugate, or a pharmaceutically-acceptable salt or solvate thereof, having a structure represented by a structure of Formula (I), or a pharmaceutically-acceptable salt thereof, to a biomolecule:

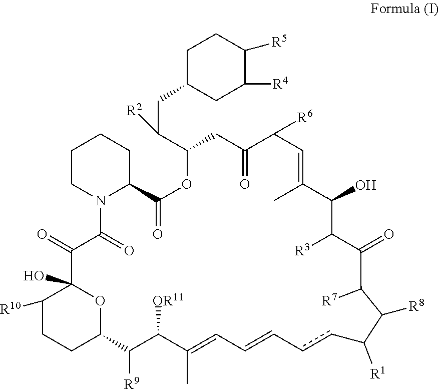

wherein,
[symbol] is a single or a double bond;
R¹ is hydrogen, halogen, —CN, OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)R^{a}, —OC(═O)—NR^{c}R^{d}, —NR^{c}R^{d}, —NR^{c}—C(═O)R^{b}, —NR^{c}—C(═O)R^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹—C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
(a) when R¹ is hydrogen, at least one of (i) R² is not CH₃, (ii) R³ is not OCH₃, or (iii) R⁴ is not —OCH₃; and
(b) when Rⁱ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

Provided herein is a conjugate, or a pharmaceutically-acceptable salt or solvate thereof, having a structure represented by a structure of Formula (I′), or a pharmaceutically-acceptable salt thereof, to a biomolecule:

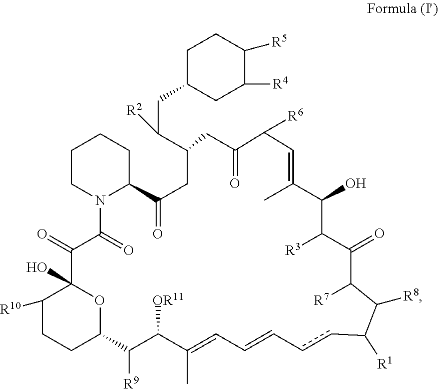

wherein,
[symbol] is a single or a double bond;
R¹ is hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R² is hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R³ is hydrogen, halogen, —OCH₃, —CN, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R¹¹ is hydrogen, alkyl, aryl or heteroaryl, wherein the alkyl, aryl and heteroaryl are each independently optionally substituted;
each R^{a}, R^{b}, R^{c}, and R^{d} is independently hydrogen, alkyl, aryl or heteroaryl, wherein the alkyl, aryl and heteroaryl are each independently optionally substituted;
or R^{c} and R^{d} are taken together with the nitrogen atom to which they are attached to form a heterocycloalkyl which is optionally substituted;
or a conjugate of the compound of formula (I) to a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
(c) when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃, (vi) R⁴ is not —OCH₃; or
   (vii) R⁵ is not —OH or —OC(═O)R^{b}, wherein R^{b} is alkyl, aryl or heteroaryl, wherein the alkyl, aryl and heteroaryl are each independently optionally substituted.

Provided herein is a conjugate, or a pharmaceutically-acceptable salt or solvate thereof, having a structure represented by a structure of Formula (I-A), or a pharmaceutically-acceptable salt thereof, to a biomolecule:

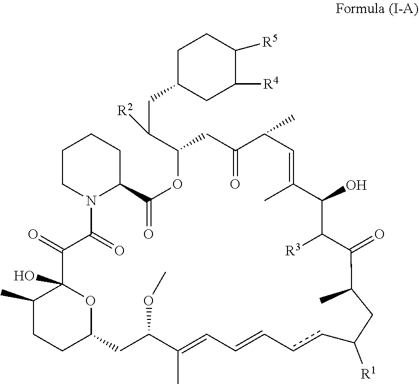

wherein,
[symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R₂₄, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, NR²¹, (═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH, or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR_{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one Rⁱa;
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

Provided herein is a conjugate, or a pharmaceutically-acceptable salt or solvate thereof, having a structure represented by a structure of Formula (I-B), or a pharmaceutically-acceptable salt thereof, to a biomolecule:

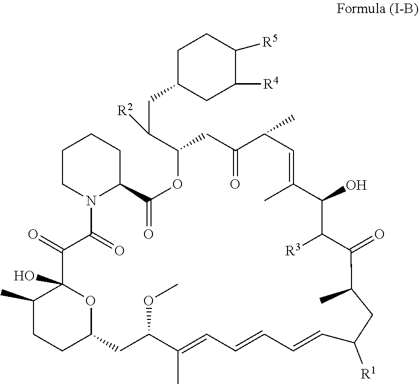

wherein,
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a1}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one lea;
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
R^{a1} is C₁-C₈ alkyl, C₂-C₈ alkenyl, cycloalkyl, heterocycloalkyl, aryl, or heteroaryl;
   wherein the C₁-C₈ alkyl, cycloalkyl, heterocycloalkyl, aryl, and heteroaryl are
   each independently optionally substituted with one or more R²⁰;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not —O(CH₂)₂OH —O(CH₂)₂OCH₂CH₃; —OC(═O)C(CH₂OH)₂CH₃; or OP(═O)(CH₃)₂.

In some embodiments, the conjugate comprises the compound of formula (I), (I′), (I-A) or (I-B) is bound directly to the biomolecule. In some embodiments, the comprises the compound of formula (I), (I′), (I-A) or (I-B) bound to the biomolecule or through a linker (L). In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody. In some embodiments, the conjugate comprises a plurality of compounds of formula (I), (I′), (I-A) or (I-B) conjugated to the biomolecule.

In some embodiments, the conjugate described herein comprises a ratio of compound (I), (I′), (I-A) or (I-B) to the biomolecule of 100 to 1. In some embodiments, the conjugate described herein comprises a ratio of compound (I), (I′), (I-A) or (I-B) to the biomolecule of 90 to 1. In some embodiments, the conjugate described herein comprises a ratio of compound (I), (I′), (I-A) or (I-B) to the biomolecule of 80 to 1. In some embodiments, the conjugate described herein comprises a ratio of compound (I), (I′), (I-A) or (I-B) to the biomolecule of 70 to 1. In some embodiments, the conjugate described herein comprises a ratio of compound (I), (I′), (I-A) or (I-B) to the biomolecule of 60 to 1. In some embodiments, the conjugate described herein comprises a ratio of compound (I), (I′), (I-A) or (I-B) to the biomolecule of 50 to 1. In some embodiments, the conjugate described herein comprises a ratio of compound (I), (I′), (I-A) or (I-B) to the biomolecule of 40 to 1. In some embodiments, the conjugate described herein comprises a ratio of compound (I), (I′), (I-A) or (I-B) to the biomolecule of 30 to 1. In some embodiments, the conjugate described herein comprises a ratio of compound (I), (I′), (I-A) or (I-B) to the biomolecule of 20 to 1. In some embodiments, the conjugate described herein comprises a ratio of compound (I), (I′), (I-A) or (I-B) to the biomolecule of 10 to 1. In some embodiments, the conjugate described herein comprises a ratio of compound (I), (I′), (I-A) or (I-B) to the biomolecule of 5 to 1. In some embodiments, the ratio of compound (I), (I′), (I-A) or (I-B) to the biomolecule is 4:1. In some embodiments, the ratio of compound (I), (I′), (I-A) or (I-B) to the biomolecule is 3:1. In some embodiments, the ratio of compound (I), (I′), (I-A) or (I-B) to the biomolecule is 2:1. In some embodiments, the ratio of compound (I), (I′), (I-A) or (I-B) to the biomolecule is 1:1.

In some embodiments, the conjugate described herein comprises a ratio of compound (I), (I′), (I-A) or (I-B) to the biomolecule of 5 to 1. In some embodiments, the ratio of compound (I), (I′), (I-A) or (I-B) to the biomolecule is 4:1. In some embodiments, the ratio of compound (I), (I′), (I-A) or (I-B) to the biomolecule is 3:1. In some embodiments, the ratio of compound (I), (I′), (I-A) or (I-B) to the biomolecule is 2: 1 . In some embodiments, the ratio of compound (I), (I′), (I-A) or (I-B) to the biomolecule is 1:1.

Provided herein is a conjugate, or a pharmaceutically-acceptable salt or solvate thereof, having a structure represented by a structure of Formula (I-C):

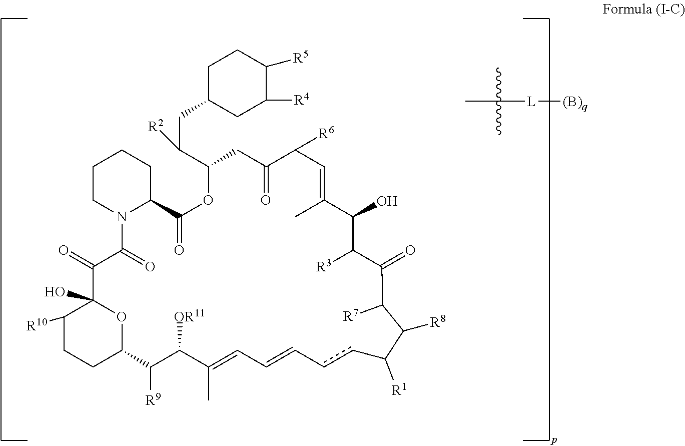

wherein,
[symbol] is a single or a double bond;
each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²³S(═O)₂NR²³R²⁴, —NR²¹C(═O)R₂₂, —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{id};
each R^{1a}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{b}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
B is a biomolecule;
p is 1-100; and
q is 1-100;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody.

In some embodiments, the linker L is attached to R¹, R², R³, R⁴, R⁵, R⁶, R⁷, R⁸, R⁹ R¹⁰ or R¹¹. In some embodiments, the linker L is attached to R¹, R², R³, R⁴, or R⁵. In some embodiments, the linker L is attached to In some embodiments, the linker L is attached to R². In some embodiments, the linker L is attached to R³. In some embodiments, the linker L is attached to R⁴. In some embodiments, the linker L is attached to R⁵. In some embodiments, the linker L is attached to R⁶. In some embodiments, the linker L is attached to R⁷. In some embodiments, the linker L is attached to R⁸. In some embodiments, the linker L is attached to R⁹. In some embodiments, the linker L is attached to R¹⁰. In some embodiments, the linker L is attached to

In some embodiments, p is 1-90. In some embodiments, p is 1-80. In some embodiments, p is 1-70. In some embodiments, p is 1-60. In some embodiments, p is 1-50. In some embodiments, p is 1-45. In some embodiments, p is 1-40. In some embodiments, p is 1-35. In some embodiments, p is 1-30. In some embodiments, p is 1-25. In some embodiments, p is 1-20. In some embodiments, p is 1-15. In some embodiments, p is 1-10. In some embodiments, p is 1-5. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16. In some embodiments, p is 17. In some embodiments, p is 18. In some embodiments, p is 19. In some embodiments, p is 20.

In some embodiments, q is 1. In some embodiments, q is 2. In some embodiments, q is 3. In some embodiments, q is 4. In some embodiments, q is 5.

In some embodiments, the ratio of p to q is 1:1 to 5:1. In some embodiments, the ratio of p to q is 5:1. In some embodiments, the ratio of p to q is 4:1. In some embodiments, the ratio of p to q is 3:1. In some embodiments, the ratio of p to q is 2:1. In some embodiments, the ratio of p to q is 1:1. In some embodiments, p is 5 and q is 1. In some embodiments, p is 4 and q is 1. In some embodiments, p is 3 and q is 1. In some embodiments, p is 2 and q is 1. In some embodiments, p is 1 and q is 1.

Provided herein is an antibody-drug conjugate (ADC) having a structure represented by a structure of Formula (I-D):

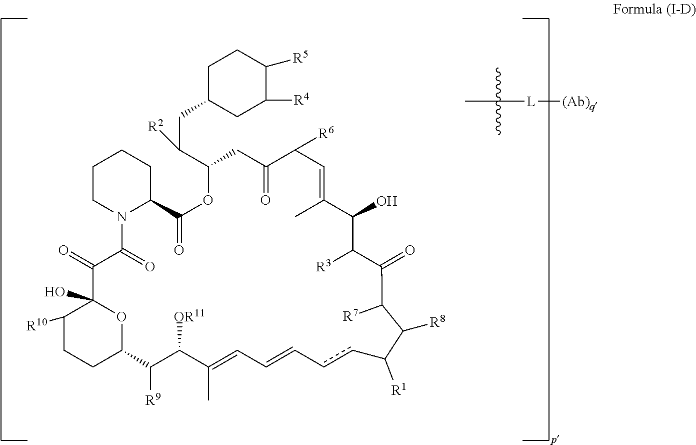

wherein,
[symbol] is a single or a double bond;
each R¹ is independently hydrogen, halogen, —CN, —OR²¹, —SR²¹, S(═O)R²², S(═O)₂R²², —NO₂, —NR²³R²⁴, —NR²¹S(═O)₂R²², —S(═O)₂NR²³R²⁴, —C(═)R²², —OC(═O)R²², OC(═O)R²², —C₁-C₈-alkyl-C(═O)R²⁰, —C(═O)C(═O)R²², —C(═O)OR²¹, —C(═O)NR²¹OR²¹, —NR²¹S(═O)²NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR₂₃R₂₄, —OC(═O)NR²³R²⁴, —NR²¹(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)N_{R}²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ independently is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, -01ta, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one Rⁱa; each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
Ab is an antibody or an antibody fragment;
p′ is 1-100; and
q′ is 1-100.

In some embodiments, when R¹ is hydrogen, at least one of (i) R² is not CH₃, (ii) R³ is not OCH₃, or (iii) R⁴ is not —OCH₃.

In some embodiments, when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

In some embodiments, the linker L is attached to R¹, R², R³, R⁴, R⁵, R⁶, R⁷, R⁸, R⁹ R¹⁰ or R¹¹. In some embodiments, the linker L is attached to R¹, R², R³, R⁴, or R⁵. In some embodiments, the linker L is attached to In some embodiments, the linker L is attached to R². In some embodiments, the linker L is attached to R³. In some embodiments, the linker L is attached to R⁴. In some embodiments, the linker L is attached to R⁵. In some embodiments, the linker L is attached to R⁶. In some embodiments, the linker L is attached to R⁷. In some embodiments, the linker L is attached to R⁸. In some embodiments, the linker L is attached to R⁹. In some embodiments, the linker L is attached to le° . In some embodiments, the linker L is attached to R¹¹.

In some embodiments, the biomolecule is selected from the group consisting of an antibody, an antibody fragment, an antigen, a nucleic acid, a nucleotide, a protein, a peptide, a peptide nucleic acid, a lipid and a carbohydrate. In some embodiments, the biomolecule is an antibody.

In some embodiments, p′ is 1-90. In some embodiments, p′ is 1-80. In some embodiments, p′ is 1-70. In some embodiments, p′ is 1-60. In some embodiments, p′ is 1-50. In some embodiments, p′ is 1-45. In some embodiments, p′ is 1-40. In some embodiments, p′ is 1-35. In some embodiments, p′ is 1-30. In some embodiments, p′ is 1-25. In some embodiments, p′ is 1-20. In some embodiments, p′ is 1-15. In some embodiments, p′ is 1-10. In some embodiments, p′ is 1-5. In some embodiments, p′ is 1. In some embodiments, p′ is 2. In some embodiments, p′ is 3. In some embodiments, p′ is 4. In some embodiments, p′ is 5. In some embodiments, p′ is 6. In some embodiments, p′ is 7. In some embodiments, p′ is 8. In some embodiments, p′ is 9. In some embodiments, p′ is 10. In some embodiments, p′ is 11. In some embodiments, p′ is 12. In some embodiments, p′ is 13. In some embodiments, p′ is 14. In some embodiments, p′ is 15. In some embodiments, p′ is 16. In some embodiments, p′ is 17. In some embodiments, p′ is 18. In some embodiments, p′ is 19. In some embodiments, p′ is 20.

In some embodiments, q′ is 1. In some embodiments, q′ is 2. In some embodiments, q′ is 3. In some embodiments, q′ is 4. In some embodiments, q′ is 5.

In some embodiments, the ratio of p′ to q′ is 1:1 to 5:1. In some embodiments, the ratio of p′ to q′ is 5:1. In some embodiments, the ratio of p′ to q′ is 4:1. In some embodiments, the ratio of p′ to q′ is 3:1. In some embodiments, the ratio of p′ to q′ is 2:1. In some embodiments, the ratio of p to q is 1:1. In some embodiments, p′ is 5 and q′ is 1. In some embodiments, p is 4′ and q′ is 1. In some embodiments, p is 3′ and q′ is 1. In some embodiments, p′ is 2 and q′ is 1. In some embodiments, p′ is 1 and q′ is 1.

In some embodiments, the conjugate of formula (I-C) or (I-D), or the compound of formula (I), (I′), (I-A) or (I-B), each conjugated to a biomolecule, is membrane-permeable.

As used herein, the term “biomolecule” and its variants comprise any compound isolated from a living organism, as well as analogs (including engineered and/or synthetic analogs), derivatives, mutants or variants and/or biologically active fragments of the same. For example, the biomolecule can be an oligonucleotide (e.g., DNA, RNA), protein or peptide (e.g., antibody, antigen), lipid, carbohydrate or other molecule found in biological entities (e.g., a peptide nucleic acid, a fatty acid, a vitamin, a cofactor, a purine, a pyrimidine, formysin, a phytochrome, a phytofluor, or phycobiliprotein, etc.) or entire biological entities (e.g., a virus, a phage, a prion a bacteria, etc.) or cells (e.g., eukaryotic or prokaryotic cells, etc.).

### Linkers

In some embodiments, a linker can link the compounds described herein to another molecule or chemical or biological structure. In some embodiments, a linker can link the compounds described herein to a biomolecule. Nonlimiting examples of a linker linking to another molecule or chemical or biological structure or biomolecule include a targeting moiety, a protein, drug precursor (e.g., prodrugs), a protein, a nucleic acid, or an antibody or fragments thereof. In some embodiments, a compound described herein, the compound can comprise a cleavable linker. In some embodiments, the linker is hydrolytically labile. In some embodiments, the linker is hydrolyzed by water. In some embodiments, the linker is hydrolyzed by an enzyme.

In some embodiments, the linker L comprises at least one group selected from the group consisting of a bond, alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene, is optionally substituted.

In some embodiments, the alkylene, alkenylene, alkynylene, cycloalkylene, arylene, heteroalkylene, heterocycloalkylene or heteroarylene are each independently substituted with one or more groups, each group being independently selected from the group consisting of —O—, —S—, silicone, amino, optionally substituted alkyl (e.g., alkoxy, haloalkyl) or optionally substituted cycloheteroalkylene (e.g, polyTHF).

In some embodiments, the linker L comprises at least one group selected from the group consisting of —O—, —S—, —NH—, —NH—(CH₂)ₘ—NH; —NH—(CH₂)ₘ—O; —O—(CH₂)ₘ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)ₙ—(C═O)—, —(C═O)—(CH═CH)ₙ—(C═O), —(C═O)—(OCH₂CH₂O)ₙ—(C═O)—, —O(CH₂CH₂O)ₙ—, and —(C═O)—(CH₂CH₂₀)ₙ—, or —(CH(CH₃)C(═O)O)ₙ—, wherein n is 1-20 and m is 1-20.

In some embodiments, the linker L is or comprises at least one amino acid. In some embodiments, the linker L is or comprises two amino acids. In some embodiments, the linker L is or comprises three amino acids. In some embodiments, the linker L comprises at least one of valine, citrulline, valine-citrulline, glutamic acid-valine-citrulline, maleimidocaproylvaline-citrulline (mcValCit), lysine-valine-citrulline (LysValCit), N-acetyl-lysine-valine-citrulline (AcLysValCit), p-aminobenzoic acid (PABA), p-aminocarbamate (PABC), diaminoalkylene, 4-((2S)-2-((2S)-2-(6-(3-mercapto-2,5-dioxopyrrolidin-1-yl)hexanamido)-3-methylbutanamido) ureidopentanamido)benzyl (2-aminoethyl)carbamate, or ((S)-5-acetamido-6-(((S)-1-(((S)-1-((4-((((2-aminoethyl)carbamoyl)oxy)methyl)phenyl)amino)-1-oxo-5-ureidopentan-2-yl)amino) methyl-1-oxobutan-2-yl)amino)-6-oxohexyl)carbamic acid. In some embodiments, the linker L is a bond.

In some embodiments, a linker can be valine-citrulline (VC), maleimidocaproylvaline-citrulline (mcVC), or N-acetyllysinevaline-citrulline (AcLysVC), 4-((2S)-2-((2S)-2-(6-(3-mercapto-2,5 -di oxopyrroli din-1-yl)hexanami do)-3 -methylbutanami do)-5-ureidopentanamido)benzyl (2-aminoethyl)carbamate (AcLysVC-PACB), or ((S)-5-acetamido-6-(((S)-1-(((S)-1-((4-((((2-aminoethyl)carbamoyl)oxy)methyl)phenyl)amino)-1-oxo-5-ureidopentan-2-yl)amino)-3-methyl-1-oxobutan-2-yl)amino)-6-oxohexyl)carbamic acid (thiol-Pyrrolidine-AcLysVC-PACB). In some embodiments, the linker further comprises p-aminocarbamate (e.g., 4-aminobenzyl hydrogen carbonate), diamine (e.g. ethylene diamine), one or more amino acids, or any combination thereof. In some embodiments, the linker can further comprise p-aminocarbamate (e.g., 4-aminobenzyl hydrogen carbonate), diamine (e.g. ethylene diamine), thiol, pyrrolidine, or one or more amino acids, or any combination thereof for increase stability.

In some embodiments, the linker comprises at least one oxo. In some embodiments, the linker is oxo. In some embodiments, the linker comprises at least one carbamate. In some embodiments, the linker is a carbamate. In some embodiments, the linker comprises at least one ester. In some embodiments, the linker is an ester. In some embodiments, the linker is or comprises a polyethylene glycol (PEG) or polypropylene glycol (PPG) linker. In some embodiments, the linker is or comprises a thiol (—S—).

The linker may be attached to any one or more positions of the compound of formula (I), (I′), (I-A) or (I-B). In some embodiments, the linker L is attached to R¹, R², R³, R⁴, R⁵, R⁶, R⁷, R⁸, R⁹, R¹⁰ or R¹¹. In some embodiments, the linker L is attached to R¹, R², R³, R⁴, or R⁵. In some embodiments, the linker L is attached to R¹. In some embodiments, the linker L is attached to R⁵.

The linker can be joined to any group in the compound of formula (I), (I′), (I-A) or (I-B) that is amenable to substitution. For example, linkage through a hydroxyl forms an ether or ester; linkage through a thiol forms a thioether or thioester; linkage through a carboxylate forms an ester, anhydride, or carbonate; linkage through an amino forms an amide, carbamate or thiocarbamate, and the like.

TABLE 2
Exemplary Linkers and Structure.

| Linker | | |
|---|---|---|
| Name | Base structure of Linker | Conjugated structure of Linker |
| VC | 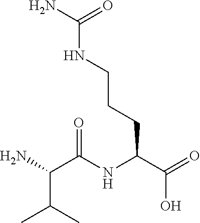 [SMILES: CC(C)[C@H](N)C(=O)N[C@@H](CCCNC(N)=O)C(=O)O] | 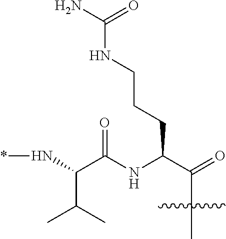 [SMILES: *N[C@H](C(=O)N[C@@H](CCCNC(N)=O)C(*)=O)C(C)C] |
| AcLysVC | 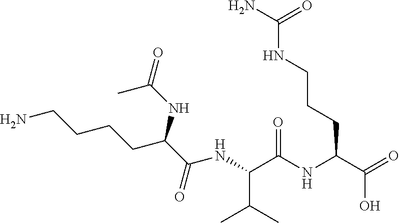 [SMILES: CC(=O)N[C@H](CCCCN)C(=O)N[C@H](C(=O)N[C@@H](CCCNC(N)=O)C(=O)O)C(C)C] | 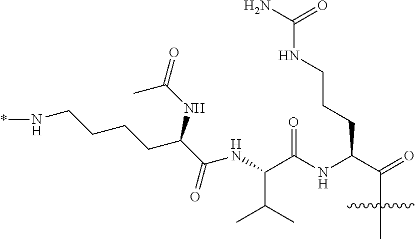 [SMILES: *NCCCC[C@@H](NC(C)=O)C(=O)N[C@H](C(=O)N[C@@H](CCCNC(N)=O)C(*)=O)C(C)C] |
| mcVC | 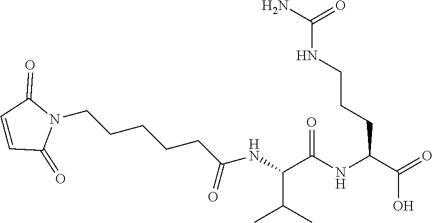 [SMILES: CC(C)[C@H](NC(=O)CCCCCN1C(=O)C=CC1=O)C(=O)N[C@@H](CCCNC(N)=O)C(=O)O] | 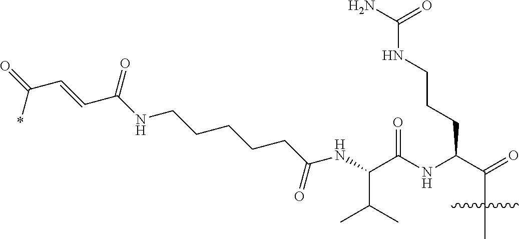 [SMILES: *C(=O)/C=C/C(=O)NCCCCCC(=O)N[C@H](C(=O)N[C@@H](CCCNC(N)=O)C(*)=O)C(C)C] |
| AcLysVC- PACB | 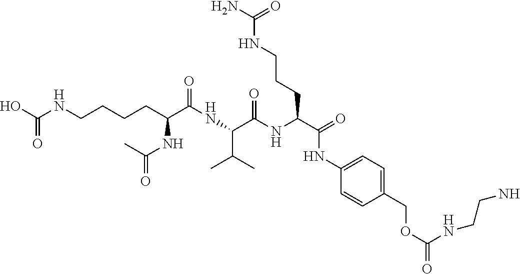 [SMILES: CC(=O)N[C@@H](CCCCNC(=O)O)C(=O)N[C@H](C(=O)N[C@@H](CCCNC(N)=O)C(=O)Nc1ccc(COC(=O)NCCN)cc1)C(C)C] | 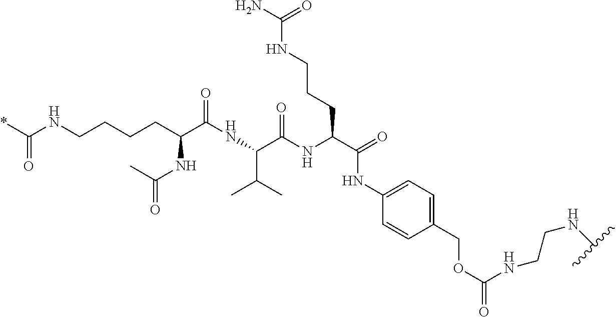 [SMILES: *NCCNC(=O)OCc1ccc(NC(=O)[C@H](CCCNC(N)=O)NC(=O)[C@@H](NC(=O)[C@H](CCCCNC(*)=O)NC(C)=O)C(C)C)cc1] |
| Thiol- Pyrrolidine- AcLysVC- PACB | 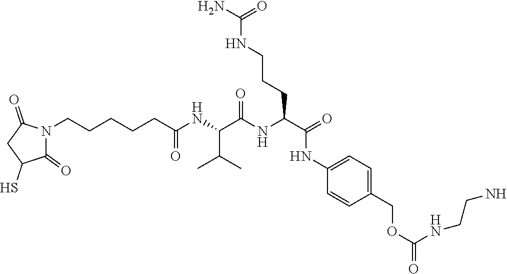 [SMILES: CC(C)[C@H](NC(=O)CCCCCN1C(=O)CC(S)C1=O)C(=O)N[C@@H](CCCNC(N)=O)C(=O)Nc1ccc(COC(=O)NCCN)cc1] | 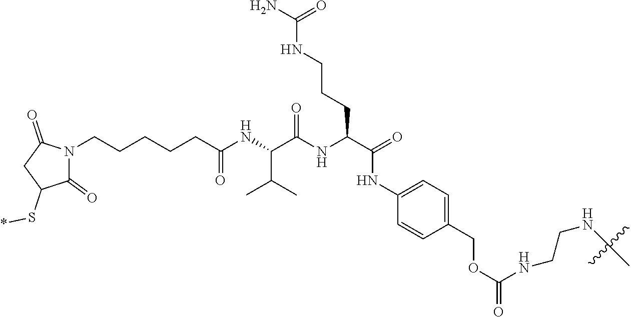 [SMILES: *NCCNC(=O)OCc1ccc(NC(=O)[C@H](CCCNC(N)=O)NC(=O)[C@@H](NC(=O)CCCCCN2C(=O)CC(S*)C2=O)C(C)C)cc1] |
| PABA | 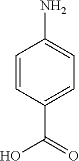 | 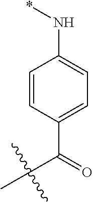 [SMILES: *Nc1ccc(C(*)=O)cc1] |
| PABC | 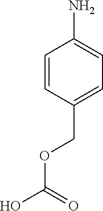 [SMILES: Nc1ccc(COC(=O)O)cc1] | 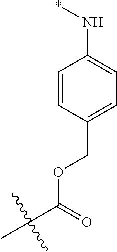 [SMILES: *Nc1ccc(COC(*)=O)cc1] |

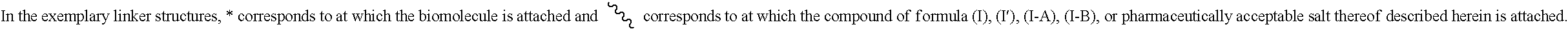

TABLE 3
Exemplary structures of Formula (I-C) or (I-D).

| Com- | |
|---|---|
| pound | |
| No | Structure |
| X-1 | 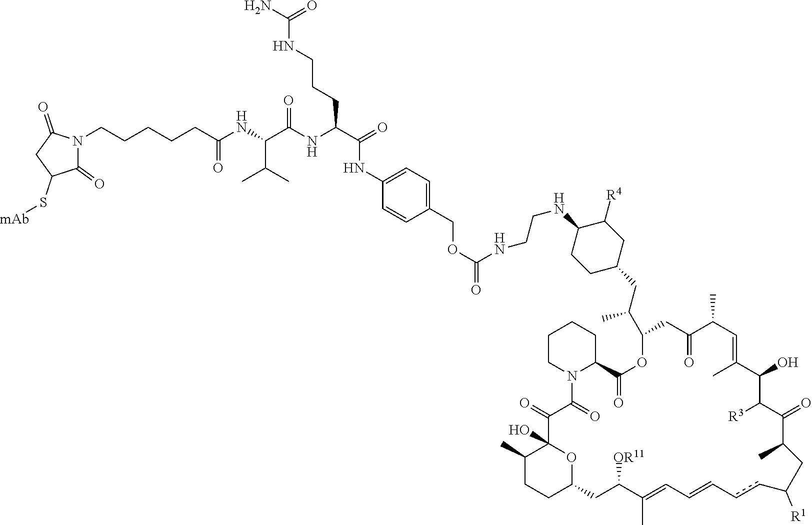 |
| X-2 | 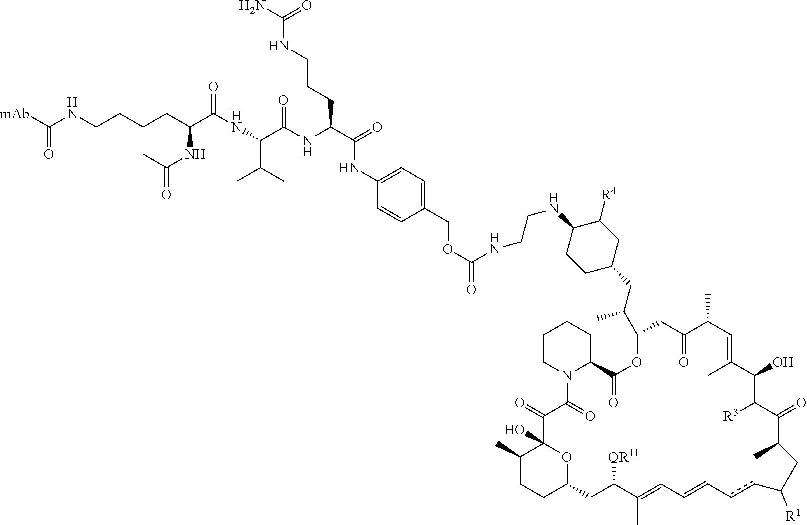 |
| X-3 | 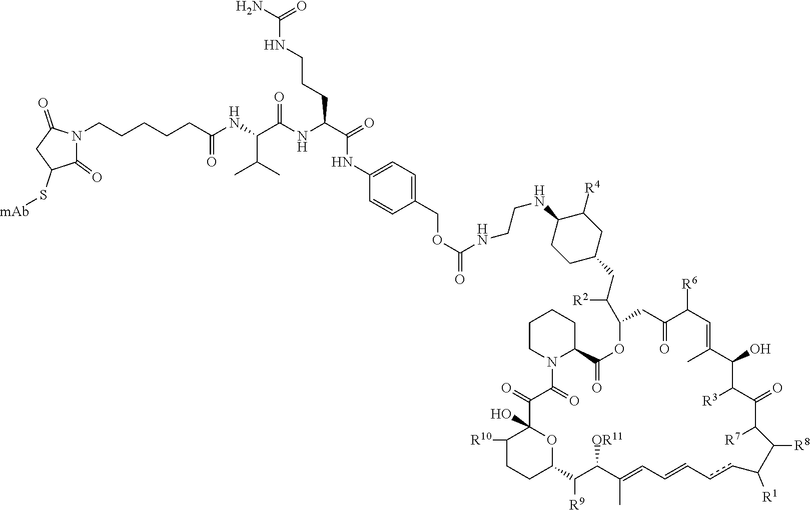 |
| X-4 | 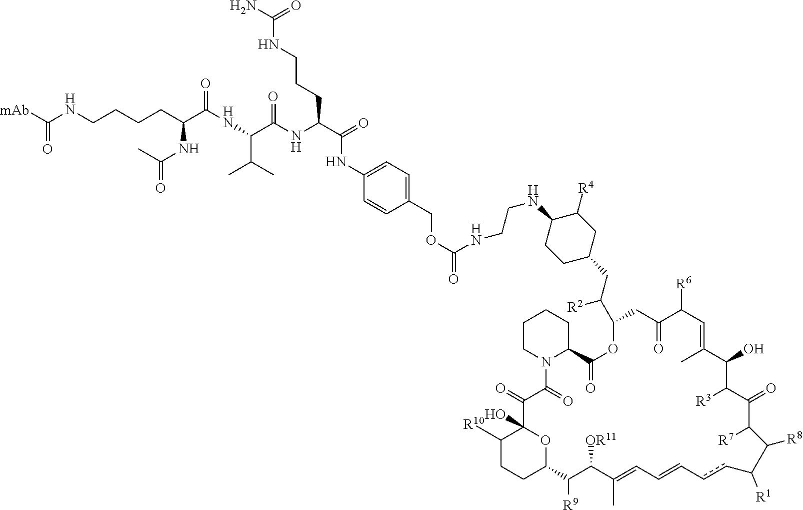 |

mAb represents monoclonal antibody

### Pharmaceutical Compositions

In some embodiments, the compound a described herein or a conjugate described herein can be formulated in a pharmaceutical composition comprising least one pharmaceutically-acceptable excipient. In some embodiments, the compound a described herein or a conjugate described herein can be formulated as an anti-aging pharmaceutical composition comprising at least one pharmaceutically-acceptable excipient.

In some embodiments, the pharmaceutical composition described herein can be formulated as a unit dosage form. In some embodiments, the pharmaceutical composition described herein can be formulated as a form suitable for oral, parenteral, rectal, ocular, intravenous or otic administration, or administration by inhalation.

### Pharmaceutically-Acceptable Salts

Provided herein are pharmaceutically-acceptable salts of the compounds described herein. As used herein, a pharmaceutically-acceptable salt includes, but is not limited to, acid addition salts or basic addition salts. Pharmaceutically-acceptable salts include, but are not limited to, alkali metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Examples of pharmaceutically-acceptable salts include, but are not limited to, bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorohydrate, and sulfate pentahydrate. Other representative pharmaceutically-acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3 -naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically-acceptable salt.

### Excipients

In some embodiments, a pharmaceutical composition can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N- acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

The binders that can be used in a pharmaceutical composition can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical composition can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical composition can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical composition can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes or their corresponding lakes.

In some instances, a pharmaceutical composition can comprise anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding (e.g., matrix binders (dry starch, dry sugars), film binders (PVP, starch paste, celluloses, bentonite, sucrose)), chemical binders (e.g., polymeric cellulose derivatives, such as carboxy methyl cellulose, HPC, HPMC, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), bufferants, where the acid is a pharmaceutically-acceptable acid, (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc) and where the base is a pharmaceutically-acceptable base (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine), or a pharmaceutically-acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants (e.g., titanium dioxide, food dyes, lakes, natural vegetable colorants, iron oxides, silicates, sulfates, magnesium hydroxide and aluminum hydroxide), coolants (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents (e.g., magnesium oxide), flavorants or desensitizers (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents), (e.g., sugars, polyvinylpyrrolidone, cellulosics, polymers and alginates).

In some instances, a pharmaceutical composition can comprise proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar).

In some instances, a pharmaceutical composition can comprise adsorbents. Many adsorbents are solid, porous or super porous adsorption materials. They comprise numerous micro- or nano- pores within their structures, resulting in very large surface areas, for example, greater than 500 m²/g. Exemplary absorbents include, without limitation, silica, active carbon, magnesium aluminum silicate, and diatomite.

In some embodiments, a compound described herein can be present in the form of a prodrug. The term “prodrug” as used herein, can refer to a drug precursor that, following administration to a subject and subsequent absorption, can be converted to an active, or a more active species via some process, such as conversion by a metabolic pathway. Thus, the term can encompass a derivative, which, upon administration to a recipient, can be capable of providing, either directly or indirectly, a compound, salt or a metabolite thereof. Some prodrugs can have a chemical group present on a prodrug that renders it less active and/or confers solubility or some other property to the drug. Once the chemical group has been cleaved and/or modified from the prodrug the active drug can be generated. Prodrugs can increase the bioavailability of a compound described herein when administered to a subject (e.g. by allowing an administered compound described herein to be more readily absorbed) or which enhance delivery of the compound described herein to a biological compartment (e.g. the brain or lymphatic system). In some embodiments, prodrugs include compounds where ester groups are bonded to any group that, when administered to a mammalian subject, cleaves to form a free hydroxyl. In some embodiments, compounds of the present disclosure are prodrugs comprising an ester group, wherein the ester group may be cleaved in-vivo to generate a compound having a hydroxyl group at the corresponding position.

### Administration

In some embodiments, a pharmaceutical formulation disclosed herein can be formulated into a variety of forms and administered by a number of different means. In some cases, a pharmaceutical formulation can be biodegradable. A pharmaceutical formulation can be administered orally, rectally, parenterally, ocular administration, topically, intravenously, otic administration, by inhalation administration, intranasally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intratracheal, intravascular, intravenous, intravitreal, epidural and subcutaneous, inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, intratracheal, or intraperitoneal injection. In some cases, an administering is a systemic administering. A systemic administering may be, for example, a parenteral injection at a site that allows for circulation.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. Tablets, pills, and the like can be compressed, multiply compressed, multiply layered, and/or coated. A coating can be single or multiple.

Liquid formulations can include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some cases, a liquid formulation can comprise a gel microsphere, or caulking hydrogel. In some instances, a combination of various formulations can be administered. In some embodiments, a tablet, pill, and the like can be formulated for an extended release profile.

Drops, such as eye drops or nose drops, may be formulated with one or more of a pharmaceutical composition in an aqueous or non-aqueous base also comprising one or more dispersing agents, solubilizing agents or suspending agents. Liquid sprays can be pumped or are conveniently delivered from pressurized packs. Drops can be delivered via a simple eye dropper-capped bottle, via a plastic bottle adapted to deliver liquid contents drop-wise, or via a specially shaped closure.

In some instances, a pharmaceutical composition described herein can be administered in a composition for topical administration. For topical administration, an active agent may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, aerosol formulations (e.g. sprays or foams), hydrogel, soaps, detergents, lotions or cakes of soap. Other conventional forms for this purpose include wound dressings, coated bandages or other polymer coverings, ointments, creams, lotions, pastes, jellies, sprays, and aerosols. Thus, a pharmaceutical composition disclosed herein can be delivered via patches or bandages for dermal administration.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. In some embodiments, a pharmaceutical composition can comprise the compound described herein and at least one excipient.

In some embodiments, the pharmaceutical composition described herein is in the form of a unit dose. In some embodiments, the pharmaceutical composition can be co-administered with a vaccine. In some embodiments, the pharmaceutical composition can be an adjuvant to a vaccine. In some embodiments, the pharmaceutical composition increases the efficacy and improve the effectiveness of a vaccine. In some embodiments, the pharmaceutical composition reduces the adverse effects of a vaccine.

### Methods of Treatment

### Neurodegenerative Diseases

In some embodiments, the pharmaceutical compositions described herein is administered to a subject in need thereof. In some embodiments, the subject in need thereof has a condition or disease. In some embodiments, the pharmaceutical composition described herein is administered to treat a subject in need thereof with a condition or disease, wherein the pharmaceutical composition herein reduces a symptom or symptoms of the condition or disease. In some embodiments, the condition or disease is a neurodegenerative disease.

Examples of neurodegenerative diseases include, but are not limited to, Alzheimer Disease, Parkinson Disease, Parkinson-like Disease, Huntington Disease, Lou Gehrig Disease, Multiple Sclerosis, autoimmune disorders, Pick Disease, diffuse Lewy body Disease, progressive supranuclear palsy (Steel-Richardson syndrome), multisystem degeneration (Shy-Drager syndrome), motor neuron diseases, amyotrophic lateral sclerosis, degenerative ataxias, cortical basal degeneration, ALS-Parkinson-Dementia complex of Guam, subacute sclerosing panencephalitis, synucleinopathies, primary progressive aphasia, striatonigral degeneration, Machado-Joseph disease/spinocerebellar ataxia type 3 and olivopontocerebellar degenerations, Gilles De La Tourette Disease, bulbar and pseudobulbar palsy, spinal and spinobulbar muscular atrophy (Kennedy Disease), primary lateral sclerosis, familial spastic paraplegia, Werdnig-Hoffmann Disease, Kugelberg-Welander Disease, Tay-Sach Disease, Sandhoff Disease, familial spastic disease, Wohlfart-Kugelberg-Welander Disease, spastic paraparesis, progressive multifocal leukoencephalopathy, prion diseases, including Creutzfeldt-Jakob Disease, Gerstmann-Straussler-Scheinker Disease, Kuru, and fatal familial insomnia.

Neurodegenerative diseases also include ischemic and hemorrhagic stroke, spinal cord injury, brain injury, Schizophrenia, Autism, Ataxia, Amyotrophic Lateral Sclerosis, Lou Gehrig's Disease, Lyme Disease, Meningitis, Migraine, Motor Neuron Diseases, Neuropathy, pain, brain damage, brain dysfunction, spinal cord disorders, peripheral nervous system disorders, cranial nerve disorders, autonomic nervous system disorders, seizure disorders, movement disorders, sleep disorders, headaches, lower back and neck pain, neuropathic pain, dementia, delirium and dementia dizziness and vertigo, stupor and coma, head injury, stroke, tumors of the nervous system, infections of the brain or spinal cord, prion diseases, depression, and drug addiction.

The term “dementia” as used herein refers to decline in cognitive function due to damage or disease in the brain or central nervous system beyond that which might be expected from normal aging. Dementias typically affect cognitive functions such as learning, memory, attention, language skills, and problem solving skills. Types and causes of dementia include, but are not limited to, Alzheimer's disease, vascular dementia (also known as multiinfarct dementia), Binswanger's disease, dementia with Lewy bodies (DLB), alcohol-induced persisting dementia, frontotemporal lobar degenerations (FTLD), Pick's disease, frontotemporal dementia (or frontal variant FTLD), semantic dementia (or temporal variant FTLD), progressive non-fluent aphasia, Creutzfeldt-lakob disease, Huntington's disease, Parkinson's di and AIDS dementia complex.

“Amyotrophic lateral sclerosis” or “ALS” are terms understood in the art and as used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

“Multiple sclerosis” or “MS” are terms understood in the art and as used herein to denote a progressive neurodegenerative disease resulting in destruction of the myelin covering of nerve cells, particularly of the brain and spinal cord. As used herein, “MS” includes all of the classifications of MS known in the art, including, but not limited Relapsing-remitting (RRMS) (typically characterized by partial or total recovery after attacks (also called exacerbations, relapses, or flares)), Secondary progressive (SPMS) (generally characterized by fewer relapses, with an increase in disability and symptoms), and Primary progressive (PPMS) (generally characterized by progression of symptoms and disability without remission).

“Alzheimer's disease” or “AD” are terms understood in the art and used herein to denote a progressive neurodegenerative disease characterized by dementia and defined by the American Psychiatric Association (in DSM IV) as the development of multiple cognitive deficits that includes memory impairment.

Parkinson's disease is a neurodegenerative disease. Many of the signs and symptoms associated with Parkinson's disease can precede typical Parkinson's disease, in some cases by many years. Involvement of the dopaminergic sub stantia nigra, which underlies the primary motor features of the disease, occurs at a time when the disease is well advanced at a neuropathological level, an observation that may account for the difficulties in successfully testing new drugs for potential disease modifying properties only after Parkinson's disease is evident. As a result, there is increasing interest in identifying pre-motor or prodromal signs and symptoms of Parkinson's disease in order to identify the disorder in its earliest stages, well before motor symptoms are in evidence. In one embodiment, a low-cost, non-invasive screening method is provided for pre-motor or prodromal Parkinson's disease. The motor features of Parkinson's disease are characterized by muscle rigidity, tremor, gait and postural abnormalities, a slowing of physical movement (bradykinesia) and, in extreme cases, a loss of physical movement (akinesia). The primary symptoms are the results of decreased stimulation of the motor cortex and other areas of the brain by the basal ganglia, normally caused by the insufficient formation and action of dopamine, which is produced in the dopaminergic neurons of the brain. The motor features of Parkinson's disease are just one component of a much more wide-spread disorder that causes an abundance of non-motor signs and symptoms, including olfactory dysfunction, REM sleep behavioral disorder (RBD), constipation, depression, and cognitive deficits. Many of these signs and symptoms can precede the motor symptoms by years to a decade or more.

Parkinson's-Like Diseases. There are several other conditions that have the features of Parkinson's disease and are interchangeably referred to as Parkinson's-like disease, secondary Parkinsonism, Parkinson's syndrome, or atypical Parkinson's. These are neurological syndromes that can be characterized by tremor, hypokinesia, rigidity, and postural instability. The underlying causes of Parkinson's-like disease are numerous, and diagnosis can be complex. A wide-range of etiologies can lead to a similar set of symptoms, including some toxins, a few metabolic diseases, and a handful of non-PD neurological conditions. A common cause is as a side effect of medications, mainly neuroleptic antipsychotics especially the phenothiazines (such as perphenazine and chlorpromazine), thioxanthenes (such as flupenthixol and zuclopenthixol) and butyrophenones (such as haloperidol (Haldol)), piperazines (such as ziprasidone), and rarely, antidepressants. Other causes include but are not limited to olivopontocerebellar degeneration, progressive supranuclear palsy, corticobasal degeneration, temporo-frontal dementia; drug induced like antipsychotics, prochlorperazine, metoclopromide; poisoning with carbon monoxide; head trauma; and Huntington's disease Parkinsonism. In some cases alpha-synucleinopathies can result in Parkinson's-like disease, secondary Parkinsonism, Parkinson's syndrome, or atypical Parkinson's. In a related embodiment the methods described herein are used to diagnose Parkinson's-like disease, secondary Parkinsonism, and Parkinson's syndrome.

### Viral Infections

In some embodiments, the condition or disease is a viral infection. In some embodiments, the pharmaceutical composition is effective to at least partially reduce a viral load of a coronavirus.

In some embodiments, the viral infection is caused by a coronavirus. Exemplary examples of coronavirus can be, but not limited to, Alphacoronavirus, Betacoronavirus, a Gammacoronavirus, Deltacoronavirus, 229E coronavirus, NL63 coronavirus, OC43 coronavirus, HKU1 coronavirus, middle east respiratory syndrome related coronavirus (MERS-CoV), severe acute respiratory syndrome coronavirus (SARS-CoV), severe acute respiratory syndrome coronavirus 2 (SARS-CoV-2 (COVID-19)), a mutated form of any of the forgoing, a variant of any of the foregoing, or any combination thereof.

In some embodiments, subject has or was previously diagnosed with a general symptom of a coronavirus. In some embodiments, general symptoms of a viral infection can be, but not limited to, a fever, a cough, a shortness of breath, breathing difficulties, or any combination thereof.

### Kits

Disclosed herein are kits. A kit can comprise a pharmaceutical composition described herein. In some aspects, a pharmaceutical composition can be packaged in a container. In some aspects, a kit can further comprise instructions that direct administration of a pharmaceutical composition to a subject. In some aspects, a kit can comprise a pharmaceutical composition disclosed herein and instructions for the use thereof. In some embodiments, the kit can comprise a compound as described herein or a conjugate as described herein, or a pharmaceutical composition as described herein. In some embodiments, the kit further comprises instructions for using the pharmaceutical composition. In some embodiments, the kit further comprising a coronavirus vaccine.

Methods of making a kit can include placing a pharmaceutical composition described herein in a container for packaging. A method can further comprise an inclusion of instructions for use. In some cases, instructions for use can direct administration of a unit dose of a pharmaceutical composition to a subject.

### Process

Described herein is a process for producing a biomolecule-drug-conjugate according to the present disclosure. In some embodiments, the process comprises conjugating a compound of formula (I), (I′), (I-A) or (I-B) as described herein to a biomolecule.

In some embodiments, the process comprises (a) providing a compound of formula (I), (I′), (I-A) or (I-B) as described herein; (b) linking a linker L to the compound of formula (I), (I′), (I-A) or (I-B) to obtain a conjugate; and (c) linking the conjugate of step (b) to a biomolecule so as to obtain a biomolecule-drug-conjugate.

In some embodiments, the process further comprising purifying the biomolecule-drug-conjugate. In some embodiments, the biomolecule is an antibody. In some embodiments, the conjugating is site specific on one or more engineered cysteine and/or glutamine residues on the antibody.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A compound, or a pharmaceutically-acceptable salt or solvate thereof, having a structure represented by a structure of Formula (I):

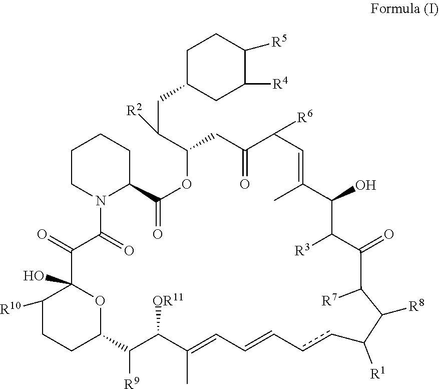

wherein,
[symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, NR²³R²⁴, —NR²¹S(═O)₂R²², —S(═O)₂NR²³R²⁴, —C(═O)R²², —OC(═O)R²², —C₁-C₈-alkyl-C(═O)R ²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈alkyl, C₂-C₈alkenyl, cycloalkyl, heterocycloalkyl, aryl, or heteroaryl; wherein the C₁-C₈alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;

each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of Formula (I) to a biomolecule;
wherein
   when R¹ is hydrogen, at least one of (i) R² is not CH₃, (ii) R³ is not OCH₃, or (iii) R⁴ is not —OCH₃;
   when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
   when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃;
   (vi) R⁴ is not —OCH₃; or (vii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

Embodiment 2. The compound of Embodiment 1, wherein R¹ is hydrogen.

Embodiment 3. The compound of Embodiment 1, wherein R¹ is CH₃.

Embodiment 4. The compound of any one of Embodiments 1-3, wherein R² is CH₃.

Embodiment 5. The compound of any one of Embodiments 1-4, wherein R³ is OCH₃.

Embodiment 6. The compound of any one of Embodiments 1-4, wherein R⁴ is OCH₃.

Embodiment 7. The compound of any one of Embodiments 1-6, wherein R⁴ is OH.

Embodiment 8. The compound of any one of the preceding Embodiments, wherein R⁵ is selected from the group consisting of:

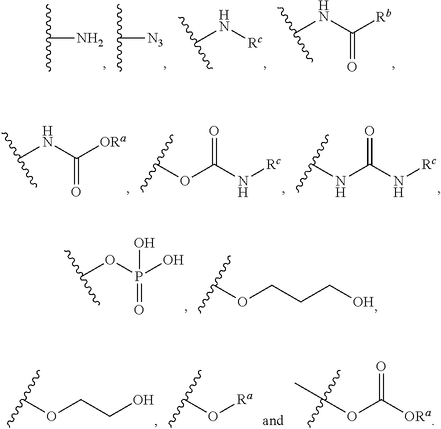

Embodiment 9. The compound of any one of the preceding Embodiments, wherein R⁶ is CH₃.

Embodiment 10. The compound of any one of the preceding Embodiments, wherein R⁷ is CH₃.

Embodiment 11. The compound of any one of the preceding Embodiments, wherein R⁸ is hydrogen.

Embodiment 12. The compound of any one of the preceding Embodiments, wherein R⁹ is hydrogen.

Embodiment 13. The compound of any one of the preceding Embodiments, wherein R¹⁰ is CH₃.

Embodiment 14. The compound of any one of the preceding Embodiments, wherein R¹¹ is CH₃.

Embodiment 15. The compound of any one of the preceding Embodiments, wherein R¹ is an optionally substituted alkyl.

Embodiment 16. The compound of any one of the preceding Embodiments, wherein R² is an optionally substituted alkyl.

Embodiment 17. The compound of any one of the preceding Embodiments, wherein R³ is an optionally substituted alkoxy.

Embodiment 18. The compound of any one of the preceding Embodiments, wherein R⁴ is OH or an optionally substituted alkoxy.

Embodiment 19. The compound of any one of the preceding Embodiments, wherein at least one of R¹, R⁶, R⁷, R⁸, R⁹ or R¹⁰ is hydrogen, C₁-C₈ alkyl or C₂-C₈ alkenyl, each of which is unsubstituted or substituted with one or more R^{1e}, wherein R^{1e} is independently halogen, hydroxy, oxo, or phenyl.

Embodiment 20. The compound of any one of the preceding Embodiments, wherein at least one of R¹, R⁶, R⁷, R⁸, R⁹ or R¹⁰ is hydrogen or a group selected from the group consisting of:

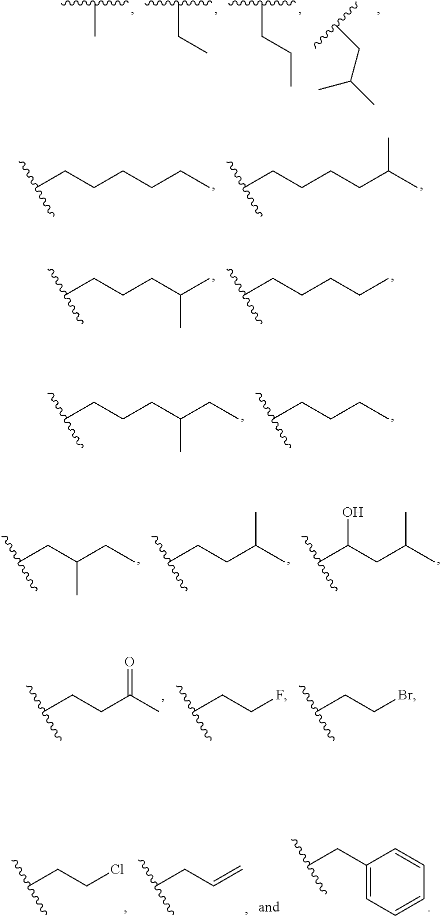

Embodiment 21. The compound of any one of the preceding Embodiments, wherein the compound is selected from group from the consisting of:

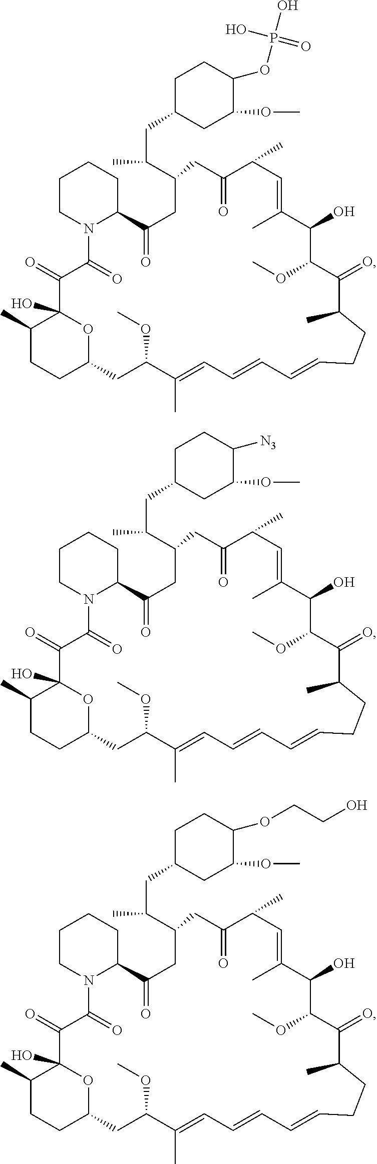

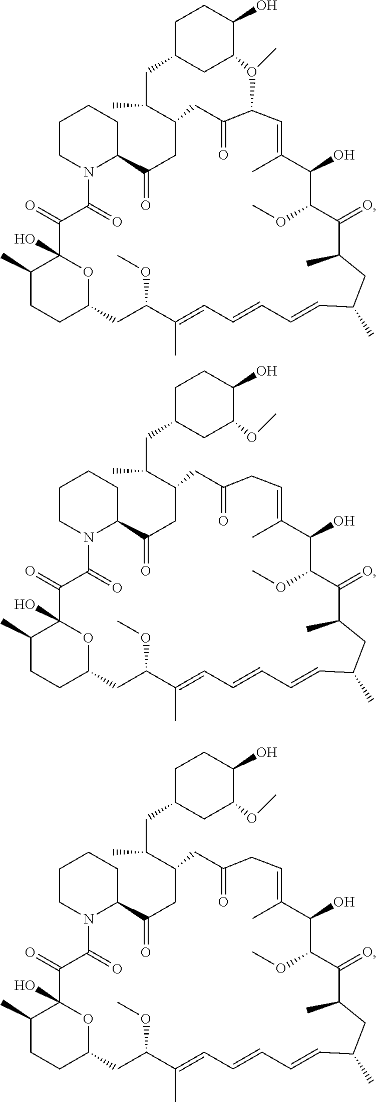

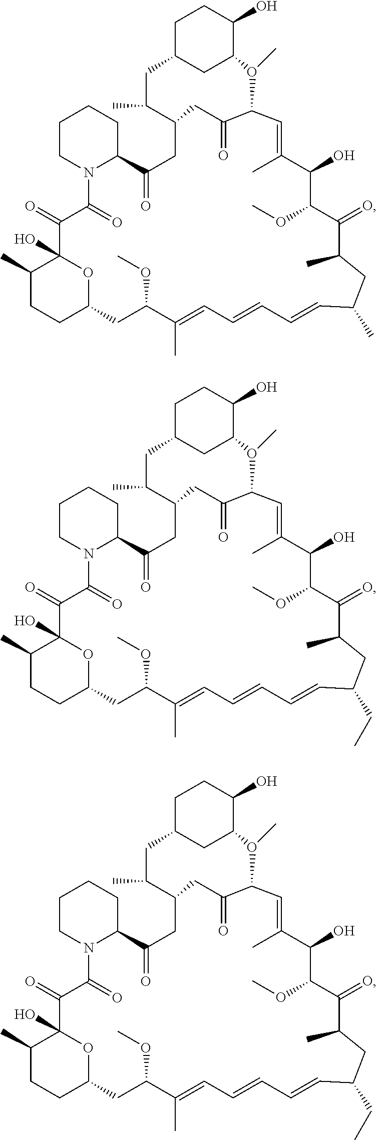

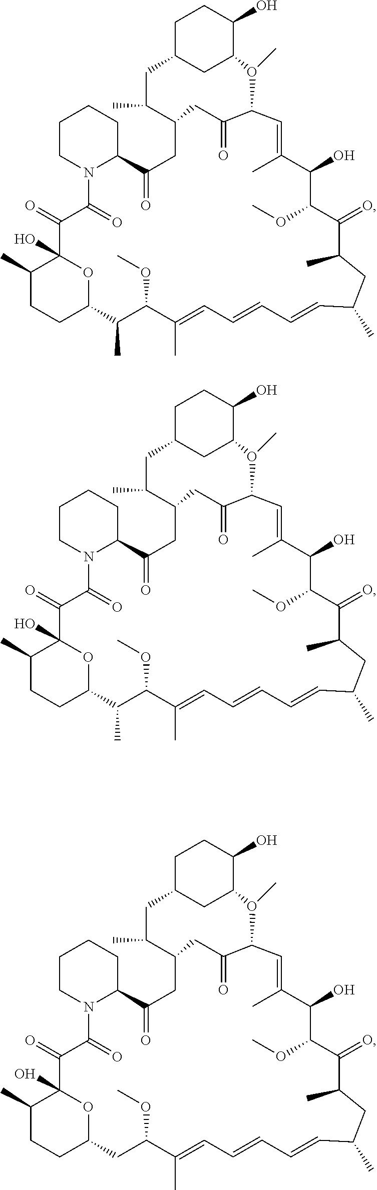

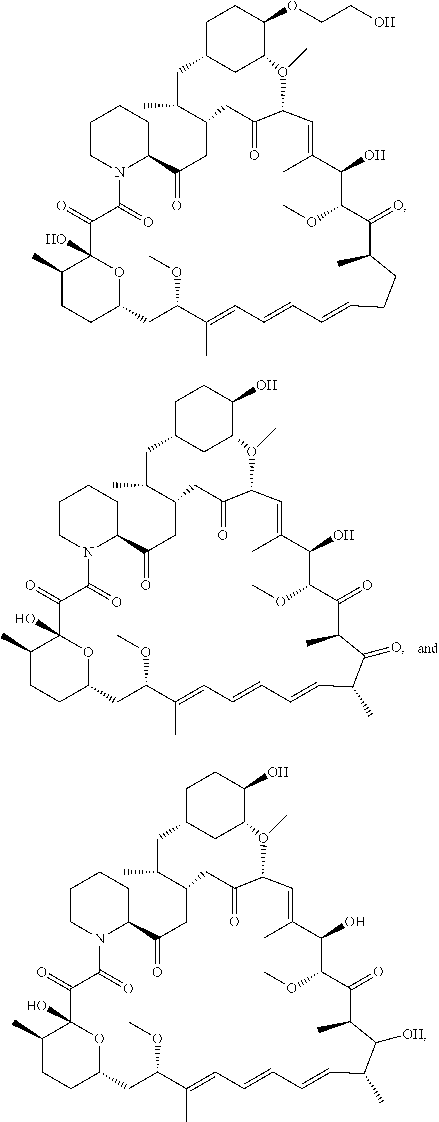

Embodiment 22. A compound, or a pharmaceutically-acceptable salt or solvate thereof, having a structure represented by a structure of Formula (I-A):

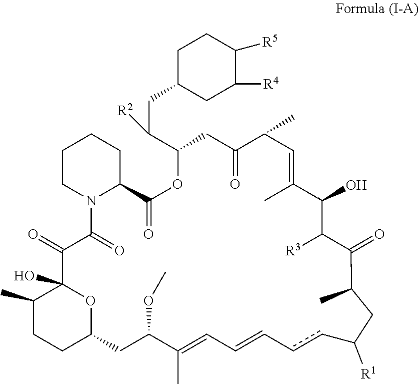

wherein,
[symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²², —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR₂₁S(═O)₂NR²³R²⁴, —NR²¹C(═O)_{R}²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH, or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of Formula (I-A) to a biomolecule;
wherein
   when R¹ is hydrogen, at least one of (i) R² is not CH₃, (ii) R³ is not OCH₃, or (iii) R⁴ is not —OCH₃;
   when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
   when R¹ is H or methyl, at least one of (i) R⁴ is not —OCH₃; or (ii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

Embodiment 23. The compound of Embodiment 22, wherein R¹ is hydrogen.

Embodiment 24. The compound of Embodiment 22, wherein R¹ is CH₃.

Embodiment 25. The compound of any one of Embodiments 22-24, wherein R² is CH₃.

Embodiment 26. The compound of any one of Embodiments 22-25, wherein R³ is OCH₃.

Embodiment 27. The compound of any one of Embodiments 22-26, wherein R⁴ is OCH₃.

Embodiment 28. The compound of any one of Embodiments 22-26, wherein R⁴ is OH.

Embodiment 29. The compound of any one of Embodiments 22-26, wherein R¹ is an optionally substituted alkyl.

Embodiment 30. The compound of any one of Embodiments 22-26, wherein R² is an optionally substituted alkyl.

Embodiment 31. The compound of any one of Embodiments 22-26, wherein R³ is an optionally substituted alkoxy.

Embodiment 32. The compound of any one of Embodiments 22-26, wherein R⁴ is an optionally substituted alkoxy.

Embodiment 33. The compound of any one of Embodiments 22-32, wherein R⁵ is selected from the group consisting of:

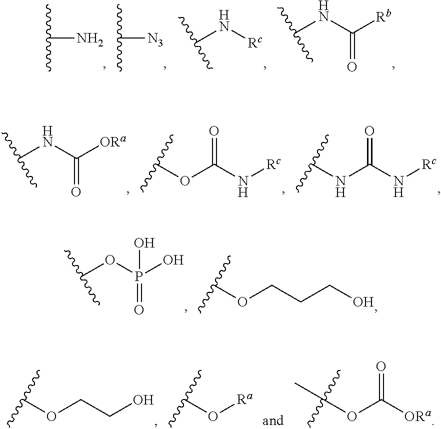

Embodiment 34. The compound of any one of Embodiments 22-33, wherein R¹ is hydrogen, C₁-C₈ alkyl or C₂-C₈ alkenyl, each of which is unsubstituted or substituted with one or more R^{1e}, wherein R^{1e} is independently halogen, hydroxy, oxo, or phenyl.

Embodiment 35. The compound of any one of Embodiments 22-34, wherein R¹ is hydrogen or a group selected from the group consisting of:

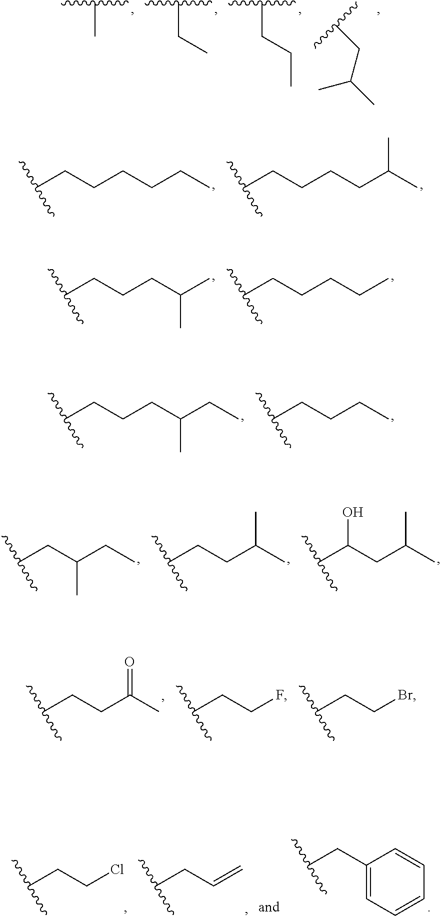

Embodiment 36. A compound, or a pharmaceutically-acceptable salt or solvate thereof, having a structure represented by a structure of Formula (I-B):

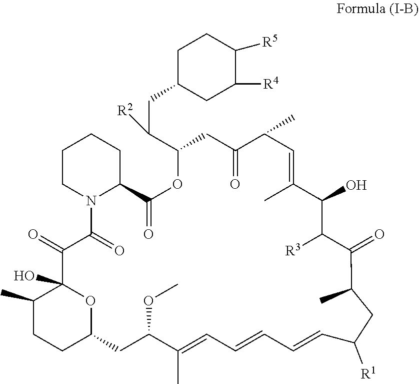

wherein,
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)_{NR}²³R²⁴, —NR²iₛ(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR¹, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{id};
each R^{1a}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
R^{a1} is C₁-C₈ alkyl, C₂-C₈ alkenyl, cycloalkyl, heterocycloalkyl, aryl, or heteroaryl; wherein the C₁-C₈ alkyl, cycloalkyl, heterocycloalkyl, aryl, and heteroaryl are each independently optionally substituted with one or more R²⁰;
or a conjugate of the compound of Formula (I-B) to a biomolecule;
wherein
   when R¹ is hydrogen, at least one of (i) R² is not CH₃, (ii) R³ is not OCH₃, or (iii) R⁴ is not —OCH₃; and
   when R¹ is CH₃, at least one of (i) R² is not CH₃, (ii) R³ is not hydrogen, —CH₃ or —OCH₃, (iii) R⁴ is not —OCH₃; or (iv) R⁵ not —O(CH₂)₂OH —O(CH₂)₂OCH₂CH₃; —OC(—O)C(CH₂OH)₂CH₃; or OP(═O)(CH₃)₂.

Embodiment 37. The compound of Embodiment 36, wherein R¹ is hydrogen.

Embodiment 38. The compound of Embodiment 36, wherein R¹ is CH₃.

Embodiment 39. The compound of any one of Embodiments 36-38, wherein R² is CH₃.

Embodiment 40. The compound of any one of Embodiments 36-39, wherein R³ is OCH₃.

Embodiment 41. The compound of any one of Embodiments 36-40, wherein R⁴ is OCH₃.

Embodiment 42. The compound of any one of Embodiments 36-40, wherein R⁴ is OH.

Embodiment 43. The compound of any one of Embodiments 36-40, wherein R¹ is an optionally substituted alkyl.

Embodiment 44. The compound of any one of Embodiments 36-40, wherein R² is an optionally substituted alkyl.

Embodiment 45. The compound of any one of Embodiments 36-40, wherein R³ is an optionally substituted alkoxy.

Embodiment 46. The compound of any one of Embodiments 36-40, wherein R⁴ is OH or an optionally substituted alkoxy.

Embodiment 47. The compound of any one of Embodiments 36-46, wherein R⁵ is selected from the group consisting of:

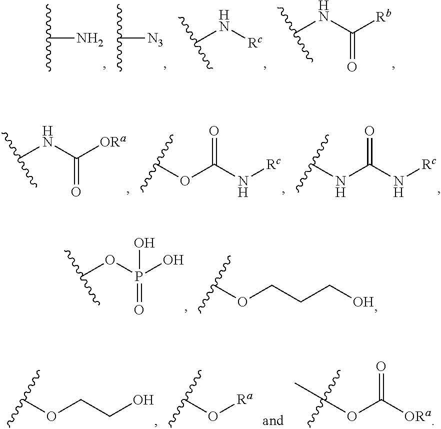

Embodiment 48. The compound of any one of anyone of the preceding Embodiments, wherein the biomolecule is selected from the group consisting of an antibody, an antibody fragment, an antigen, a nucleic acid, a nucleotide, a protein, a peptide, a peptide nucleic acid, a lipid and a carbohydrate.

Embodiment 49. The compound of any one of the preceding Embodiments, wherein the biomolecule is an antibody.

Embodiment 50. A conjugate of a compound of formula (I), or a pharmaceutically-acceptable salt thereof, to a biomolecule

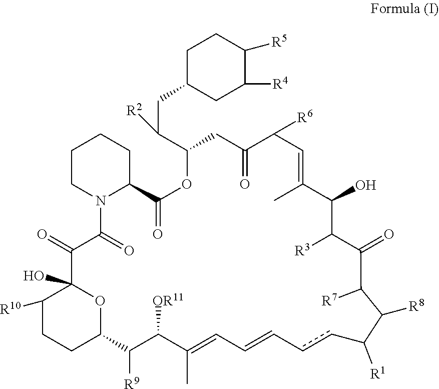

wherein,
[symbol] is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—(═O)R^{b}, —NR^{c}—C(═O)OR^{a}—NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl; each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
   when R¹ is hydrogen, at least one of (i) R² is not CH₃, (ii) R³ is not OCH₃, or (iii) R⁴ is not —OCH₃; and
   when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

Embodiment 51. The conjugate of Embodiment 50, wherein the compound of formula (I) is bound directly to the biomolecule.

Embodiment 52. The conjugate of Embodiment 50, wherein the compound of formula (I) is bound to the biomolecule or through a linker (L).

Embodiment 53. The conjugate of any one of Embodiments 50-52, wherein the biomolecule is selected from the group consisting of an antibody, an antibody fragment, an antigen, a nucleic acid, a nucleotide, a protein, a peptide, a peptide nucleic acid, a lipid and a carbohydrate.

Embodiment 54. The conjugate of any one of Embodiments 50-53, wherein the biomolecule is an antibody.

Embodiment 55. The conjugate of any one of Embodiments 50-54, comprising a plurality of compounds of formula (I) conjugated to the biomolecule.

Embodiment 56. The conjugate of Embodiment 55, wherein the ratio of compound (I) to the biomolecule is about 100 to about 1.

Embodiment 57. The conjugate of Embodiment 56, wherein the ratio of compound (I) to the biomolecule is 4.

Embodiment 58. The conjugate of Embodiment 56, wherein the ratio of compound (I) to the biomolecule is 2.

Embodiment 59. A conjugate, or a pharmaceutically-acceptable salt or solvate thereof, having a structure represented by a structure of Formula (I-C):

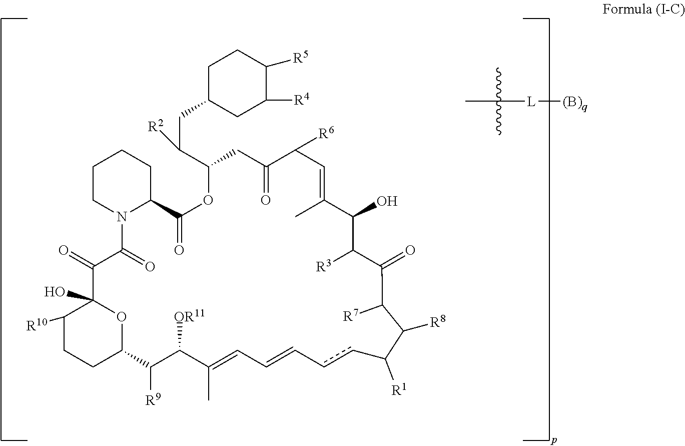

wherein,
[symbol] is a single or a double bond;
each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², ——NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —

NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;

R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
B is a biomolecule;
p is 1-100; and
q is 1-100;
wherein
   when R¹ is hydrogen, at least one of (i) R² is not CH₃, (ii) R³ is not OCH₃, or (iii) R⁴ is not —OCH₃; and
   when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃;
   —OC(═O)C(CH₂OH)₂CH₃; or OP(═O)(CH₃)₂.

Embodiment 60. The conjugate of Embodiment 59, wherein the linker L is attached to R¹, R², R³, R⁴, R⁵, R⁶, R⁷, R⁸, R⁹, R¹⁰ or R¹¹.

Embodiment 61. The conjugate of Embodiment 59, wherein the linker L is attached to R¹, R², R³, R⁴, or R⁵.

Embodiment 62. The conjugate of any one of Embodiments 59-61, wherein the linker L is attached to R¹.

Embodiment 63. The conjugate of any one of Embodiments 59-61, wherein the linker L is attached to R⁵.

Embodiment 64. The conjugate of any one of Embodiments 59-63, wherein the biomolecule is selected from the group consisting of an antibody, an antibody fragment, an antigen, a nucleic acid, a nucleotide, a protein, a peptide, a peptide nucleic acid, a lipid and a carbohydrate.

Embodiment 65. The conjugate of Embodiment 64, wherein the biomolecule is an antibody.

Embodiment 66. The conjugate of any one of Embodiments 59-65, wherein the ratio of p to q is 1:1 to 5:1.

Embodiment 67. The conjugate of Embodiment 66, wherein p is 4 and q is 1.

Embodiment 68. The conjugate of Embodiment 66, wherein p is 2 and q is 1.

Embodiment 69. An antibody-drug conjugate (ADC) having a structure represented by a structure of Formula (I-D):

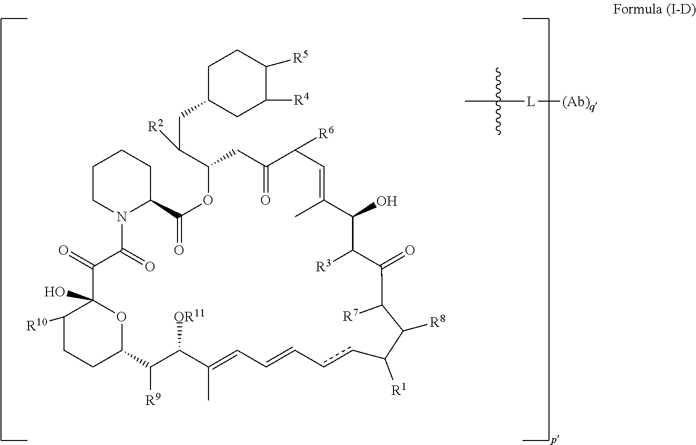

wherein,
[symbol] is a single or a double bond;
each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, Cₗ-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ independently is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
Ab is an antibody or an antibody fragment;
p′ is 1-100; and
q′ is 1-100.

Embodiment 70. The conjugate of Embodiment 69, wherein the linker L is attached to R¹, R², R³, R⁴, R⁵, R⁶, R⁷, R⁸, R⁹, R¹⁰ or R¹¹.

Embodiment 71. The conjugate of Embodiment 69, wherein the linker L is attached to R¹, R², R³, R⁴, or R⁵.

Embodiment 72. The conjugate of any one of Embodiments 69-71, wherein the linker L is attached to R¹.

Embodiment 73. The conjugate of Embodiment 69-71, wherein the linker L is attached to R⁵.

Embodiment 74. The conjugate of any one of Embodiments 69-73, wherein the ratio of p′ to q′ is 1:1 to 5:1.

Embodiment 75. The conjugate of Embodiment 74, wherein p′ is 4 and q′ is 1.

Embodiment 76. The conjugate of Embodiment 74, wherein p′ is 2 and q′ is 1.

Embodiment 77. The conjugate of any one of Embodiments 52-76, wherein the linker L comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

Embodiment 78. The conjugate of any one of Embodiments 52-76, wherein the linker L comprises at least one group selected from the group consisting of —O—, —S—, —NH—, —NH—(CH₂)ₘ—NH; —NH—(CH₂)ₘ—O; —O—(CH₂)ₘ—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)ₙ—(C═O)—, —(C═O)—(CH═CH)ₙ—(C═O), —(C═O)—(OCH₂CH₂₀)ₙ—(C═O)—, —O(CH₂CH₂₀), —(C═O)—(CH₂CH₂O)ₙ—, and —(CH(CH₃)C(═O)O)ₙ—, wherein n is 1-20 and m is 1-20.

Embodiment 79. The conjugate of any one of Embodiments 52-76, wherein the linker L is or comprises at least one amino acid.

Embodiment 80. The conjugate of any one of Embodiments 52-76, wherein the linker L is or comprises two amino acids.

Embodiment 81. The conjugate of any one of Embodiments 52-76, wherein the linker L is or comprises three amino acids.

Embodiment 82. The conjugate of any one of Embodiments 52-76, wherein the linker L comprises at least one of valine, citrulline, valine-citrulline, glutamic acid-valine-citrulline, maleimidocaproylvaline-citrulline (mcValCit), lysine-valine-citrulline (LysValCit), N-acetyl-lysine-valine-citrulline (AcLysValCit), p-aminobenzoic acid (PABA), p-aminocarbamate (PABC), diaminoalkylene, 4-((2S)-2-((2S)-2-(6-(3-mercapto-2,5-dioxopyrrolidin-1-yl)hexanamido)-3-methylbutanamido)-5-ureidopentanamido)benzyl (2-aminoethyl)carbamate, or ((S)-5-acetamido-6-(((S)-14(S)-14(4-((((2-aminoethyl)carbamoyl)oxy)methyl)phenyl)amino)-1-oxo-5-ureidopentan-2-yl)amino)-3-methyl-1-oxobutan-2-yl)amino)-6-oxohexyl)carbamic acid.

Embodiment 83. The conjugate of any one of Embodiments 52-76, wherein the linker L is a bond.

Embodiment 84. The conjugate of any one of Embodiments 50-83, wherein the compound of formula (I), (I-C) or (I-D) is membrane-permeable.

Embodiment 85. A pharmaceutical composition comprising a compound according to any one of Embodiments 1-49, or a conjugate of any one of Embodiments 50-84, and at least one pharmaceutically-acceptable excipient.

Embodiment 86. An anti-aging pharmaceutical composition a compound according to any one of Embodiments 1-49, or a conjugate of any one of Embodiments 50-84, and at least one pharmaceutically-acceptable excipient.

Embodiment 87. The pharmaceutical composition of Embodiment 84 or 86, wherein the pharmaceutical composition is in a unit dosage form.

Embodiment 88. The pharmaceutical composition according to any one of Embodiments 84-87, in a form suitable for oral, parenteral, rectal, ocular, intravenous or otic administration, or administration by inhalation.

Embodiment 89. A method of treating a condition or disease in a subject in need thereof, comprising administering a pharmaceutical composition of any one of Embodiments 84-87.

Embodiment 90. The method of Embodiment 89, wherein administering the pharmaceutical composition results in inhibiting mTORC₁ and/or mTORC_{2.}

Embodiment 91. The method of Embodiment 89 or 89, wherein administering the pharmaceutical composition further results in promoting immune cell differentiation

Embodiment 92. The method of any one of Embodiments 88-90, wherein administering the pharmaceutical composition results in suppression of proliferation of effector T-cells.

Embodiment 93. The method of any one of Embodiments 88-92, wherein administering the pharmaceutical composition results in differentiation of memory T-cells.

Embodiment 94. The method of any one of Embodiments 88-92, wherein administering the pharmaceutical composition results in differentiation of regulatory T-cells.

Embodiment 95. The method of any one of Embodiments 89-93, wherein administering the pharmaceutical composition results in cellular senescence.

Embodiment 96. The method of any one of Embodiments 88-94, wherein the condition or disease is a neurodegenerative disease.

Embodiment 97. The method of Embodiment 96, wherein the neurodegenerative disease is Alzheimer Disease, Parkinson Disease, Parkinson-like Disease, Huntington Disease, Lou Gehrig Disease, Multiple Sclerosis, autoimmune disorders, Pick Disease, diffuse Lewy body Disease, progressive supranuclear palsy (Steel-Richardson syndrome), multisystem degeneration (Shy-Drager syndrome), motor neuron diseases, amyotrophic lateral sclerosis, degenerative ataxias, cortical basal degeneration, ALS-Parkinson-Dementia complex of Guam, subacute sclerosing panencephalitis, synucleinopathies, primary progressive aphasia, striatonigral degeneration, Machado-Joseph disease/spinocerebellar ataxia type 3 and olivopontocerebellar degenerations, Gilles De La Tourette Disease, bulbar and pseudobulbar palsy, spinal and spinobulbar muscular atrophy (Kennedy Disease), primary lateral sclerosis, familial spastic paraplegia, Werdnig-Hoffmann Disease, Kugelberg-Welander Disease, Tay-Sach Disease, Sandhoff Disease, familial spastic disease, Wohlfart-Kugelberg-Welander Disease, spastic paraparesis, progressive multifocal leukoencephalopathy, prion diseases, including Creutzfeldt-Jakob Disease, Gerstmann-Straussler-Scheinker Disease, Kuru, and fatal familial insomnia.

Embodiment 98. The method of Embodiment 97, wherein the neurodegenerative disease is Alzheimer Disease.

Embodiment 99. The method of any one of Embodiments 88-94, wherein the condition or disease is a viral infection.

Embodiment 100. The method of Embodiment 99, wherein the viral infection is caused by a coronavirus.

Embodiment 101. The method of Embodiment 99, wherein the coronavirus is Alphacoronavirus, Betacoronavirus, a Gammacoronavirus, Deltacoronavirus, 229E coronavirus, NL63 coronavirus, 0C₄₃ coronavirus, HKU1 coronavirus, middle east respiratory syndrome related coronavirus (MERS-CoV), severe acute respiratory syndrome coronavirus (SARS-CoV), severe acute respiratory syndrome coronavirus 2 (SARS-CoV-2), a mutated form of any of these, or any combination thereof.

Embodiment 102. The method of Embodiment 100, wherein administering the pharmaceutical composition further comprises co-administration of a vaccine.

Embodiment 103. The method of Embodiment 102, wherein co-administration results in improved effectiveness of the vaccine.

Embodiment 104. The method of any one of Embodiments 98-102, wherein administering the pharmaceutical composition is effective to at least partially reduce a viral load of a coronavirus.

Embodiment 105. The method of any one of Embodiments 98-104, wherein the subject has or was previously diagnosed with a general symptom of a coronavirus.

Embodiment 106. The method of Embodiment 105, wherein the general symptom comprises a fever, a cough, shortness of breath, breathing difficulties, or any combination thereof.

Embodiment 107. A kit comprising a compound according to any one of Embodiments 1-49, a conjugate of any one of Embodiments 50-84, or a pharmaceutical composition of any one of Embodiments 85-88.

Embodiment 108. The kit of Embodiment 107, further comprising instructions for using the pharmaceutical composition.

Embodiment 109. The kit of Embodiment 107 or 108, further comprising a coronavirus vaccine.

Embodiment 110. A process for producing a biomolecule-drug-conjugate, comprising: (a) providing a compound of formula (I), (I-A) or (I-B) according to any one of Embodiments 1-49; (b) linking a linker L to the compound of formula (I), (I-A) or (I-B) to obtain a conjugate; and (c) linking the conjugate of step (b) to a biomolecule so as to obtain a biomolecule-drug-conjugate.

Embodiment 111. A process for producing a biomolecule-drug-conjugate, comprising conjugating a compound of formula (I), (I-A) or (I-B) according to any one of Embodiments 1-49 to a biomolecule.

Embodiment 112. The process of Embodiment 110 or 111, further comprising purifying the biomolecule-drug-conjugate.

Embodiment 113. The process of any one of Embodiments 110-112, wherein the biomolecule is an antibody.

Embodiment 114. The process of Embodiment 113, wherein the conjugating is site specific on one or more engineered cysteine and/or glutamine residues on the antibody.

Embodiment 115. A method of treating a condition or disease in a subject in need thereof, comprising administering the compound of any one of Embodiments 1-84 or the pharmaceutical composition of any one of Embodiments 85-88, thereby treating the condition or disease in the subject.

Embodiment 116. Use of the compound of any one of Embodiments 1-84 or the pharmaceutical composition of any one of Embodiments 85-88 for the treatment of a condition or disease in a subject in need thereof, the use comprising administering to the subject the compound or the pharmaceutical composition, thereby treating the condition or disease in the subject.

Embodiment 117. Use of the compound of any one of Embodiments 1-84 or the pharmaceutical composition of any one of Embodiments 85-88 for the manufacture of a medicament for the treatment of a condition or disease in a subject in need thereof, the use comprising administering to the subject an effective amount of the compound or the pharmaceutical composition, thereby treating the condition or disease in the subject.

Embodiment 118. The method of any one of Embodiments 115-118, wherein administering the compound or the pharmaceutical composition results in inhibiting mTORC₁ and/or mTORC2.

### EXAMPLE S

The following examples are provided to further illustrate some embodiments of the present disclosure, but are not intended to limit the scope of the disclosure; it may be understood by their exemplary nature that other procedures, methodologies, or techniques known to those skilled in the art may alternatively be used.

### Example 1

### Generation of the Bacterial Strains to Synthesize Compounds

The DNA fragments, named Left Arm, AT coding region (AT), Right Arm, and pUC₁₉ backbone, were amplified by PCR using the primers and templates listed in the proceeding Examples outlined below, respectively. The PCR products were separated by DNA electrophoresis, and the target fragments were purified. These four purified fragments were assembled using NEBuilder® HiFi DNA Assembly Master Mix (New England Biolabs, MA) to afford the intermediate plasmid. After validating the sequence of the insert in the intermediate plasmid, this plasmid was subjected to restriction digestion using the restriction enzyme(s) listed in each Example. The digestion products were separated by DNA electrophoresis, and the insert fragment was purified. The purified fragment was ligated with pKC₁₁₃₉ backbone, priorly linearized using the same restriction enzyme(s), using T4 DNA ligase (Thermo Fisher Scientific, MA) to afford the engineering plasmid.

The engineering plasmid was introduced into S. rapamycinicus ATCC 29253 by conjugation between E. coli and Streptomyces. The plasmid was transformed into E. coli S17-1 and plated on Luria-Bertani (LB) agar containing 100 mg/L apramycin (Teknova, Calif.). After incubating at 30° C. until the appearance of colonies, a single colony was inoculated into 2 mL LB media containing 25 mg/L apramycin in a 5 mL round-bottom tube for growing overnight at 30° C. 0.5 mL of the overnight culture was inoculated into 25 mL LB media containing 25 mg/L apramycin in a 250 mL Erlenmeyer flask for growing at 30° C. until OD600 reached 0.4. The culture was centrifuged at 4,000 g for 5 min. After removing the supernatant, the pellet cells were washed with 25 mL LB media three times. The washed cells were resuspended in 2.5 mL LB media and put on ice. S. rapamycinicus ATCC 29253 spores stock containing 1×10⁹ spores in 20% glycerol solution was centrifuged at 2,300 g for 5 min. After removing the supernatant, the spores were washed with Difco 2×YT medium (BD, NJ) three times. The spores were resuspended in 100 μL 2×YT media, and the suspension was treated by heat shock at 50° C. for 10 min in a water broth. After cooling down to room temperature, 100 μL spore suspension was mixed with 100 μL washed E. coli cells suspension gently. The mixture was spread on dry Mannitol Soy (MS) agar plate evenly, and the cultures were incubated at 29° C. for 18 hours. 1 mL aqueous solution containing 1.5 mg nalidixic acid sodium salt and 0.6 mg apramycin, pH=7, was added onto the grown cultures to overlay the whole plate evenly. After drying the plate surface in the biosafety cabinet, the culture was incubated at 29° C. until the conjugants appeared (5-7 days). The conjugants were streaked on 2CM plates (soluble starch 10 g, sodium chloride 1 g, peptone 2 g, dipotassium phosphate 1 g, magnesium sulfate heptahydrate 2 g, calcium carbonate 2 g, ammonium sulfate 2 g, trace element solution (iron(II) sulfate heptahydrate 1 g/L, magnesium chloride hexahydrate 1 g/L, zinc sulfate heptahydrate 1 g/L) 1 mL, difco agar 22 g, distilled water up to 1 L, pH=7.2) containing 50 mg/L nalidixic acid sodium salt and 25 mg/L apramycin. The streaked plate was first incubated at 30° C. for 24 h and then incubated at 37° C. until colonies appeared (3-5 days). These colonies were picked and streaked on new 2CM plates containing nalidixic acid sodium salt and apramycin, and the plates were incubated at 37° C. until colonies appeared (3 days). The resulting colonies were single crossover strains. The single crossover strains were grown on ISP Medium No. 3 (without antibiotics) at 30° C. for three generations. The resulting strains were streaked on ISP Medium No. 3, and the single colonies were screened by solid-state fermentation assay. The single colonies were grown on AMO agar (mixture 1: soybean meal 15.4 g, dipotassium phosphate 5 g, L-lysine hydrochloride 17.5 g, difco agar 20 g, distilled water up to 950 mL, pH=6.0. mixture 2: mannose 25.5 g, distilled water up to 50 mL. Mix these two mixtures after autoclaving at 121° C. for 20 min) for 3 days, respectively. The culture on the agar was stamped using a 1000-4, wide-bore pipette tip and transferred into a well of 96-well PCR plate. 100 μL methanol was added to the well, and the PCR plate was sealed with an adhesive foil. The plate was shaken at 37° C., 300 rpm for 30 min, then centrifuged at 4000 g for 10 min. 1μL supernatant was subjected to LC-MS analysis using an Agilent 1290/Ultivo Triple Quad LC/MS on a Kinetex column (Biphenyl 100Å, 1.7 μm, 50×2.1 mm, Phenomenex Inc., Calif.) heated to 40° C. by gradient elution of solvent A (H₂Od containing 0.1% formic acid) and solvent B (methanol containing 0.1% formic acid) at a flow rate of 0.75 mL/min as following program: T=0, 70% B; T=0.1 min, 70% B; T=1.1 min, 100% B; T=1.3 min, 100% B; T=1.31 min, 70% B; T=1.8 min, 70% B. UV absorption at 210 nm and 280 nm were recorded. The MS was run on negative mode. The precursor ions derived from newly produced compounds were subjected to LC-MS/MS analysis, in which the precursor ion ([M+HCOO]⁻) was fragmented under a voltage at 135 V.

The strains that didn't produce rapamycin but produced Compound were spread on ISP Medium No. 3 supplemented with 50 mM calcium chloride to grow at 29° C. until sporulation (14 days). The spores were collected in 20% glycerol solution and filtered through a 100 μm cell strainer. The filtrate was stored in a −80° C. ultra-low freezer.

### Example 2

### Method to Produce Compounds

20 μL spore suspension was used to inoculate 50 mL seed media (yeast extract 5 g, malt extract 4 g, maltose 5 g, L-lysine hydrochloride 5 g, distilled water up to 1 L, pH =6) in a 250 mL Erlenmeyer flask with milk filter cap. After growing at 28° C. for 3 days, 5 mL of the seed culture was inoculated into 1 L fermentation media (mixture 1: soybean meal 15.4 g, dipotassium phosphate 5 g, L-lysine hydrochloride 17.5 g, distilled water up to 950 mL, pH=6.0. mixture 2: mannose 25.5 g, distilled water up to 50 mL. Mix these two mixtures after autoclaving at 121° C. for 20 min) in a 2.8-L baffled Fernbach flask with a milk filter cap. 4 L fermentation was conducted at 28° C., 220 rpm for 5 days, then the fermentation broth was centrifuged at 4000 g for 20 min at room temperature, and the cell pellet was collected and combined. After transferring the cells into a 1-L glass bottle, 500 mL of acetone was added to the cells. The mixture was shaken vigorously and allowed to settle for 2 hours, then filtered through sintered glass funnel, with sand as the filter aid. The filtrate was concentrated under reduced pressure to remove acetone. The left liquid was extracted by ethyl acetate three times, and the ethyl acetate phase was combined and washed with saturated sodium bicarbonate solution and saturated brine. The organic phase was filtered through anhydrous sodium sulfate and concentrated under reduced pressure. The residue was subjected to purification steps to afford the final compound, as follows:

Flash Step: the residue was dissolved in 5 mL dichloromethane and separated on an Agela Flash Column Silica-CS (40 g) column using a BUCHI Pure C-805 Flash system by gradient elution of solvent A (dichloromethane) and solvent B (methanol) at a flow rate of 50 mL/min as the following program: T=0, 0% B; T=15 min, 6% B; T=17 min, 6% B. All elute was collected in 25-mL fractions, and the fractions were subjected to LC-MS analysis. The fractions containing the target compound were combined and concentrated under reduced pressure.

Prep Step A: The residue from Flash Step was suspended in 45% acetonitrile aqueous solution and injected onto a Kinetex column (5 μm, F5 100 Å, 150×21.2 mm, Phenomenex Inc., CA) for separation using a BUCHI pure C-830 Prep system by isocratic elution of 45% acetonitrile with 55% water at a flow rate of 25 mL/min. All elute was collected in 25-mL fractions, and the fractions were subjected to LC-MS analysis. Those fractions containing the compound were combined and concentrated under reduced pressure.

Prep Step B: the residue from Prep Step A was dissolved in 60% methanol aqueous solution and subjected to preparative HPLC purification using the same system by gradient elution of solvent A (water) and solvent B (methanol) at a flow rate of 25 mL/min as the following program: T=0, 60% B; T=1 min, 60% B; T=21 min, 90% B; T=21 min, 100% B; T=24 min, 100% B; T=24 min, 60% B; T=27 min, 60% B. All elute was collected in 25-mL fractions, and the fractions were subjected to LC-MS analysis. Those fractions containing the pure compound were combined and concentrated under reduced pressure.

### Example 3

### Anti-Yeast Activity Assay

A single colony of Saccharomyces cerevisiae BY4742 was inoculated into 2 mL YPD media in a 5 mL falcon tube to grow overnight at 30° C. The overnight culture was inoculated into 5 mL YPD media in a glass tube at OD600=0.1, and the new culture was grown at 30° C. until OD600 reached 0.5. The culture was then diluted to OD600=0.01 and aliquoted into 96-well plates at 100 μL per well. The compound was dissolved in ethanol at 75 μM. Three-fold serial dilutions were conducted for the Compound 10 times using ethanol, respectively. 4 μL of each concentration compound solution was added and mixed with 384 μL YPD media. 100 μL of the YPD media solutions were mixed with the Saccharomyces cerevisiae cultures in 96-well plates by pipette, respectively. The plates were incubated at 30° C. for 16 hours without shaking. The lowest Compound concentrations in wells that have no obvious culture growth were defined as the minimum inhibitory concentrations (MICs). The assays were conducted in triplicate.

### Example 4

### Synthesis of Compound 9

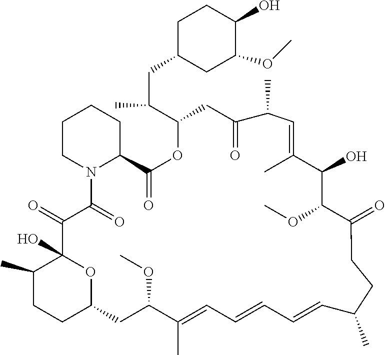 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@](C)(O)[C@@H](OC)C(=O)CC[C@H](C)/C=C/C=C/C=C/1C]

The synthesis outlined in Example 1 was conducted to generate the strain to produce (3S,6R,7E,9R,10R,14S,15E,17E,19E,21S,23S,26R,27R,34aS)-9,27-dihydroxy-3-((R)-1-((1S,3R,4R)-4-hydroxy-3-methoxycyclohexyl)propan-2-yl)-10,21-dimethoxy-6,8,14,20,26-pentamethyl-9,10,12,13,14,21,22,23,24,25,26,27,32,33,34,34a-hexadecahydro-3H-23,27-epoxypyrido[2,1-c] [1] oxa[4]azacyclohentriacontine-1,5,11,28,29(4H,6H,31H)-pentaone (Compound 9).

TABLE 4
Templates and primers used for Compound 9:

| Target | | Fragment | | |
|---|---|---|---|---|
| fragment | Template | Length | Primers | |
| Left Arm | S. rapamycinicus | 2 kb | pAP001_L_F | TACCCGGGGATCCTTCTAGAA |
| | genomic DNA | | | GACCGTATTCCGGCCCAAG |
| | | | pAP001_L_R | ATGACGTGGGCGTTCGTACCG |
| | | | | CTGACTCCGAAGGACGACA |
| Right Arm | S. rapamycinicus | 2 kb | pAP001_R_F | CGGCCACCCGGGTGCTGGACC |
| | genomic DNA | | | TTCCGACCTACGCCTTCCAAC |
| | | | | A |
| | | | pAP001_R_R | CCTGCAGGTCGACTTCTAGAT |
| | | | | CGCCACGATGCGATGCGCT |
| pUC19 | pUC19 | 2.6 kb | pAP001_19_F | TCGCATCGTGGCGATCTAGAA |
| vector | | | | GTCGACCTGCAGGCATGCA |
| | | | pAP001_19_R | CCGGAATACGGTCTTCTAGAA |
| | | | | GGATCCCCGGGTACCGAGC |
| AT | pAP00116 | 1.2 kb | pAP001_AT8_F | TGTCGTCCTTCGGAGTCAGCG |
| | | | | GTACGAACGCCCACGTCATCC |
| | | | pAP001_AT8_R | TGGAAGGCGTAGGTCGGAAGG |
| | | | | TCCAGCACCCGGGTGGCCG |

pAP00116 contains the following synthetic DNA sequence encoding the AT domain in the 8^{th} module of rapamycin PKS:

GGTACGAACGCCCACGTCATCCTGGAATCGGCACCGCCGACCCAGCCCG
CCGACAACGCGGTGATCGAGCGGGCGCCGGAATGGCTGCCCATGGTGAT
CAGTGCACGTACGCAGTCCGCCCTGACCGAACACGAAGGCCGGTTAAGA
GCGTACCTGGCTGCCAGCCCGGGCGTGGATATGCGGGCCGTTGCATCGA
CGTTGGCCATGACACGGTCGGTGTTCGAGCACCGCGCTGTTCTGTTGGG
TGACGACACCGTGACCGGCACCGCGGCGACCGACCCGCGTGTCGTCTTC
GTCTTCCCCGGTCAGGGTTCGCAGCGGGCTGGGATGGGCGAAGAGTTGG
CAGCAGCGTTCCCGGTGTTCGCTCGGATCCATCAGCAGGTGTGGGACCT
GCTGGATGTGCCGGATCTGGAGGTGAACGAGACCGGGTACGCGCAGCCG
GCGCTGTTCGCCTTGCAGGTCGCTCTGTTCGGGTTGTTGGAGTCCTGGG
GTGTTCGACCGGACGCGGTGGTCGGCCACAGTGTCGGAGAACTGGCGGC
CGGGTATGTCTCGGGTCTGTGGTCGCTGGAGGACGCCTGCACGCTGGTG
AGCGCAAGAGCACGGCTGATGCAGGCATTACCTGCTGGTGGTGTGATGG
TCGCGGTCCCGGTCTCCGAAGATGAGGCGAGGGCAGTTTTAGGTGAGGG
TGTCGAGATCGCCGCCGTGAACGGCCCCAGCAGCGTGGTGCTGTCCGGC
GACGAGGCTGCTGTCCTGCAGGCCGCTGAGGGGCTGGGGAAGTGGACGA
GATTAGCAACCAGCCACGCCTTCCACTCGGCCCGGATGGAGCCGATGCT
GGAGGAGTTCAGGACCGTCGCCGAGGGCCTGACGTACCGGACCCCGCAG
GTGTCGATGGCCGCGGGTGATCAGGTCACCACCACGGAGTACTGGGTGC
GGCAGGTTCGTGACACGGTCCGGTTCGGCGAGCAGGTGGCCTCGTACGA
GGATGCGGTGTTCGTGGAGCTGGGTGCGGATCGCTCGTTGGCGAGACTG
GTCGACGGCGTTGCGATGTTGCACGGCGATCACGAGGCCCAGGCGGCTG
TGTCGGCGTTGGCTCACCTGTACGTCAACGGTGTGACGGTGGACTGGCC
GGCATTGCTGGGAGATGCCCCGGCCACCCGGGTGCTGGAC

Intermediate plasmid name: pAP00007

Restriction enzymes to construct the engineering plasmid: EcoRI and HindIII

Engineering plasmid name: pAP00008

Strain name: AS 71

Compound 9 was detected by LC-MS and LC-MS/MS in the solid-state fermentation assay: HPLC retention time: 0.936 min; LC-MS m/z: [M +HCOO]⁻ calc. 944.5 for C₅₁H₇₈NO₁₅, found 944.3. LC-MS/MS m/z: Frag. A calc. 321.2 for C₁₉H₂₉O₄, found 321.1; Frag. B calc. 576.3 for C₃₁H₄₆NO₉, found 576.0.

### Example 5

### Synthesis of Compound 10

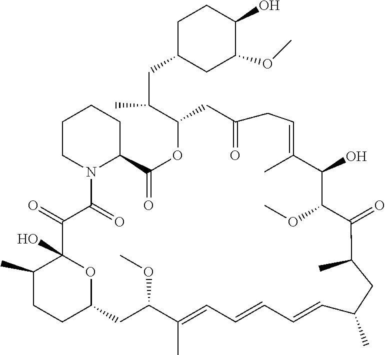 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)C/C=C(\C)[C@@](C)(O)[C@@H](OC)C(=O)[C@H](C)C[C@H](C)/C=C/C=C/C=C/1C]

The synthesis outlined in Example 1 was conducted to generate the strain to produce (3S,7E,9R,10R,12R,14S,15E,17E,19E,21S,23S,26R,27R,34aS)-9,27-dihydroxy-3-((R)-1-((1S,3R,4R)-4-hydroxy-3-methoxycyclohexyl)propan-2-yl)-10,21-dimethoxy-8,12,14,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 10).

TABLE 5
Templates and primers used for Compound 10:

| Target | | Fragment | | |
|---|---|---|---|---|
| fragment | Template | Length | Primers | |
| Left Arm | S. rapamycinicus | 2 kb | pAP00018_L_F | TACCCGGGGATCCTTCTAGATGGATGA |
| | genomic DNA | | | CGGTGTTGTGGAG |
| | | | pAP00009_AT3_L_R | ATGACGTGGGCGTTCGTACCGCTGACT |
| | | | | CCGAAGGACGACA |
| Right Arm | S. rapamycinicus | 2 kb | pAP00009_AT3_R_F | CGGCCACCCGGGTGCTGGACCTTCCGA |
| | genomic DNA | | | CCTACGCCTTCCA |
| | | | pAP00009_AT3_R_R | CCTGCAGGTCGACTTCTAGACGGTAGT |
| | | | | GTTCAGGAACGCT |
| pUC19 | pUC19 | 2.6 kb | pAP00009_19_F | CCTGAACACTACCGTCTAGAAGTCGAC |
| vector | | | | CTGCAGGCATGCA |
| | | | pAP00018_19_R | AACACCGTCATCCATCTAGAAGGATCC |
| | | | | CCGGGTACCGAGC |
| AT | pAP00116 | 1.2 kb | pAP00009_AT8_F | TGTCGTCCTTCGGAGTCAGCGGTACGA |
| | | | | ACGCCCACGTCATCC |
| | | | pAP00009_AT8_R | TGGAAGGCGTAGGTCGGAAGGTCCAGC |
| | | | | ACCCGGGTGGCCG |

Intermediate plasmid name: pAP00018

Restriction enzyme to construct the engineering plasmid: XbaI

Engineering plasmid name: pAP00010

Strain name: AS_73

Compound 10 was detected by LC-MS and LC-MS/MS in the solid-state fermentation assay: HPLC retention time: 0.936 min; LC-MS m/z: [M+HCOO]⁻ calc. 944.5 for C₅₁H₇₈NO₁₅, found 944.6. LC-MS/MS m/z: Frag. A calc. 307.1 for C₁₈H₂₇O₄, found 307.2; Frag. B calc. 590.3 for C₃₂H₄₈NO₉, found 589.9.

The experimental procedure outlined in Example 2 was conducted for the synthesis of Compound 10, the following fractions were collected. Retention time=11.1-13.1 min in Flash Step. Retention time=20.2-24.3 min in Prep Step A. Prep Step B was not conducted. 12.8 mg of Compound 10 was prepared as amorphous yellow solid.

The experimental procedure outlined in Example 3 was conducted to assay the MIC value: MIC against Saccharomyces cerevisiae BY4742: lower than 55.6 nM.

### Example 6

### Synthesis of Compound 11

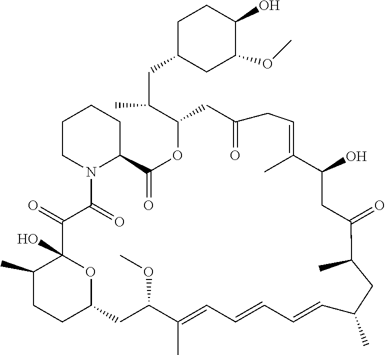 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)C/C=C(\C)[C@@](C)(O)CC(=O)[C@H](C)C[C@H](C)/C=C/C=C/C=C/1C]

LC-MS and LC-MS/MS analysis indicated the strain, AS_73, generated in Example 13 (Synthesis of Compound 10) also produced (3S,7E,9S,12R,14S,15E,17E,19E,21S,23S,26R,27R,34aS)-9,27-dihydroxy-3-((R)-1-((1S,3R,4R)-4-hydroxy-3-methoxycyclohexyl)propan-2-yl)-21-methoxy-8,12,14,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 11) in the solid-state fermentation assay: HPLC retention time: 0.953 min; LC-MS m/z: [M+HCOO]⁻ calc. 914.5 for C₅₀H₇₆NO₁₄, found 914.7. LC-MS/MS m/z: Frag. A calc. 307.1 for C₁₈H₂₇O₄, found 307.3; Frag. B calc. 560.3 for C₃₁H₄₆NO₈, found 560.6.

### Example 7

### Synthesis of Compound 12

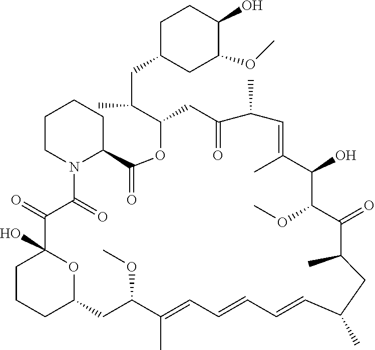 [SMILES: CO[C@H]1C[C@@H]2CCC[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

The synthesis outlined in Example 1 was conducted to generate the strain to produce (3S,6R,7E,9R,10R,12R,14S,15E,17E,19E,21S,23S,27R,34aS)-9,27-dihydroxy-3-((R)-1-((1S,3R,4R)-4-hydroxy-3-methoxycyclohexyl)propan-2-yl)-10,21-dimethoxy-6,8,12,14,20-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 12).

TABLE 6
Templates and primers used of Compound 12:

| Target | | Fragment | | |
|---|---|---|---|---|
| fragment | Template | Length | Primers | |
| Left Arm | S. rapamycinicus | 2 kb | pAP00015_L_F | TACCCGGGGATCCTTCTAGATGCGGCC |
| | genomic DNA | | | CAAGGCCGACGGC |
| | | | pAP00015_L_R | ATGACGTGGGCGTTCGTACCGCTGACT |
| | | | | CCGAAGGACGACACGCC |
| Right Arm | S. rapamycinicus | 2 kb | pAP00015_R_F | CGGCCACCCGGGTGCTGGACCTTCCGA |
| | genomic DNA | | | CCTACGCCTTCCA |
| | | | pAP00015_R_R | CCTGCAGGTCGACTTCTAGACTCAGTG |
| | | | | TGCGGGAGTCGGC |
| pUC19 | pUC19 | 2.6 kb | pAP00015 19_F | TCCCGCACACTGAGTCTAGAAGTCGAC |
| vector | | | | CTGCAGGCATGCA |
| | | | pAP00015_19 R | GGCCTTGGGCCGCATCTAGAAGGATCC |
| | | | | CCGGGTACCGAGC |
| AT | pAP00116 | 1.2 kb | pAP00015_AT8_F | TGTCGTCCTTCGGAGTCAGCGGTACGA |
| | | | | ACGCCCACGTCATCC |
| | | | pAP00015_AT8_R | TGGAAGGCGTAGGTCGGAAGGTCCAGC |
| | | | | ACCCGGGTGGCCG |

Intermediate plasmid name: pAP00015

Restriction enzyme to construct the engineering plasmid: Xbal

Engineering plasmid name: pAP00016

Strain name: AS_81

Compound 12 was detected by LC-MS and LC-MS/MS in the solid-state fermentation assay: HPLC retention time: 0.901 min; LC-MS m/z: [M+HCO]⁻ calc. 944.5 for C₅₁H₇₈NO₁₅, found 944.6. LC-MS/MS m/z: Frag. A calc. 321.2 for C₁₉H₂₉O₄, found 321.2; Frag. B calc. 576.3 for C₃₁H₄₆NO₉, found 576.0.

The experimental procedure outlined in Example 2 was conducted for the synthesis of Compound 12, the following fractions were collected: Retention time=10.9-12.5 min in Flash Step. Retention time=13.2-16.2 min in Prep Step A. Retention time=16.2-17.2 min in Prep Step B. Retention time=16.8-18.2 min in an additional preparative HPLC purification step using the same method described in Prep Step B except the gradient program was used: T=0, 60% B; T=1 min, 60% B; T=21 min, 85% B; T=21 min, 100% B; T=24 min, 100% B; T=24, 60% B; T=27 min, 60% B. The fractions were collected in the peak collection mode at 5 mL/fraction. 10 mg of Compound 12 was prepared as amorphous yellow solid.

The experimental procedure outlined in Example 3 was conducted to assay the MIC value: MIC against Saccharomyces cerevisiae BY4742: lower than 55.6 nM.

### Example 8

### Synthesis of Compound 13

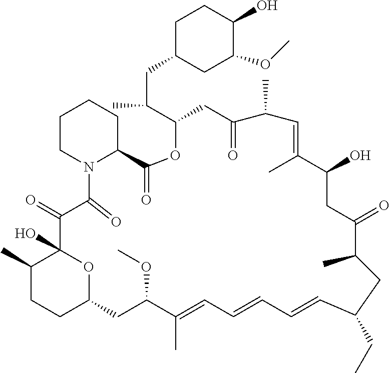 [SMILES: CC[C@@H]1/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)CC(=O)[C@H](C)C1]

The synthesis outlined in Example 1 was conducted to generate the strain to produce (3S,6R,7E,9S,12R,14S,15E,17E,19E,21S,23S,26R,27R,34aS)-14-ethyl-9,27-dihydroxy-3-((R)-1-((1S,3R,4R)-4-hydroxy-3-methoxycyclohexyl)propan-2-yl)-21-methoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 13).

TABLE 7
Templates and primers used for Compound 13:

| Target | | Fragment | | |
|---|---|---|---|---|
| fragment | Template | Length | Primers | |
| Left Arm | S. rapamycinicus | 2 kb | pAP00017_L_F | TACCCGGGGATCCTTCTAGAACGACGG |
| | genomic DNA | | | CGTCTTGGAGACC |
| | | | pAP00017_L_R | AGGACGTGGGCGTTGGTGCCACTGACC |
| | | | | CCGAAGGCCGACA |
| Right Arm | S. rapamycinicus | 2 kb | pAP00017_R_F | CGACGGCGACGGCACACGACCTTCCGA |
| | genomic DNA | | | CGTACGCGTTCCA |
| | | | pAP00017_R_R | CCTGCAGGTCGACTTCTAGAATTTCCC |
| | | | | GGAAGCCAGTGGT |
| pUC19 | pUC19 | 2.6 kb | pAP00017_19_F | GGCTTCCGGGAAATTCTAGAAGTCGAC |
| vector | | | | CTGCAGGCATGCA |
| | | | pAP00017_19_R | CAAGACGCCGTCGTTCTAGAAGGATCC |
| | | | | CCGGGTACCGAGC |
| AT | pAP00021 | 1.3 kb | pAP00017_520AT4_F | TGTCGGCCTTCGGGGTCAGTGGCACCA |
| | | | | ACGCCCACGTCCT |
| | | | pAP00017_520AT4_R | TGGAACGCGTACGTCGGAAGGTCGTGT |
| | | | | GCCGTCGCCGTCG |

pAP00021 contains the following synthetic DNA sequence encoding the AT domain in the 4^{th} module of FK520 PKS:

GGCACCAACGCCCACGTCCTGCTGGAAGCCCACCCGGCCGGGGAGCCGC
CGGCCGAGGAGCCGTCGGCCTCGAAGCCCGGTGAGCCGCTGATCGCCAC
GCCGCTCACACCACTGCCCGTCTCGGCGCGGACCGCCACGGCCCTCGAC
GGCCAGGTCCGCCGACTCCGCGAGCACCTCGCCGCCCGTCCCGGCCACG
ACCCGCGCGCCATCGCCGCGGGCCTGCTCGCCAGGCGTACGACGTTCCC
GCACCGCGCCGTGCTGCTCGACGACGACGTCGTCACCGGCACGGCGCTC
ACCGAGCCGCGCACCGTCTTCGTCTTCCCCGGACAAGGACCGCAGTGGC
GCGGCATGGGCGTCGAACTGATGGCGGCCTCCCCGGTGTTCGCCGCCAG
GATGCGCCAATGCGCCGACGCGCTGATCCCGCACACGGGCTGGGACCCC
ATCGCCATGCTCGACGACCCGGAGGTGACCCGCCGCGTCGACGTCGTGC
ACCCCGTCTGCTGGGCCGTCATGGTGTCGCTGGCCGCCGTGTGGGAGGC
CGCGGGCGTACGCCCGGACGCCGTCATCGGACACTCCCAGGGCGAGATC
GCCGCGGCCTGTGTCGCCGGAGCGCTCACCCTGGAGGACGGTGCCCGCC
TCGTCGCGCTGCGCAGCGCCCTGCTCCTGCGCGAACTCGCCGGACGCGG
CGCGATGGGCTCGGTCGCGCTCCCGGCCGCCGACGTCGAGGCGGATGCC
GCCCGGATCGACGGCGTCTGGGTCGCGGGCCGCAACGGCGCCACCACCA
CGACCGTCGCCGGGCGCCCGGACGCCGTCGAAACGCTGATCGCCGACTA
CGAGGCCCGCGGCGTCTGGGTGCGCCGCATCGCCGTCGACTGCCCGACC
CACACCCCGTTCGTCGACCCGCTGTACGACGAACTCCAGCGGATCGTCG
CGGACACCACCTCGCGCACGCCCGAGATCCCGTGGTTCTCCACCGCCGA
CGAACGCTGGATCGACGCGCCGCTCGACGACGAGTACTGGTTCCGCAAC
ATGCGCCACCCCGTAGGCTTCGCCACGGCCGTGACCGCTGCCCGCGAGC
CGGGTGACACCGTGTTCGTCGAGGTCAGCGCGCACCCCGTGCTGCTGCC
CGCGATCGACGGCGCGACCGTCGCCACGCTCCGCCGCGGCGGGGGAGTC
CACCGGCTGCTCACCGCGCTGGCCGAGGCGCACACAACCGGCGTGCCCG
TCGACTGGGCGGCGGTCGTCCCCGCGACGGCGACGGCACACGAC

Intermediate plasmid name: pAP00021

Restriction enzyme to construct the engineering plasmid: XbaI

Engineering plasmid name: pAP00027

Strain name: AS_84

Compound 13 was detected by LC-MS and LC-MS/MS in the solid-state fermentation assay: HPLC retention time: 0.997 min; LC-MS m/z: [M+HCOO]⁻ calc. 942.5 for C₅₂H₈₀NO₁₄, found 942.5. LC-MS/MS m/z: Frag. A calc. 321.2 for C₁₉H₂₉O₄, found 321.4; Frag. B calc. 574.3 for C₃₂H₄₈NO₈, found 573.9.

### Example 9

### Synthesis of Compound 14

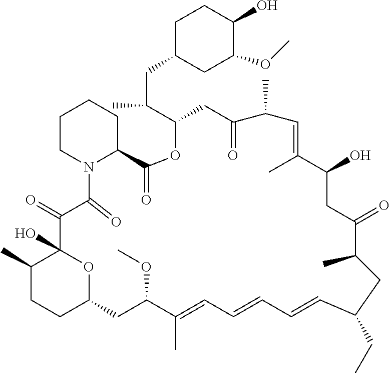 [SMILES: CC[C@@H]1/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)CC(=O)[C@H](C)C1]

The synthesis outlined in Example 1 was conducted to generate the strain to produce (3S,6R,7E,9S,10R,12R,14S,15E,17E,19E,21S,23S,26R,27R,34aS)-9,27-dihydroxy-3-((R)-1-((1S,3R,4R)-4-hydroxy-3-methoxycyclohexyl)propan-2-yl)-21-methoxy-6,8,10,12,14,20,26-heptamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 14).

TABLE 8
Templates and primers used for Compound 14

| Target | | Fragment | | |
|---|---|---|---|---|
| fragment | Template | Length | Primers | |
| Left Arm | S. rapamycinicus | 2 kb | pAP00036_L_F | TACCCGGGGATCCTTCTAGAGGTGGCC |
| | genomic DNA | | | TGCGGGCCATCTC |
| | | | pAP00036_L_R | ATGACGTGGGCGTTGGTGCCACTGATC |
| | | | | CCGAAGGACGACACACC |
| Right Arm | S. rapamycinicus | 2 kb | pAP00036_R_F | CCACCACCCGCGTCCTGGACCTTCCGA |
| | genomic DNA | | | CATACGCCTTCCAGC |
| | | | pAP00036_R_R | CCTGCAGGTCGACTTCTAGAAATGCCG |
| | | | | GGACTTCCGCGTC |
| pUC19 | pUC19 | 2.6 kb | pAP00036_19_F | GAAGTCCCGGCATTTCTAGAAGTCGAC |
| vector | | | | CTGCAGGCATGCA |
| | | | pAP00036_19_R | GCCCGCAGGCCACCTCTAGAAGGATCC |
| | | | | CCGGGTACCGAGC |
| AT | pAP00120 | 1.2 kb | pAP00036_AT13_F | TGTCGTCCTTCGGGATCAGTGGCACCA |
| | | | | ACGCCCACGTCAT |
| | | | pAP00036_AT13_R | TGGAAGGCGTATGTCGGAAGGTCCAGG |
| | | | | ACGCGGGTGGTGG |

pAP00120 contains the following synthetic DNA sequence encoding the AT domain in the 13^{th} module of rapamycin PKS:

GGCACCAACGCCCACGTCATCCTGGAGTCCGCCCCGCCCGCCCAGCCGG
CCGAGGAGGCCCAGCCGGTCGAGACGCCCGTCGTCGCCTCCGACGTCCT
GCCGCTGGTCATCTCCGCCAAGACCCAGCCCGCCCTGACCGAGCACGAG
GACCGCCTGCGGGCCTACCTGGCCGCCTCCCCCGGCGCCGACATCCGGG
CCGTCGCCTCCACCCTGGCCGTCACCCGCTCCGTCTTCGAGCACCGGGC
CGTCCTGCTGGGCGACGACACCGTCACCGGCACCGCCGTCACCGACCCG
CGCATCGTCTTCGTCTTCCCCGGCCAGGGCTGGCAGTGGCTGGGCATGG
GCTCCGCCCTGCGCGACTCCTCCGTCGTCTTCGCCGAGCGGATGGCCGA
GTGCGCCGCCGCCCTGCGCGAGTTCGTCGACTGGGACCTGTTCACCGTC
CTGGACGACCCCGCCGTCGTCGACCGGGTCGACGTCGTCCAGCCCGCCT
CCTGGGCCATGATGGTGTCCCTGGCCGCCGTCTGGCAGGCCGCCGGCGT
CCGCCCGGACGCCGTCATCGGCCACTCCCAGGGCGAGATCGCCGCCGCC
TGCGTCGCCGGCGCCGTCTCCCTGCGCGACGCCGCCCGGATCGTCACCC
TGCGCTCCCAGGCCATCGCCCGCGGCCTGGCCGGCCGGGGCGCCATGGC
CTCCGTCGCCCTGCCGGCCCAGGACGTCGAGCTGGTCGACGGCGCCTGG
ATCGCCGCCCACAACGGCCCGGCCTCCACCGTCATCGCCGGCACCCCCG
AGGCCGTCGACCACGTCCTGACCGCCCACGAGGCCCAGGGCGTCCGGGT
CCGCCGGATCACCGTCGACTACGCCTCCCACACCCCGCACGTCGAGCTG
ATCCGCGACGAGCTGCTGGACATCACCTCCGACTCCTCCTCCCAGACCC
CGCTGGTCCCCTGGCTGTCCACCGTCGACGGCACCTGGGTCGACTCCCC
CCTGGACGGCGAGTACTGGTACCGCAACCTGCGGGAGCCGGTCGGCTTC
CACCCCGCCGTCTCCCAGCTGCAGGCCCAGGGCGACACCGTCTTCGTCG
AGGTGTCCGCCTCCCCGGTCCTGCTGCAGGCCATGGACGACGACGTCGT
CACCGTCGCCACCCTGCGCCGGGACGACGGCGACGCCACCCGGATGCTG
ACCGCCCTGGCCCAGGCCTACGTCCACGGCGTCACCGTCGACTGGCCCG
CCATCCTGGGCACCACCACCACCCGCGTCCTGGAC

Intermediate plasmid name: pAP00036

Restriction enzyme to construct the engineering plasmid: XbaI

Engineering plasmid name: pAP00045

Strain name: AS_109

### Example 10

### Synthesis of Compound 15

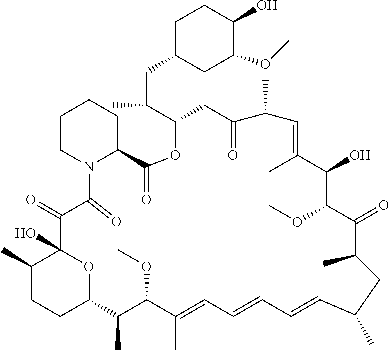 [SMILES: CO[C@@H]1/C(C)=C/C=C/C=C/[C@@H](C)C[C@@H](C)C(=O)[C@H](OC)[C@H](O)/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)[C@@H]1C]

The synthesis outlined in Example 1 was conducted to generate the strain to produce (3S,6R,7E,9R,10R,12R,14S,15E,17E,19E,21S,22S,23S,26R,27R,34aS)-9,27-dihydroxy-3-((R)-1-((1S,3R,4R)-4-hydroxy-3-methoxycyclohexyl)propan-2-yl)-10,21-dimethoxy-6,8,12,14,20,22,26-heptamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 15).

TABLE 9
Templates and primers used for Compound 15:

| Target | | Fragment | | |
|---|---|---|---|---|
| fragment | Template | Length | Primers | |
| Left Arm | S. rapamycinicus | 2 kb | pAP00039_L_F | TACCCGGGGATCCTTCTAGAGGATGCG |
| | genomic DNA | | | ACAGCCGCAGCGC |
| | | | pAP00039_L_R | ATGACGTGGGCGTTGGTGCCACTGACT |
| | | | | CCAAAGGAGGACACGCCTG |
| Right Arm | S. rapamycinicus | 2.5 kb | pAP00039_R_F | CCACCACCCGCGTCCTGGACCTTCCGA |
| | genomic DNA | | | CGTACGCCTTCCAGC |
| | | | pAP00039_R_R | CCTGCAGGTCGACTTCTAGACACGGTC |
| | | | | GGTGGGGAAACCG |
| pUC19 | pUC19 | 2.6 kb | pAP00039_19_F | CCCCACCGACCGTGTCTAGAAGTCGAC |
| vector | | | | CTGCAGGCATGCA |
| | | | pAP00039_19_R | CGGCTGTCGCATCCTCTAGAAGGATCC |
| | | | | CCGGGTACCGAGC |
| AT | pAP00120 | 1.2 kb | pAP00039_AT13_F | TGTCCTCCTTTGGAGTCAGTGGCACCA |
| | | | | ACGCCCACGTCAT |
| | | | pAP00039_AT13_R | TGGAAGGCGTACGTCGGAAGGTCCAGG |
| | | | | ACGCGGGTGGTGG |

Intermediate plasmid name: pAP00039

Restriction enzyme to construct the engineering plasmid: Xbal

Engineering plasmid name: pAP00048

Strain name: AS)117

Compound 15 was detected by LC-MS and LC-MS/MS in the solid-state fermentation assay: HPLC retention time: 0.979 min; LC-MS m/z: [M+HCOO]⁻ calc. 972.6 for C₅₃H₈₂NO₁₅, found 972.6. LC-MS/MS m/z: Frag. A calc. 321.2 for C₁₉H₂₉O₄, found 321.1; Frag. B calc. 604.3 for C₃₃H₅₀NO₉, found 604.5.

The experimental procedure outlined in Example 2 was conducted for the synthesis of Compound 15, the following fractions were collected. Retention time=11.2-12.9 min in Flash Step. Retention time=21.5 — 28.7 min in Prep Step A. Retention time=18.5-20 min in Prep Step B. Retention time=21.3-22.3 min in an additional preparative HPLC purification step using the same method described in Prep Step B except the gradient program was used: T=0, 60% B; T=1 min, 60% B; T=15.07 min, 80% B; T=35.07 min, 80% B; T=35.07 min, 100% B; T=38.07, 100% B; T=38.07 min, 60% B; T=41 min, 60% B. The fractions were collected in the peak collection mode at 5 mL/fraction. 2.6 mg of Compound 15 was prepared as amorphous white solid. The stereocenter at 22-C on Compound 15 was proposed as “S” based on bioinformatic analysis.

The experimental procedure outlined in Example 3 was conducted to assay the MIC value: MIC against Saccharomyces cerevisiae BY4742: lower than 55.6 nM.

### Example 11

### Synthesis of Compound 16

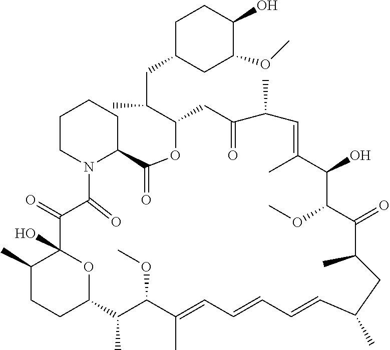 [SMILES: CO[C@@H]1/C(C)=C/C=C/C=C/[C@@H](C)C[C@@H](C)C(=O)[C@H](OC)[C@H](O)/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)[C@H]1C]

The stereocenter at 22-C on Compound 15 was proposed as “S” based on bioinformatic analysis. This analysis cannot exclude the compound has a “S” stereocenter at this position as (3S,6R,7E,9R,10R,12R,14S,15E,17E,19E,21S,22R,23S,26R,27R,34aS)-9,27-dihydroxy-34(R)-1-((1S,3R,4R)-4-hydroxy-3-methoxycyclohexyl)propan-2-yl)-10,21-dimethoxy-6,8,12,14,20,22,26-heptamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 16).

### Example 12

### Synthesis of Compound 17

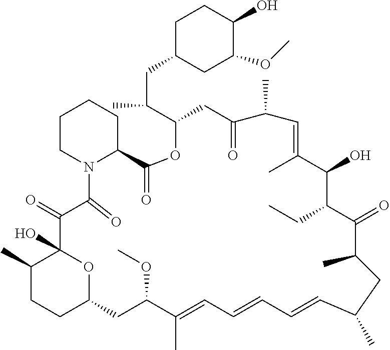 [SMILES: CC[C@H]1C(=O)[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@H]1O]

The synthesis outlined in Example 1 was conducted to generate the strain to produce (3S,6R,7E,9S,10R,12R,14S,15E,17E,19E,21S,23S,26R,27R,34aS)-10-ethyl-9,27-dihydroxy-3-((R)-1-((1S,3R,4R)-4-hydroxy-3-methoxycyclohexyl)propan-2-yl)-21-methoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 17).

TABLE 10
Templates and primers used for Compound 17:

| Target | | Fragment | | |
|---|---|---|---|---|
| fragment | Template | Length | Primers | |
| Left Arm | S. rapamycinicus | 2 kb | AT5_L_19_F | CTCGGTACCCGGGGATCCTTCTAGAGG |
| | genomic DNA | | | TGGCCTGCGGGCCATCTC |
| | | | AT5_L_EM_R | CCAGCAGGACGTGGGCGTTGGTGCCAC |
| | | | | TGATCCCGAAGGACGACACACCTGC |
| Right Arm | S. rapamycinicus | 2 kb | AT5_R_EM_F | GTCCCCGCGACGGCGACGGCACACGAC |
| | genomic DNA | | | CTTCCGACATACGCCTTCCAGCACCAG |
| | | | AT5_R_19_R | GCATGCCTGCAGGTCGACTTCTAGAAA |
| | | | | TGCCGGGACTTCCGCGTCC |
| pUC19 | pUC19 | 2 kb | pUC19_Xba_I_L_F | TCTAGAAGTCGACCTGCAGGCATGCA |
| vector | | | | |
| | | | pUCI9_Xba_I_L_R | TCTAGAAGGATCCCCGGGTACCGAGC |
| AT | pAP00021 | 1.3 kb | FK520_AT4_F | GGCACCAACGCCCACGTCCTG |
| | | | FK520_AT4_R | GTCGTGTGCCGTCGCCGTCG |

Intermediate plasmid name: pAP00041

Restriction enzyme to construct the engineering plasmid: Xbal

Engineering plasmid name: pAP00050

Strain name: AS_122

### Example 13

### Synthesis of Compound 18

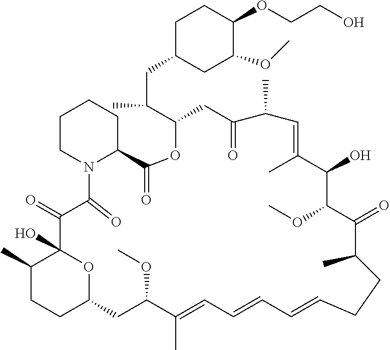 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)CC/C=C/C=C/C=C/1C]

An oven dried 4 mL vial containing a PFTE coated magnetic stir bar was charged with 1.2 mL of toluene and cooled to 0° C. 2-[(tert-Butyldimethylsilyl)oxy]ethanol (381 uL, 344 mg, 1.95 mmol, 22 equiv) and N,N-diisopropylethylamine (768 uL, 568 mg, 4.4 mmol, 50 equiv) were added via pipette. Trifluoromethanesulfonic anhydride (296 uL, 497 mg, 1.76 mmol, 20 equiv) was added dropwise via a syringe, causing the solution to darken and a precipitate to form. The mixture was stirred vigorously at 0° C. for 10 min, and the soluble portion of the mixture was transferred to second 4 mL vial containing (3S,6R,7E,9R,10R,12R,15E,17E,19E,21S,23S,26R,27R,34aS)-9,27-dihydroxy-3 -((R) ((1S,3R,4R)-4-hydroxy-3-methoxycyclohexyl)propan-2-yl)-10,21-dimethoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (80 mg, 0.0088 mmol, 1 equiv). The mixture was stirred at room temperature for 10 min then warmed to 55° C. and monitored by the same LC-MS method described in Example 1. The reaction was nearly complete at 3 h, and was quenched after 3.5 h with an equal volume of water. The mixture was extracted with ethyl acetate three timesand the organic layers was combined, filtered through sodium sulfate, and concentrated under reduced pressure. The crude product was purified via preparative HPLC using the same system in Example 2 by gradient elution of solvent A (5% methanol in water) and solvent B (methanol) at a flow rate of 25 mL/min as the following program: T=0, 60% B; T=1 min, 60% B; T=21 min, 100% B; T=21.1 min, 100% B; T=24 min, 100% B; T=24.1 min, 60% B; T=27 min, 60% B. The fractions were subjected to LC-MS analysis using the same LC-MS method described in Example 1. Fractions containing (3S,6R,7E,9R,10R,12R,15E,17E,19E,21S,23S,26R,27R,34aS)-3-((R)-14(1S,3R,4R)-4-(2-((tert- butyldimethylsilyl)oxy)ethoxy)-3-methoxycyclohexyl)propan-2-yl)-9,27-dihydroxy-10,21-dimethoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (TBS-Compound 18) with pseudo-molecular ion [M+HCOO]⁻ at m/z 1072.6 were combined and concentrated under reduced pressure to yield TBS-Compound 18 (65 mg, 69% yield).

TBS-Compound 18 (40 mg, 0.113 mmol, 1 equiv) was dissolved in 1.1 mL methanol in a 4 mL glass vial at room temperature, followed by aqueous HCl (1 M, 113 μL, 0.113 mmol, 1 equiv). The solution was stirred at room temperature for 30 min, then evaporated under a stream of nitrogen. The residue was dissolved ethyl acetate (2 mL) and washed with an equal volume of water. The organic phase was filtered through sodium sulfate and concentrated under reduced pressure. The crude product was purified by preparative HPLC purification using the same procedure described as “Prep Step A” in Example 2. Fractions containing (3S,6R,7E,9R,10R,12R,15E,17E,19E,21S,23S,26R,27R,34aS)-9,27-dihydroxy-34(R)-1-((1S,3R,4R)-4-(2-hydroxyethoxy)-3-methoxycyclohexyl)propan-2-yl)-10,21-dimethoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Compound 18) with pseudo-molecular ion [M+HCOO]⁻ at m/z 988.6. These fractions were combined and concentrated under reduced pressure to afford Compound 18 (13 mg, 39% yield).

Compound 18: HPLC retention time: 1.025 min; LC-MS m/z: [M+HCOO]⁻ calc. 988.6 for C₅₃H₈₂NO₁₆, found 988.5. LC-MS/MS m/z: Frag. A calc. 365.2 for C₂₁H₃₃O₅, found 365.3; Frag. B calc. 576.3 for C₃₁H₄₆NO₉, found 576.4.

### Example 14

### Synthesis of Compound 19

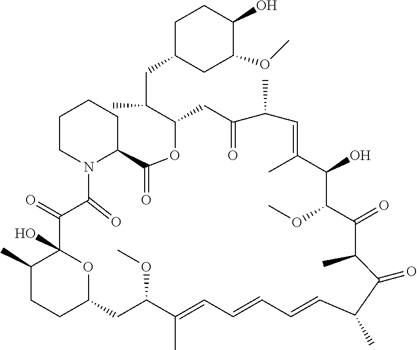 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C(=O)[C@H](C)/C=C/C=C/C=C/1C]

The synthesis outlined in Example 1 was conducted to generate the strain to produce (3S,6R,7E,9R,10R,12R,14R,15E,17E,19E,21S,23S,26R,27R,34aS)-9,27-dihydroxy-3-((R)-1-((1S,3R,4R)-4-hydroxy-3-methoxycyclohexyl)propan-2-yl)-10,21-dimethoxy-6,8,12,14,20,26-hexamethyl-9,10,14,21,22,23,24,25,26,27,32,33,34,34a-tetradecahydro-3H-23,27-epoxypyrido[2,1-c] [1] oxa[4]azacyclohentriacontine-1,5,11,13,28,29(4H,6H,12H,31H)-hexaone (Compound 19) except three DNA fragments, including Left Arm, Right Arm, and pUC₉ vector, were used to assemble the intermediate plasmid.

TABLE 11
Templates and primers used for Compound 19:

| Target | | Fragment | | |
|---|---|---|---|---|
| fragment | Template | Length | Primers | |
| Left Arm | S. rapamycinicus | 2 kb | KR7_L_F | GCTCGGTACCCGGGGATCCTTCT |
| | genomic DNA | | | AGATCGCCGACGACAGCGGCCGA |
| | | | KR7_L_R | AACGCGTTGGCCGCCGCGAAGTT |
| | | | | GCCCTGTCCCCCGCTGC |
| Right Arm | S. rapamycinicus | 1.9 kb | KR7_R_F | GCGGGGGACAGGGCAACTTCGCG |
| | genomic DNA | | | GCGGCCAACGCGTTCCT |
| | | | KR7_R_R | TGCATGCCTGCAGGTCGACTTCT |
| | | | | AGAACTGATCCCGAAGGACGACA |
| | | | | CGCC |
| pUC19 | pUC19 | 2 kb | pUC19_XbaI_L_F | TCTAGAAGTCGACCTGCAGGCAT |
| vector | | | | GCA |
| | | | pUC19_XbaI_L_R | TCTAGAAGGATCCCCGGGTACCG |
| | | | | AGC |

Intermediate plasmid name: pAP00056

Restriction enzyme to construct the engineering plasmid: EcoRI-HindIII

Engineering plasmid name: pAP00068

Strain name: AS_130

### Example 15

### Synthesis of Compound 20

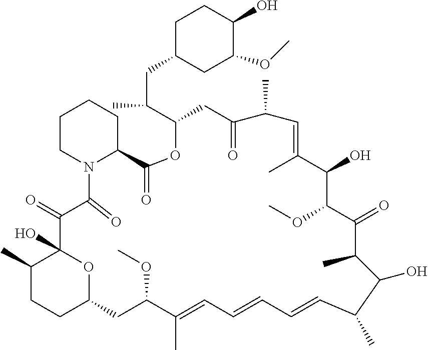 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C(O)[C@H](C)/C=C/C=C/C=C/1C]

The synthesis outlined in Example 1 was conducted to generate the strain to produce (3S,6R,7E,9R,10R,12R,14R,15E,17E,19E,21S,23S,26R,27R,34aS)-9,13,27-trihydroxy-3 -((R)-1-((1S,3R,4R)-4-hydroxy-3-methoxycyclohexyl)propan-2-yl)-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c] [1] oxa[4]azacyclohentriacontine-1,5,11,28,29(4H,6H,31H)-pentaone (Compound 20) except three DNA fragments, including Left Arm, Right Arm, and pUC₉ vector, were used to assemble the intermediate plasmid.

TABLE 12
Templates and primers used for Compound 20:

| Target | | Fragment | | |
|---|---|---|---|---|
| fragment | Template | Length | Primers | |
| Left Arm | S. rapamycinicus | 2 kb | DH7_L_F | GCTCGGTACCCGGGGATCCTTCTAGAT |
| | genomic DNA | | | CGCGGACAGTGCGGATGGTGC |
| | | | DH7_L_R | ACACTGCCCCGCACCGCGAAATCAGCC |
| | | | | AGCCAGGCATGCGTGG |
| Right Arm | S. rapamycinicus | 1.8 kb | DH7_R_F | ATGCCTGGCTGGCTGATTTCGCGGTGC |
| | genomic DNA | | | GGGGCAGTGTGCT |
| | | | DH7_R_R | TGCATGCCTGCAGGTCGACTTCTAGAA |
| | | | | TTTCCCGGAAGCCAGTGGTACGCGA |
| pUC19 | pUC19 | 2 kb | pUC19_XbaI_L_F | TCTAGAAGTCGACCTGCAGGCATGCA |
| vector | | | pUC19_XbaI_L_R | TCTAGAAGGATCCCCGGGTACCGAGC |

Intermediate plasmid name: pAP00062

Restriction enzyme to construct the engineering plasmid: XbaI-Eco53KI for pAP00062, XbaI-EcoRV for pKC₁₁₃₉

Engineering plasmid name: pAP00074

Strain name: AS_142

While preferred embodiments of the present disclosure have been shown and described herein, it may be obvious to those skilled in the art that such embodiments are provided by way of example only. Numerous variations, changes, and substitutions may now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A compound, or a pharmaceutically-acceptable salt or solvate thereof, having a structure represented by a structure of Formula (I): 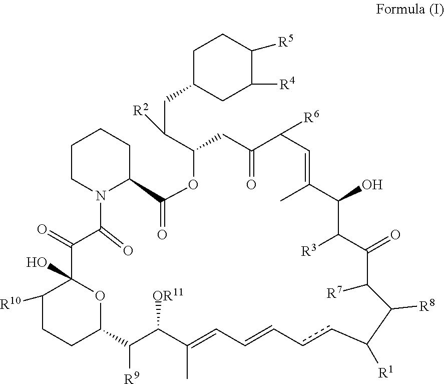
wherein, [symbol]
is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC (═O)NR²³R²⁴, —NR²¹(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O) OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)²R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═) NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R₂₂, —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R_{1c}, and R^{1d} is independently oxo, halogen, —CN, —ORa, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of Formula (I) to a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
(c) when R¹ is H or methyl, at least one of (i) R⁶ is not CH₃; (ii) R⁷ is not —CH₃; (iii) R⁸ is not hydrogen; (iv) R⁹ is not hydrogen; (v) R¹⁰ is not —CH₃, (vi) R⁴ is not —OCH₃; or (vii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

2. The compound of claim 1, wherein R¹ is hydrogen.

3. The compound of claim 1, wherein R¹ is CH₃.

4. The compound of claim 1, wherein R² is CH₃.

5. The compound of claim 1, wherein R³ is OCH₃.

6. The compound of claim 1, wherein R⁴ is OCH₃.

7. The compound of claim 1, wherein R⁴ is OH.

8. The compound of claim 1, wherein R⁵ is selected from the group consisting of:

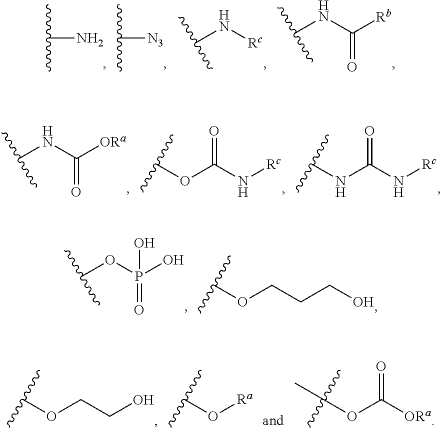

9. The compound of claim 1, wherein R⁶ is CH₃.

10. The compound of claim 1, wherein R⁷ is CH₃.

11. The compound of claim 1, wherein R⁸ is hydrogen.

12. The compound of claim 1, wherein R⁹ is hydrogen.

13. The compound of claim 1, wherein R¹⁰ is CH₃.

14. The compound of claim 1, wherein R¹¹ is CH₃.

15. The compound of claim 1, wherein R¹ is an optionally substituted alkyl.

16. The compound of claim 1, wherein R² is an optionally substituted alkyl.

17. The compound of claim 1, wherein R³ is an optionally substituted alkoxy.

18. The compound of claim 1, wherein R⁴ is OH or an optionally substituted alkoxy.

19. The compound of claim 1, wherein at least one of R¹, R⁶, R⁷, R⁸, R⁹ or R¹⁰ is hydrogen, C₁-C₈ alkyl or C₂-C₈ alkenyl, each of which is unsubstituted or substituted with one or more R^{1e}, wherein R^{1e} is independently halogen, hydroxy, oxo, or phenyl.

20. The compound of claim 1, wherein at least one of R¹, R⁶, R⁷, R⁸, R⁹ or R¹⁰ is hydrogen or a group selected from the group consisting of:

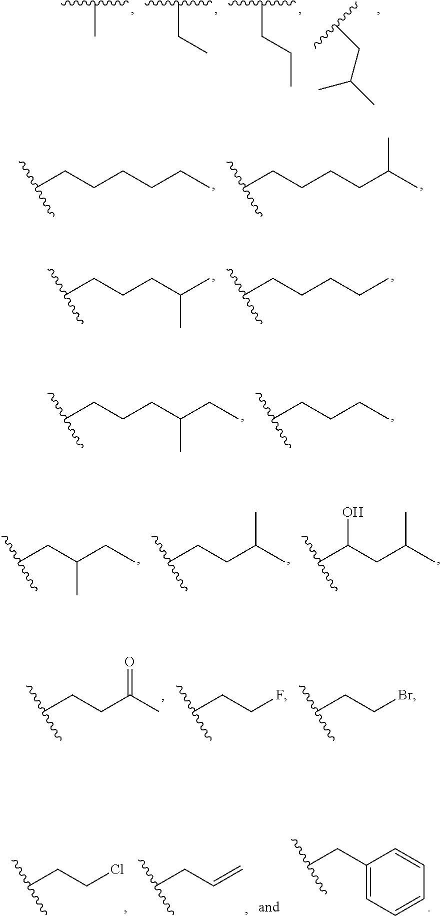

21. The compound of claim 1, wherein the compound is selected from group from the consisting of:

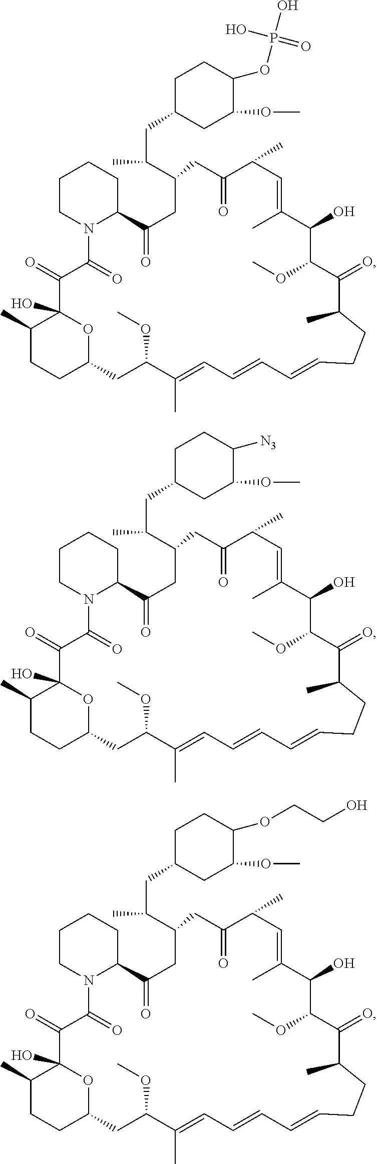

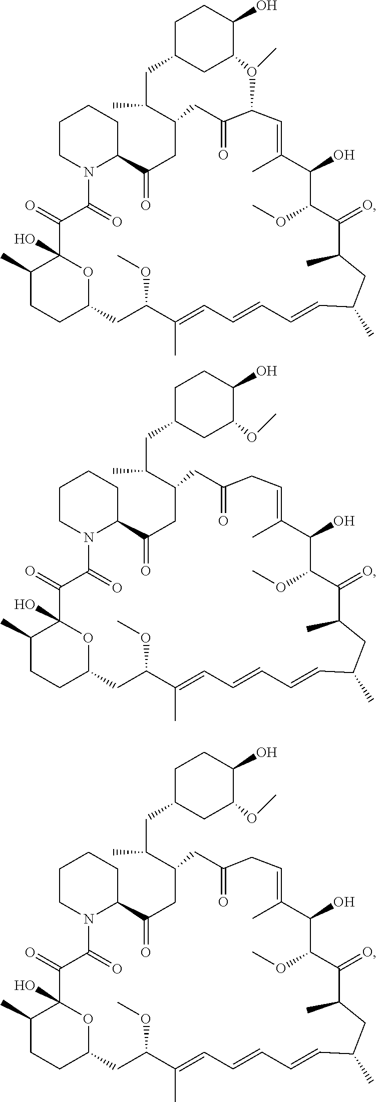

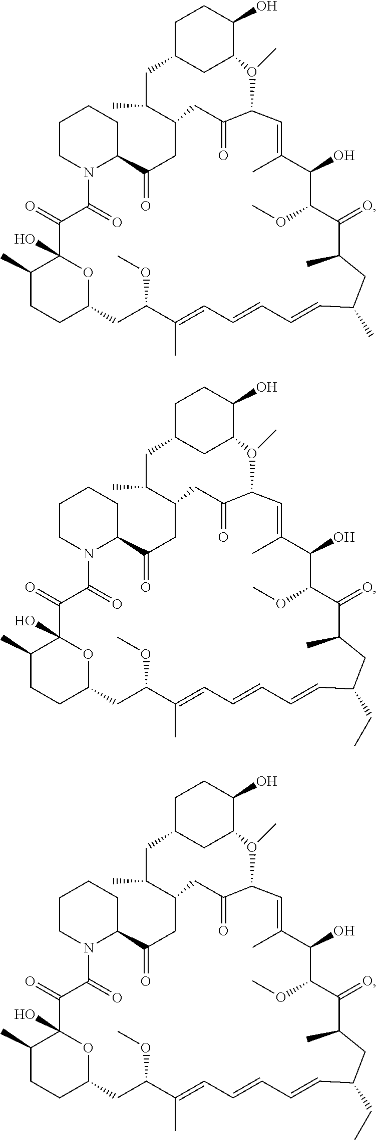

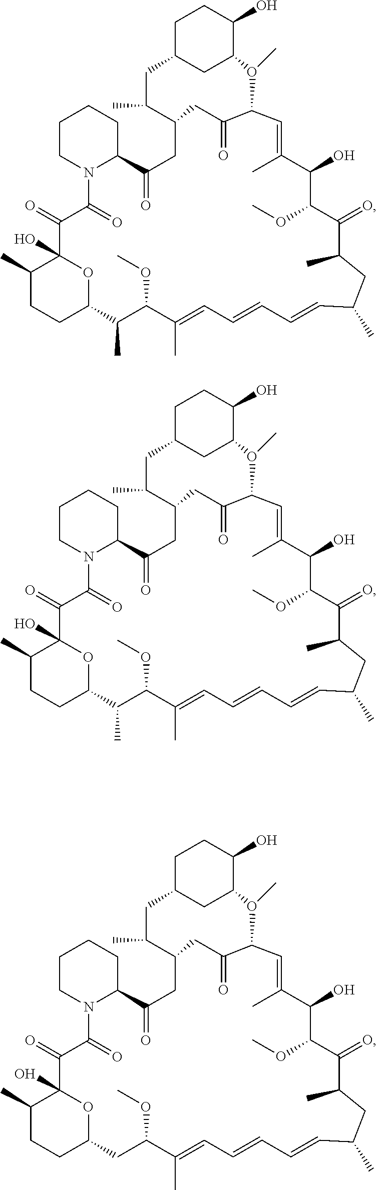

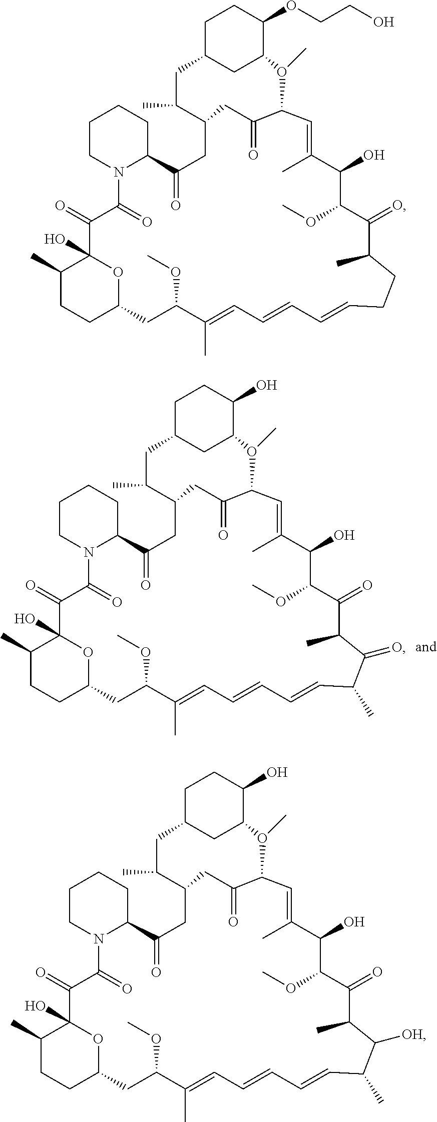

22. The compound of claim 1, or a pharmaceutically-acceptable salt or solvate thereof, having a structure represented by a structure of Formula (I-A): 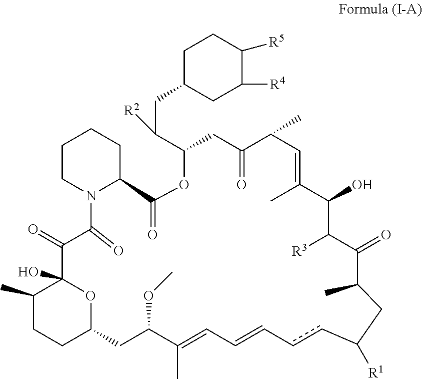
wherein, [symbol]
is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, NR²¹C(═O)R²², —NR²¹C(═O)OR²¹, NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —O(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH, or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, -—C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
or a conjugate of the compound of Formula (I-A) to a biomolecule;
wherein
(a) when R¹ is hydrogen, at least one of (i) R² is not CH₃, (ii) R³ is not OCH₃, or (iii) R⁴ is not —OCH₃;
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂; and
(c) when R¹ is H or methyl, at least one of (i) R⁴ is not —OCH₃; or (ii) R⁵ is not —OH or —OC(═O)R^{b}, wherein R^{b} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted.

23. The compound of claim 1, or a pharmaceutically-acceptable salt or solvate thereof, having a structure represented by a structure of Formula (I-B): 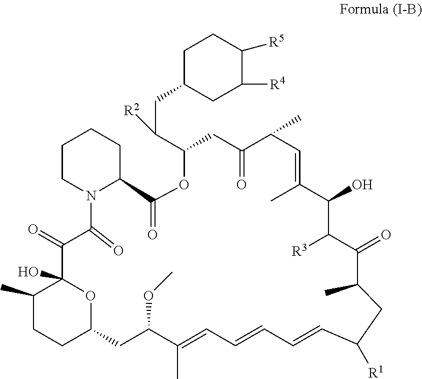
wherein,
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a1}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
R^{a1} is C₁-C₈ alkyl, C₂-C₈ alkenyl, cycloalkyl, heterocycloalkyl, aryl, or heteroaryl; wherein the C₁-C₈ alkyl, cycloalkyl, heterocycloalkyl, aryl, and heteroaryl are each independently optionally substituted with one or more R²⁰;
or a conjugate of the compound of Formula (I-B) to a biomolecule;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not —O(CH₂)₂OH —O(CH₂)₂OCH₂CH₃; —OC(═O)C(CH₂OH)₂CH₃; or OP(═O)(CH₃)₂.

24. A conjugate of a compound of Formula (I), or a pharmaceutically-acceptable salt thereof, to a biomolecule 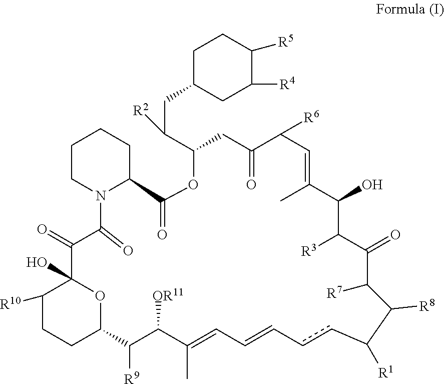
wherein, [symbol]
is a single or a double bond;
R¹ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR₂₃R₂₄, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹, C₁-C₈ alkyl, C₂-C₈ alkenyl, cycloalkyl, heterocycloalkyl, aryl, or heteroaryl;
wherein the C₁-C₈ alkyl, C₂-C₈ alkenyl, cycloalkyl, heterocycloalkyl, aryl, and heteroaryl are each independently optionally substituted with one or more R²⁰;
R⁴ is —OH or —OCH₃;
R⁵ is —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)_{R}²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
R¹¹ is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

25. A conjugate, or a pharmaceutically-acceptable salt or solvate thereof, having a structure represented by a structure of Formula (I-C): 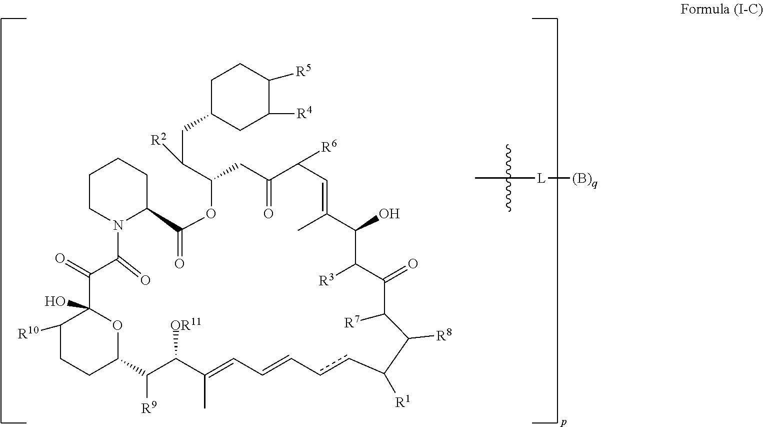
wherein, [symbol]
is a single or a double bond;
each R¹ is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, 13 NR²¹C(═O)NR²³R²⁴, —NR²¹S (═O)₂NR²³R²⁴, —NR²¹C(═O)R²², —NR²¹(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl;
wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}—NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)₂R²², —S(═O)²NR²³R²⁴, C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)_{R}²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
B is a biomolecule;
p is 1-100; and
q is 1-100;
wherein
(a) when R¹ is hydrogen, at least one of (i) R² is not CH₃, (ii) R³ is not OCH₃, or (iii) R⁴ is not —OCH₃; and
(b) when R¹ is CH₃, at least one of (i) R² is not CH₃, (ii) R³ is not hydrogen, —CH₃ or —OCH₃, (iii) R⁴ is not —OCH₃; or (iv) R⁵ not OH, —O(CH₂)₂OH —O(CH₂)₂OCH₂CH₃; —OC(═O)C(CH₂OH)₂CH₃; or OP(═O)(CH₃)₂.

26. An antibody-drug conjugate (ADC) having a structure represented by a structure of Formula (I-D): 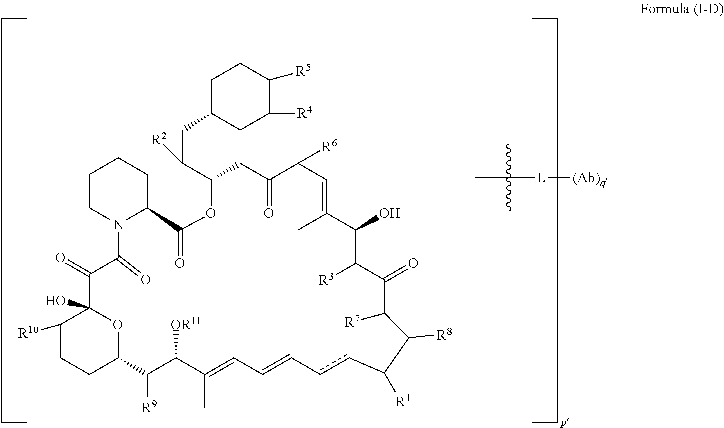
wherein, [symbol]
is a single or a double bond;
each R¹ is independently hydrogen, halogen, —CN, —OR²¹, —SR²¹, —S(═O)_{R}²², —S(═O)₂R²², —NO₂, —NR²³R²⁴, —NR²¹S(═O)₂R²², S(═O)₂NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹(═O)NR²³R²⁴, —NR²¹S (═O)₂NR²³R²⁴, —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R² is independently hydrogen, halogen, —CN, —OR²¹, —SR²¹, —S(═O)R²², —S(═O)₂R²², —NO₂, —NR²³R²⁴, —NR²¹S(═O)₂R²², S(═O)₂NR²³R²⁴, —C(═O)R²², —C₁-C₈—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R³ is independently hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C₁-C₈—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R⁴ is independently —OH or —OCH₃;
each R⁵ is independently —OR^{a}, —OC(═O)R^{b}, —OC(═O)OR^{a}, —OC(═O)—NR^{c}R^{d}, —NR^{c}R^{d}, —NR^{c}—C(═O)R^{b}, —NR^{c}—C(═O)OR^{a}, —NR^{c}—C(═O)—NR^{c}R^{d}, —OP(═O)(OR^{a})₂, —OP(═O)(R^{b})₂ or N₃;
R⁶, R⁷, R⁸, R⁹ and R¹⁰ are each independently hydrogen, halogen, —CN, —OR²¹, —SR²¹, —S(═O)R²², —S(═O)₂R²², —NO₂, —NR²³R²⁴, —NR²¹S(═O)²R²², —S(═O)²NR²³R²⁴, —C(═O)R²², —OC(═O)R²², —C₁-C₈-alkyl-C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R¹¹ independently is hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R²⁰;
each R²⁰ is independently halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, Cₗ-C₈ alkyl, C₂-C₈ alkenyl, C₁-C₈ haloalkyl, C₁-C₈ hydroxyalkyl, or phenyl;
each R²¹ is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one R^{1a};
each R²² is independently hydrogen, —CN, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1b};
R²³ and R²⁴ are each independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} is independently oxo, halogen, —CN, —OR^{a}, —SR^{b}, —S(═O)₂R^{b}, —NR^{c}R^{d}, —S(═O)₂NR^{c}R^{d}, —C(═O)R^{b}, —OC(═O)R^{b}, —C(═O)OR^{a}, —C(═O)SR^{b}, —OC(═O)OR^{a}, —OC(═O)SR^{b}, —OC(═O)SR^{b}, —C(═O)NR^{c}R^{d}, —OC(═O)NR^{c}R^{d}, —NR^{a}C(═O)NR^{c}R^{d}, —NR^{a}C(═O)R^{b}, C₁-C₈ alkyl, —C₂-C₈ alkenyl, C₁-C₈ haloalkyl, C₁-C₈ hydroxyalkyl, or phenyl;
each R^{a}, R^{b}, R^{c}, and R^{d} is independently hydrogen, C₁-C₈ alkyl, C₂-C₈ alkenyl, cycloalkyl, heterocycloalkyl, aryl, or heteroaryl; wherein the C₁-C₈ alkyl, C₂-C₈ alkenyl, cycloalkyl, heterocycloalkyl, aryl, and heteroaryl are each independently optionally substituted;
or R^{c} and R^{d} are taken together with the nitrogen atom to which they are attached to form a heterocycloalkyl which is optionally substituted;
L is a linker;
Ab is an antibody or an antibody fragment;
p′ is 1-100; and
q′ is 1-100.

27. A pharmaceutical composition comprising a compound of claim 1, and at least one pharmaceutically-acceptable excipient.

28. The pharmaceutical composition of claim 27, wherein the pharmaceutical composition is in a unit dosage form.

29. The pharmaceutical composition of claim 27, in a form suitable for oral, parenteral, rectal, ocular, intravenous or otic administration, or administration by inhalation.

30. A kit comprising a pharmaceutical composition of claim 27.
